(19) United States
(12) Patent Application Publication
(10) Pub. No.: US 2010/0074934 A1
(43) Pub. Date: Mar. 25, 2010

(54) MEDICAL IMPLANTS WITH A COMBINATION OF COMPOUNDS

(76) Inventor: William L. Hunter, Vancouver (CA)

Correspondence Address: KNOBBE MARTENS OLSON & BEAR LLP, 2040 MAIN STREET, FOURTEENTH FLOOR, IRVINE, CA, 92614, US

(21) Appl. No.: 12/518,962

(22) PCT Filed: Dec. 12, 2007
(86) PCT No.: PCT/CA07/02267

§ 371 Date: Nov. 3, 2009

Related U.S. Application Data
(60) Provisional application No. 60/869,905, filed on Dec. 13, 2006.

(51) Int. Cl. A61K 9/00; A61K 31/519; A61P 9/00

(52) U.S. Cl. 424/422; 514/262.1

(57) ABSTRACT

Implants are associated with a combination of paclitaxel or derivatives and dipyridamole or derivatives in order to inhibit fibrosis that may otherwise occur when the implant is placed within an animal. Exemplary implants include intravascular implants (e.g., coronary and peripheral vascular stents, catheters, balloons), non-vascular stents, pumps and sensors, vascular grafts, perivascular devices, implants for hemodialysis access, vena cava filters, implants for providing an anastomotic connection, electrical devices, intraocular implants, and soft tissue implants and fillers.

### RELATED APPLICATIONS

This application claims priority to U.S. Provisional Application Ser. No. 60/869,905, filed Dec. 13, 2006, which, where permitted, is incorporated by reference herein in its entirety

### BACKGROUND

1. Field of this Disclosure

The present disclosure relates generally to pharmaceutical compositions, medical devices, combinations thereof, and methods for making and using same.

2. Description of the Related Art

The clinical function of numerous medical implants and devices is dependent upon the device being able to effectively maintain an anatomical, or surgically created, space or passageway. Unfortunately, many devices implanted in the body are subject to a “foreign body” response from the surrounding host tissues. In particular, injury to tubular anatomical structures (such as blood vessels, the gastrointestinal tract, the male and female reproductive tract, the urinary tract, sinuses, spinal nerve root canals, lacrimal ducts, Eustachian tubes, the auditory canal, and the respiratory tract) from surgery and/or injury created by the implantation of medical devices can lead to a well known clinical problem called “stenosis” (or narrowing). Stenosis occurs in response to irritation, trauma, or injury to the epithelial lining or the wall of the body tube during an interventional procedure, including virtually any manipulation which attempts to relieve obstruction of the passageway, and is a major factor limiting the effectiveness of invasive treatments for a variety of diseases to be described later.

Stenosis (or “restenosis” if the problem recurs after an initially successful attempt to open a blocked passageway) is a form of response to injury leading to wall thickening, narrowing of the lumen, and loss of function in the tissue supplied by the particular passageway. Physical injury during an interventional procedure results in damage to epithelial lining of the tube and the underlying connective tissue cells (typically smooth muscle cells or SMCs) that make up the wall. The damaged cells, particularly SMCs, release cytokines, which recruit inflammatory cells such as macrophages, lymphocytes and neutrophils (i.e. types of white blood cells) into the area. The white blood cells in turn release a variety of additional cytokines, growth factors, and tissue degrading enzymes that influence the behavior of the constituent cells of the wall (primarily epithelial cells and SMCs). Stimulation of the SMCs induces them to migrate into the inner aspect of the body passageway (often called the “intima”), proliferate and secrete an extracellar matrix—effectively filling all or parts of the lumen with reactive, fibrous scar tissue. Collectively, this creates a thickening of the intimal layer (known in some tissues as “neointimal hyperplasia”) that narrows the lumen of the passageway and can be significant enough to obstruct its lumen. Although this reaction leading to narrowing or obstruction of the body passageway is most often described for vascular obstruction following a therapeutic manipulation, it should be noted that excessive scar tissue growth that creates an unwanted a space-occupying lesion can occur following almost any surgical intervention that traumatizes native tissue.

### BRIEF SUMMARY OF THIS DISCLOSURE

In one aspect, the present disclosure provides a combination comprising paclitaxel and dipyridamole. In one aspect, the combination inhibits one or more processes in the production of excessive fibrous (scar) tissue. Furthermore, compositions and methods are described for associating medical devices and implants with a composition such that paclitaxel and dipyridamole are delivered in therapeutic levels over a period sufficient to allow normal healing to occur. In addition, numerous specific implants and devices are described that produce superior clinical results as a result of being associated with a combination of paclitaxel and dipyridamole that reduce excessive scarring and fibrous tissue accumulation as well as other related clinical advantages.

In one aspect, non-toxic compositions are provided that comprise paclitaxel and dipyridamole, wherein the paclitaxel has a biological effect, and the effect is greater in the presence of dipyridamole than in the absence of dipyridamole.

In another aspect, compositions are provided that comprise a combination of paclitaxel and dipyridamole, wherein the biological effect of the combination is greater than the sum of the effects of dipyridamole or paclitaxel acting alone.

In yet another aspect, compositions are provided that include a combination of paclitaxel and dipyridamole, wherein the weight ratio of dipyridamole to paclitaxel exceeds 0.06 to 1.0.

In yet another aspect, medical devices are provided that include a composition in which paclitaxel is present in an amount ranging from about 0.01 to about 1.0 μg/mm² and dipyridamole is present in an amount ranging from about 0.05 to about 50 μg/mm² of device surface area.

In yet another aspect, medical devices are provided that include a composition in which paclitaxel is present in an amount ranging from about 0.1 to about 0.6 μg/mm² and dipyridamole is present in an amount ranging from about 0.5 to about 5 μg/mm² of device surface area.

In yet other aspects, medical devices and implants are provided which comprise a combination of paclitaxel and dipyridamole or a composition that comprises a combination of paclitaxel and dipyridamole. Implants may be associated with a combination of compounds (e.g., paclitaxel and dipyridamole) in order to inhibit fibrosis that may otherwise occur when the implant is placed within an animal. Exemplary implants include intravascular implants (e.g., coronary and peripheral vascular stents, catheters, balloons), pumps (e.g., drug delivery pumps) and sensors, non-vascular stents, vascular grafts, perivascular devices, implant for hemodialysis access, implants for providing an anastomotic connection, electrical devices, intraocular implants, and soft tissue implants and fillers.

In other aspects, methods of making and using the compositions, medical devices and implants of this disclosure are described.

These and other aspects of the present disclosure will become evident upon reference to the following detailed description and attached drawings. In addition, various references are set forth herein which describe in more detail certain procedures and/or compositions (e.g., polymers), and are therefore incorporated by reference in their entirety.

In one aspect, a device provided that comprises a medical device, paclitaxel and dipyridamole, wherein paclitaxel is present in an amount ranging from about 0.01 to about 1.0 μg/mm² and dipyridamole is present in an amount ranging from about 0.05 to about 50 μg/mm² of medical device surface area. In some aspects, paclitaxel is present in an amount ranging from about 0.1 to about 0.6 μg/mm² and dipyridamole is present in an amount ranging from about 0.5 to about 5 μg/mm² of medical device surface area.

In another aspect, a device is provided that comprises a medical device, paclitaxel and dipyridamole, wherein paclitaxel is present in an amount ranging from about 0.01 to about 1.0 μg/mm² and dipyridamole is present in an amount ranging from about 0.01 to about 1.0 μg/mm² of medical device surface area.

In some aspects, the device further comprises a polymer. In some such aspects, the polymer is a non-biodegradable polymer. In some such aspects, the polymer is a biodegradable polymer.

In some aspects, the medical device is an intravascular device selected from a catheter, a balloon, and a vena cava filter.

In some aspects, the medical device is selected from drug delivery pumps, sensors, non-vascular stents, vascular grafts, perivascular devices, implants for hemodialysis access, implants for providing an anastomotic connection, electrical devices, intraocular implants, and soft tissue implants and tissue fillers.

In some aspects, the medical device is a coronary stent or a peripheral vascular stent.

In some aspects of the device, the paclitaxel has a biological effect, and the effect is greater in the presence of dipyridamole than in the absence of dipyridamole, and the biological effect is to minimize formation of neointimal hyperplasia.

In another aspect, a composition is provided comprising paclitaxel and dipyridamole, wherein the weight ratio of dipyridamole to paclitaxel exceeds 0.06 to 1.0. In some aspects, the paclitaxel has a biological effect, and the biological effect is greater in the presence of dipyridamole than in the absence of dipyridamole. In some aspects, the composition comprises a combination of paclitaxel and dipyridamole, wherein the biological effect of the combination is greater than the sum of the effects of dipyridamole or paclitaxel acting alone. In some aspects, the composition further comprises a polymer. In some such aspects, the polymer is a non-biodegradable polymer. In some such aspects, the polymer is a biodegradable polymer.

### BRIEF DESCRIPTION OF THE DRAWINGS

FIG. 1 is a bar graph showing the effect of paclitaxel and dipyridamole in the CAM assay.

FIG. 2 is a bar graph showing the effect of paclitaxel (3, 10, 30 μg), dipyridamole (50 μg) and dipyridamole/paclitaxel (50/3 μg, and 50/10 μg) on intimal area after balloon injury in the rat carotid artery.

FIG. 3 is a bar graph showing the effect of paclitaxel (3 μg) and dipyridamole/paclitaxel (50/3 μg, 100/3 μg, 150/3 μg) on intimal area after balloon injury in the rat carotid artery.

FIG. 4 is a bar graph showing the effect of paclitaxel (10 μg) and dipyridamole/paclitaxel (50/10 μg, 100/10 μg, 150/10 μg) on intimal area after balloon injury in the rat carotid artery.

### DETAILED DESCRIPTION OF THIS DISCLOSURE

### Definitions

Prior to setting forth this disclosure, it may be helpful to an understanding thereof to first set forth definitions of certain terms that are used herein.

Any concentration ranges, percentage range, or ratio range recited herein are to be understood to include concentrations, percentages or ratios of any integer within that range and fractions thereof, such as one tenth and one hundredth of an integer, unless otherwise indicated. Also, any number range recited herein relating to any physical feature, such as polymer subunits, size or thickness, are to be understood to include any integer within the recited range, unless otherwise indicated. It should be understood that the terms “a” and “an” as used above and elsewhere herein refer to “one or more” of the enumerated components. For example, “a” polymer refers to one polymer or a mixture comprising two or more polymers. As used herein, the term “about” means±15%.

“Fibrosis,” “Scarring,” or “Fibrotic Response” refers to the formation of fibrous tissue in response to injury or medical intervention. Compounds are provided which inhibit fibrosis or scarring are referred to herein as “fibrosis-inhibiting agents”, “anti-scarring agents,” and the like, where these agents inhibit fibrosis through one or more mechanisms including: inhibiting angiogenesis, inhibiting migration or proliferation of connective tissue cells (such as fibroblasts, smooth muscle cells, vascular smooth muscle cells), reducing ECM production, and/or inhibiting tissue remodeling.

“Association” refers to a state wherein two items are physically connected together, so that to transport one item would necessarily transport some or all of the second item. For example, a stent may be associated with a composition, so that inserting the stent into a patient will necessarily insert into that patient some or all of a composition that has been associated with the stent. “Host”, “Person”, “Subject”, “Patient” and the like are used synonymously to refer to the living being into which a device of the present disclosure is implanted.

“Implanted” refers to having completely or partially placed a device within a host. A device is partially implanted when some of the device reaches, or extends to the outside of, a host.

“Inhibit fibrosis”, “reduce fibrosis” and the like are used synonymously to refer to the action of agents or compositions which result in a statistically significant decrease in the formation of fibrous tissue that can be expected to occur in the absence of the agent or composition.

“Inhibitor” refers to an agent which prevents a biological process from occurring or slows the rate or degree of occurrence of a biological process. The process may be a general one such as scarring or refer to a specific biological action such as, for example, a molecular process resulting in release of a cytokine.

“Analogue” refers to a chemical compound that is structurally similar to a parent compound but differs slightly in composition (e.g., one atom or functional group is different, added, or removed). An analogue may or may not have different chemical or physical properties than the original compound and may or may not have improved biological and/or chemical activity. For example, the analogue may be more hydrophilic, or it may have altered reactivity as compared to the parent compound. The analogue may mimic the chemical and/or biological activity of the parent compound (i.e., it may have similar or identical activity), or, in some cases, may have increased or decreased activity. The analogue may be a naturally or non-naturally occurring (e.g., recombinant) variant of the original compound. An example of an analogue is a mutein (i.e., a protein analogue in which at least one amino acid is deleted, added, or substituted with another amino acid). Other types of analogues include isomers (enantiomers, diasteromers, and the like) and other types of chiral variants of a compound, as well as structural isomers. The analogue may be a branched or cyclic variant of a linear compound. For example, a linear compound may have an analogue that is branched or otherwise substituted to impart certain desirable properties (e.g., improve hydrophilicity or bioavailability).

“Derivative” refers to a chemically or biologically modified version of a chemical compound that is structurally similar to a parent compound and (actually or theoretically) derivable from that parent compound. A “derivative” differs from an “analogue” in that a parent compound may be the starting material to generate a “derivative,” whereas the parent compound may not necessarily be used as the starting material to generate an “analogue.” An analogue may have different chemical or physical properties of the parent compound. For example, the derivative may be more hydrophilic or it may have altered reactivity as compared to the parent compound. Derivatization (i.e., modification) may involve substitution of one or more moieties within the molecule (e.g., a change in functional group). For example, a hydrogen may be substituted with a halogen, such as fluorine or chlorine, or a hydroxyl group (—OH) may be replaced with a carboxylic acid moiety (—COOH). The term “derivative” also includes conjugates, and prodrugs of a parent compound (i.e., chemically modified derivatives which can be converted into the original compound under physiological conditions). For example, the prodrug may be an inactive form of an active agent. Under physiological conditions, the prodrug may be converted into the active form of the compound. Prodrugs may be formed, for example, by replacing one or two hydrogen atoms on nitrogen atoms by an acyl group (acyl prodrugs) or a carbamate group (carbamate prodrugs). More detailed information relating to prodrugs is found, for example, in Fleisher et al., Advanced Drug Delivery Reviews 19 (1996) 115; Design of Prodrugs, H. Bundgaard (ed.), Elsevier, 1985; or H. Bundgaard, Drugs of the Future 16 (1991) 443. The term “derivative” is also used to describe all solvates, for example hydrates or adducts (e.g., adducts with alcohols), active metabolites, and salts of the parent compound. The type of salt that may be prepared depends on the nature of the moieties within the compound. For example, acidic groups, for example carboxylic acid groups, can form, for example, alkali metal salts or alkaline earth metal salts (e.g., sodium salts, potassium salts, magnesium salts and calcium salts, and also salts with physiologically tolerable quaternary ammonium ions and acid addition salts with ammonia and physiologically tolerable organic amines such as, for example, triethylamine, ethanolamine or tris-(2-hydroxyethyl)amine). Basic groups can form acid addition salts, for example with inorganic acids such as hydrochloric acid, sulfuric acid or phosphoric acid, or with organic carboxylic acids and sulfonic acids such as acetic acid, citric acid, benzoic acid, maleic acid, fumaric acid, tartaric acid, methanesulfonic acid or p-toluenesulfonic acid. Compounds that simultaneously contain a basic group and an acidic group, for example a carboxyl group in addition to basic nitrogen atoms, can be present as zwitterions. Salts can be obtained by customary methods known to those skilled in the art, for example by combining a compound with an inorganic or organic acid or base in a solvent or diluent, or from other salts by cation exchange or anion exchange.

“Medical Device”, “Implant”, “Medical Device or Implant”, “implant/device” and the like are used synonymously to refer to any object that is designed to be placed partially or wholly within a patient's body for one or more therapeutic or prophylactic purposes such as for restoring physiological function, alleviating symptoms associated with disease, delivering therapeutic agents, and/or repairing or replacing or augmenting etc. damaged or diseased organs and tissues. While normally composed of biologically compatible synthetic materials (e.g., medical-grade stainless steel, titanium and other metals; polymers such as polyurethane, silicon, PLA, PLGA and other materials) that are exogenous, some medical devices and implants include materials derived from animals (e.g., “xenografts” such as whole animal organs; animal tissues such as heart valves; naturally occurring or chemically-modified molecules such as collagen, hyaluronic acid, proteins, carbohydrates and others), human donors (e.g., “allografts” such as whole organs; tissues such as bone grafts, skin grafts and others), or from the patients themselves (e.g., “autografts” such as saphenous vein grafts, skin grafts, tendon/ligament/muscle transplants). Representative medical devices of particular utility in the present disclosure include, but are not restricted to, vascular stents, gastrointestinal stents, tracheal/bronchial stents, genital-urinary stents, ENT stents, drug delivery balloons and catheters, hemodialysis access devices, vascular grafts, anastomotic connector devices, surgical sheets (e.g., films or meshes), soft tissue implants (such as breast implants, facial implants, tissue fillers, aesthetic implants and the like), implantable electrodes (cardiac pacemakers, neurostimulation devices), implantable sensors, drug delivery pumps, anti-adhesion barriers, and shunts.

“Release of an agent” refers to a statistically significant presence of the agent, or a subcomponent thereof, which has disassociated from the implant/device.

“Biodegradable” refers to materials for which the degradation process is at least partially mediated by, and/or performed in, a biological system. “Degradation” refers to a chain scission process by which a polymer chain is cleaved into oligomers and monomers.

Chain scission may occur through various mechanisms, including, for example, by chemical reaction (e.g., hydrolysis) or by a thermal or photolytic process. Polymer degradation may be characterized, for example, using gel permeation chromatography (GPC), which monitors the polymer molecular mass changes during erosion and drug release. Biodegradable also refers to materials may be degraded by an erosion process mediated by, and/or performed in, a biological system. “Erosion” refers to a process in which material is lost from the bulk. In the case of a polymeric system, the material may be a monomer, an oligomer, a part of a polymer backbone, or a part of the polymer bulk. Erosion includes (i) surface erosion, in which erosion affects only the surface and not the inner parts of a matrix; and (ii) bulk erosion, in which the entire system is rapidly hydrated and polymer chains are cleaved throughout the matrix. Depending on the type of polymer, erosion generally occurs by one of three basic mechanisms (see, e.g., Heller, J., CRC Critical Review in Therapeutic Drug Carrier Systems (1984), 1(1), 39-90); Siepmann, J. et al., Adv. Drug Del. Rev. (2001), 48, 229-247): (1) water-soluble polymers that have been insolubilized by covalent cross-links and that solubilize as the cross-links or the backbone undergo a hydrolytic cleavage; (2) polymers that are initially water insoluble are solubilized by hydrolysis, ionization, or pronation of a pendant group; and (3) hydrophobic polymers are converted to small water-soluble molecules by backbone cleavage. Techniques for characterizing erosion include thermal analysis (e.g., DSC), X-ray diffraction, scanning electron microscopy (SEM), electron paramagnetic resonance spectroscopy (EPR), NMR imaging, and recording mass loss during an erosion experiment. For microspheres, photon correlation spectroscopy (PCS) and other particles size measurement techniques may be applied to monitor the size evolution of erodible devices versus time.

“Synergy” refers to the interaction of two or more agents to produce a biological effect that is greater than the sum of their individual effects. For example, a synergistic effect may be achieved when the individual agents operate on the same molecular targets or biological pathway, or when the agents operate on different molecular targets or biological pathways to provide a clinically superior result.

As discussed above, the present disclosure provides compositions containing paclitaxel and dipyridamole (and/or analogues or derivatives thereof), methods and devices relating to medical implants, which greatly increase the ability to inhibit the formation of reactive scar tissue on, or around, the surface of the device or implant. Described in more detail below are methods for constructing medical implants, compositions and methods for generating medical implants which inhibit fibrosis, and methods for utilizing such medical implants.

### A. Medical Implants

In one aspect, medical implants of the present disclosure are coated with, or otherwise adapted to release an agent which inhibits the formation of scar tissue. Representative examples of medical implants include: vascular stents, angioplasty balloons, inter- and intravascular drug delivery balloons, vascular catheters, gastrointestinal stents, tracheal/bronchial stents, genital-urinary stents, ENT stents, vascular grafts, hemodialysis access devices, anastomotic connector devices, perivascular drug delivery devices (e.g., surgical sheets, films and meshes), soft tissue implants (such as breast implants, facial implants, tissue fillers, aesthetic implants and the like), implantable electrodes (cardiac pacemakers, neurostimulation devices), implantable sensors, drug delivery pumps, tissue barriers (and other implants designed to reduce surgical adhesions) and shunts.

### B. Compounds

The present disclosure provides compositions and devices that include at least two compounds, where those compounds are paclitaxel and dipyrimadole and/or analogues or derivatives thereof.

Paclitaxel is a highly derivatized diterpenoid (Wani et al., J. Am. Chem. Soc. 93:2325, 1971) which has been obtained from the bark of Taxus brevifolia (Pacific Yew) and Taxomyces Andreanae and Endophytic Fungus of the Pacific Yew (Stierle et al., Science 60:214-216, 1993). Paclitaxel is commercially available in combination with cremephor, as sold by Bristol Myers Squibbk, New York, N.Y., as TAXOL. Paclitaxel is also available from chemical supply houses. In the older literature, paclitaxel may be referred to as taxol or Taxol (see, e.g., The Chemistry of Taxol by David G. I. Kingston, Pharmac. Ther. Vol. 52, pp. 1-34, 1991).

In lieu of paclitaxel, one may utilize a paclitaxel-like compound, such as a paclitaxel analogue, derivative, conjugate, or prodrug thereof. Examples include TAXOTERE (Aventis Pharmaceuticals, France), docetaxel, 10-desacetyl analogues of paclitaxel and 3′N-desbenzoyl-3′N-t-butoxy carbonyl analogues of paclitaxel, may be readily prepared utilizing techniques known to those skilled in the art (see, e.g., Schiff et al., Nature 277:665-667, 1979; Long and Fairchild, Cancer Research 54:4355-4361, 1994; Ringel and Horwitz, J. Nat'l Cancer Inst. 83(4):288-291, 1991; Pazdur et al., Cancer Treat. Rev. 19(4):351-386, 1993; WO 94/07882; WO 94/07881; WO 94/07880; WO 94/07876; WO 93/23555; WO 93/10076; WO94/00156; WO 93/24476; EP 590267; WO 94/20089; U.S. Pat. Nos. 5,294,637; 5,283,253; 5,279,949; 5,274,137; 5,202,448; 5,200,534; 5,229,529; 5,254,580; 5,412,092; 5,395,850; 5,380,751; 5,350,866; 4,857,653; 5,272,171; 5,411,984; 5,248,796; 5,248,796; 5,422,364; 5,300,638; 5,294,637; 5,362,831; 5,440,056; 4,814,470; 5,278,324; 5,352,805; 5,411,984; 5,059,699; 4,942,184; Tetrahedron Letters 35(52):9709-9712, 1994; J. Med. Chem. 35:4230-4237, 1992; J. Med. Chem. 34:992-998, 1991; J. Natural Prod. 57(10):1404-1410, 1994; J. Natural Prod. 57(11):1580-1583, 1994; J. Am. Chem. Soc. 110:6558-6560, 1988), or obtained from a variety of commercial sources, including for example, Sigma Chemical Co., St. Louis, Mo. (T7402 from Taxus brevifolia).

In certain aspects, the paclitaxel-type compound is 7-deoxy-docetaxol, a 7,8-cyclopropataxane, an N-substituted 2-azetidones, a 6,7-epoxy paclitaxel, 6,7-modified paclitaxel such as 6,7-epoxy pactliaxel, 10-desacetoxytaxol, 10-deacetyltaxol (from 10-deacetylbaccatin III), phosphonooxy and carbonate derivatives of taxol, taxol 2′,7-di(sodium 1,2-benzenedicarboxylate, 10-desacetoxy-11,12-dihydrotaxol-10,12(18)-diene derivatives, 10-desacetoxytaxol, Protaxol (2′- and/or 7-O-ester derivatives), (2′- and/or 7-O-carbonate derivatives), asymmetric synthesis of taxol side chain, fluoro taxols, 9-deoxotaxane, (13-acetyl-9-deoxobaccatine III, 9-deoxotaxol, 7-deoxy-9-deoxotaxol, 10-desacetoxy-7-deoxy-9-deoxotaxol, derivatives containing hydrogen or acetyl group and a hydroxy and tert-butoxycarbonylamino, sulfonated 2′-acryloyltaxol and sulfonated 2′-O-acyl acid taxol derivatives, succinyltaxol, 2′-γ-aminobutyryltaxol formate, 2′-acetyl taxol, 7-acetyl taxol, 7-glycine carbamate taxol, 2′-OH-7-PEG(5000) carbamate taxol, 2′-benzoyl and 2′,7-dibenzoyl taxol derivatives, other prodrugs (2′-acetyltaxol; 2′,7-diacetyltaxol; 2′ succinyltaxol; 2′-(beta-alanyl)-taxol); 2′ gamma-aminobutyryltaxol formate; ethylene glycol derivatives of 2′-succinyltaxol; 2′-glutaryltaxol; 2′-(N,N-dimethylglycyl)taxol; 2′-(2-(N,N-dimethylamino)propionyl)taxol; 2′ orthocarboxybenzoyl taxol; 2′ aliphatic carboxylic acid derivatives of taxol, Prodrugs {2′(N,N-diethylaminopropionyl)taxol, 2′(N,N-dimethylglycyl)taxol, 7(N,N-dimethylglycyl)taxol, 2′,7-di-(N,N-dimethylglycyl)taxol, 7(N,N-diethylaminopropionyl)taxol, 2′,7-di(N,N-diethylaminopropionyl)taxol, 2′-(L-glycyl)taxol, 7-(L-glycyl)taxol, 2′,7-di(L-glycyl)taxol, 2′-(L-alanyl)taxol, 7-(L-alanyl)taxol, 2′,7-di(L-alanyl)taxol, 2′-(L-leucyl)taxol, 7-(L-leucyl)taxol, 2′,7-di(L-leucyl)taxol, 2′-(L-isoleucyl)taxol, 7-(L-isoleucyl)taxol, 2′,7-di(L-isoleucyl)taxol, 2′-(L-valyl)taxol, 7-(L-valyl)taxol, 2′7-di(L-valyl)taxol, 2′-(L-phenylalanyl)taxol, 7-(L-phenylalanyl)taxol, 2′,7-di(L-phenylalanyl)taxol, 2′-(L-prolyl)taxol, 7-(L-prolyl)taxol, 2′,7-di(L-prolyl)taxol, 2′-(L-lysyl)taxol, 7-(L-lysyl)taxol, 2′,7-di(L-lysyl)taxol, 2′-(L-glutamyl)taxol, 7-(L-glutamyl)taxol, 2′,7-di(L-glutamyl)taxol, 2′-(L-arginyl)taxol, 7-(L-arginyl)taxol, 2′,7-di(L-arginyl)taxol}, taxol analogues with modified phenylisoserine side chains, TAXOTERE, (N-debenzoyl-N-tert-(butoxycarbonyl)-10-deacetyltaxol, and taxanes (e.g., baccatin III, cephalomannine, 10-deacetylbaccatin III, brevifoliol, yunantaxusin and taxusin); and other taxane analogues and derivatives, including 14-beta-hydroxy-10 deacetylbaccatin III, debenzoyl-2-acyl paclitaxel derivatives, benzoate paclitaxel derivatives, phosphonooxy and carbonate paclitaxel derivatives, sulfonated 2′-acryloyltaxol; sulfonated 2′-O-acyl acid paclitaxel derivatives, 18-site-substituted paclitaxel derivatives, chlorinated paclitaxel analogues, C4 methoxy ether paclitaxel derivatives, sulfonamide taxane derivatives, brominated paclitaxel analogues, Girard taxane derivatives, nitrophenyl paclitaxel, 10-deacetylated substituted paclitaxel derivatives, 14-beta-hydroxy-10 deacetylbaccatin III taxane derivatives, C7 taxane derivatives, C10 taxane derivatives, 2-debenzoyl-2-acyl taxane derivatives, 2-debenzoyl and -2-acyl paclitaxel derivatives, taxane and baccatin III analogues bearing new C2 and C4 functional groups, n-acyl paclitaxel analogues, 10-deacetylbaccatin III and 7-protected-10-deacetylbaccatin III derivatives from 10-deacetyl taxol A, 10-deacetyl taxol B, and 10-deacetyl taxol, benzoate derivatives of taxol, 2-aroyl-4-acyl paclitaxel analogues, ortho-ester paclitaxel analogues, 2-aroyl-4-acyl paclitaxel analogues and 1-deoxy paclitaxel and 1-deoxy paclitaxel analogues.

In one aspect, the paclitaxel-like compound has the formula (C1):

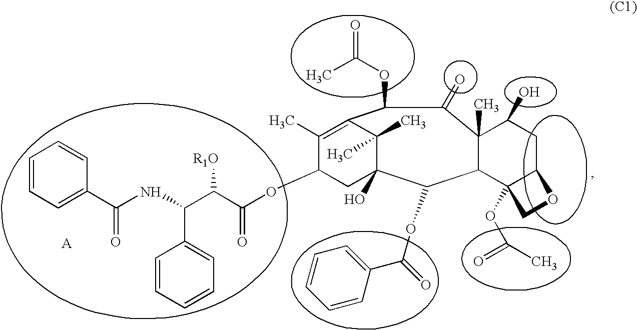

wherein the gray-highlighted portions may be substituted and the non-highlighted portion is the taxane core. A side-chain (labeled “A” in the diagram) is desirably present in order for the compound to have good activity as a cell cycle inhibitor. Examples of compounds having this structure include paclitaxel (Merck Index entry 7117), docetaxol (TAXOTERE, Merck Index entry 3458), and 3′-desphenyl-3′-(4-nitrophenyl)-N-debenzoyl-N-(t-butoxycarbonyl)-10-deacetyltaxol.

In one aspect, suitable paclitaxel-like compounds are disclosed in U.S. Pat. No. 5,440,056 as having the structure (C2):

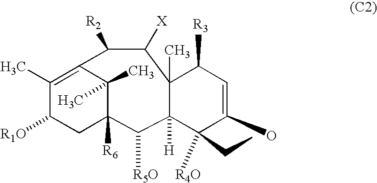

wherein X may be oxygen (paclitaxel), hydrogen (9-deoxy derivatives), thioacyl, or dihydroxyl precursors; R₁ is selected from paclitaxel or TAXOTERE side chains or alkanoyl of the formula (C3)

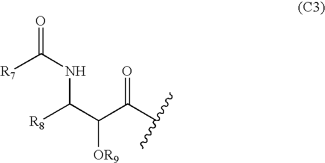

wherein R₇ is selected from hydrogen, alkyl, phenyl, alkoxy, amino, phenoxy (substituted or unsubstituted); R₈ is selected from hydrogen, alkyl, hydroxyalkyl, alkoxyalkyl, aminoalkyl, phenyl (substituted or unsubstituted), alpha or beta-naphthyl; and R₉ is selected from hydrogen, alkanoyl, substituted alkanoyl, and aminoalkanoyl; where substitutions refer to hydroxyl, sulfhydryl, allalkoxyl, carboxyl, halogen, thioalkoxyl, N,N-dimethylamino, alkylamino, dialkylamino, nitro, and —OSO₃H, and/or may refer to groups containing such substitutions; R₂ is selected from hydrogen or oxygen-containing groups, such as hydrogen, hydroxyl, alkoyl, alkanoyloxy, aminoalkanoyloxy, and peptidylalkanoyloxy; R₃ is selected from hydrogen or oxygen-containing groups, such as hydrogen, hydroxyl, alkoyl, alkanoyloxy, aminoalkanoyloxy, and peptidylalkanoyloxy, and may further be a silyl containing group or a sulphur containing group; R₄ is selected from acyl, alkyl, alkanoyl, aminoalkanoyl, peptidylalkanoyl and aroyl; R₅ is selected from acyl, alkyl, alkanoyl, aminoalkanoyl, peptidylalkanoyl and aroyl; R₆ is selected from hydrogen or oxygen-containing groups, such as hydrogen, hydroxyl alkoyl, alkanoyloxy, aminoalkanoyloxy, and peptidylalkanoyloxy.

Paclitaxel-like compounds are also disclosed in PCT International Patent Application No. WO 93/10076. As disclosed in this publication, the compound should have a side chain attached to the taxane nucleus at C₁₃, as shown in the structure below (formula C4), in order to confer antitumor activity to the taxane.

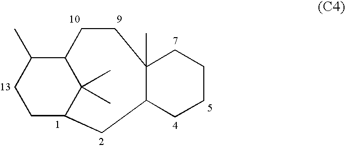

WO 93/10076 discloses that the taxane nucleus may be substituted at any position with the exception of the existing methyl groups. The substitutions may include, for example, hydrogen, alkanoyloxy, alkenoyloxy, aryloyloxy. In addition, oxo groups may be attached to carbons labeled 2, 4, 9, and/or 10. As well, an oxetane ring may be attached at carbons 4 and 5. As well, an oxirane ring may be attached to the carbon labeled 4.

In one aspect, the paclitaxel-like compound is disclosed in U.S. Pat. No. 5,440,056, which discloses 9-deoxo taxanes. These are compounds lacking an oxo group at the carbon labeled 9 in the taxane structure shown above (formula C4). The taxane ring may be substituted at the carbons labeled 1, 7 and 10 (independently) with H, OH, O—R, or O—CO—R where R is an alkyl or an aminoalkyl. As well, it may be substituted at carbons labeled 2 and 4 (independently) with aryol, alkanoyl, aminoalkanoyl or alkyl groups. The side chain of formula (C3) may be substituted at R₇ and R₈ (independently) with phenyl rings, substituted phenyl rings, linear alkanes/alkenes, and groups containing H, O or N. R₉ may be substituted with H, or a substituted or unsubstituted alkanoyl group.

Additional examples of paclitaxel-like compounds which may be used include the paclitaxel derivatives described in U.S. Ser. No. 11/357,368, entitled, “Stents combined with paclitaxel derivatives,” filed Feb. 17, 2006.

In one aspect of this disclosure, the paclitaxel-like compound is anti-angiogenic as determined by the CAM assay.

Dipyridamole is also known as (2-{[9-(bis(2-hydroxyethyl)amino)-2,7-bis(1-piperidyl)-3,5,8,10-tetrazabicyclo[4.4.0]deca-2,4,7,9,11-pentaen-4-yl]-(2-hydroxyethyl)amino}ethanol and is also referred to as 2,6-bis(diethanolamino)-4,8-dipiperidinopyrimido(5,4-d) pyrimidine). Dipyridamole has the following chemical structure:

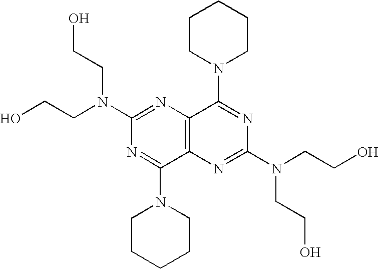

In certain aspects, the present disclosure contemplates the use of at least one dipyridamole derivative or analogue. In one embodiment, medical devices are provided that include a combination of paclitaxel (or an analogue or derivative thereof) and a dipyridamole derivative or analogue. Examples of dipyridamole analogues and derivatives include RA-233 (mopidamol, AR-102, OLX-102, Rapenton) (2,6-bis(diethylamino)-4-piperidinopyrimido[5,4d]pyrimidine); R-E 244 (4-(ethanolisopropanolamino)-2,7-di-(2′-methylmorpholino)-6-phenylpterine); and Rx-RA85, 4-(1-oxidothiomorpholino)-8-phenethylthio-2-piperazino-pyrimido(5,4-d)pyrimidine; dipyridamole monoacetate; NU3026 (2,6-di-(2,2-dimethyl-1,3-dioxolan-4-yl)-methoxy-4,8-di-piperidinopyrimidopyrimidine); NU3059 (2,6-bis(2,3-dimethoxypropoxy)-4,8-di-piperidinopyrimidopyrimidine); NU3060 (2,6bis[N,N-di(2-methoxy)ethyl]-4,6-di-piperidinopyrimidopyrimidine); NU3076 (2,6-bis(diethanolamino)-4,8-di-4-methoxybenzylaminopyrimidopyrimidine); BIBW22BS (CAS 137694-16-7) 2-propanol, 1-((2,7-bis((2R,6S)-2,6-dimethyl-4-morpholinyl)-6-phenyl-4-pteridinyl)(2-hydroxyethyl)amino)-2-methyl-, rel-); BIBW022 (CAS137694-16-7) 2-propanol, 1-((2,7-bis(2,6-dimethyl-4-morpholinyl)-6-phenyl-4-pteridinyl)(2-hydroxyethyl)amino)-2-methyl-, (cis(cis))-; VK-774 (CAS 33548-44-6) thieno(3,2-d)pyrimidine, 4-(4-morpholinyl)-2-(1-piperazinyl)-, dihydrochlorid; and RA-642 (2,2′-[(4,8-bis(diethylamino)-pyrimido[5,4-d]pyrimidine-2,6-diyl)di-(2-methoxyethyl)imino]diethanol). Additional examples of dipyridamole analogues and derivatives for use in this disclosure are described in, e.g., J. Brazilian Chemical Society (1995), 6(2), 111-18 and J. Biomater. Sci. Polymer Edn. (1991), 2(1), 37-52.

C. Association of Compounds with a Device

In the practice of this disclosure, the compounds paclitaxel and dipyridamole, or analogues or derivatives thereof (such as those described above), are associated with a medical device or a medical implant (collectively a “medical device” or “device”). There are numerous methods available for associating the compounds with the device or implant, including those described below. Worth noting are two of the preferred options, which are (1) to affix the compounds to the device in a manufacturing setting, such that transport of the device results in simultaneous transport of the compounds; (2) to provide a composition comprising the two compounds, where that composition is not physically attached to the device, but where that composition is delivered to the site in the patient where the device is, or will be, situated, and optionally thereafter physically connecting the device and composition Also worth noting as an initial matter is that the compounds need not be directly associated with one another, i.e., paclitaxel might be associated with one region of the device while dipyridamole is associated with a different region of the device.

1) Systemic, Regional and Local Delivery

A variety of delivery technologies are available for systemic, regional and local delivery of compounds, in order to provide elevated levels of compounds in the vicinity of the device, including: (a) using drug-delivery catheters for local, regional or systemic delivery of compounds to the tissue surrounding the device (typically, drug delivery catheters are advanced through the circulation or inserted directly into tissues under radiological guidance until they reach the desired anatomical location; the compound can then be released from the catheter lumen in high local concentrations in order to deliver desired doses of the compound to the tissue surrounding the device); (b) drug localization techniques such as magnetic, ultrasonic or MRI-guided drug delivery; (c) chemical modification of the compound or formulation designed to increase uptake of the compound into the targeted tissues (e.g., antibodies directed against damaged or healing tissue components such as macrophages, neutrophils, smooth muscle cells, fibroblasts, extracellular matrix components, neovascular tissue); (d) chemical modification of the compounds or formulation designed to localize the compound to areas of bleeding or disrupted vasculature; (e) direct injection of the compound, for example, under endoscopic vision; (0 administration of the compounds via angioplasty balloons or other specialized drug delivery balloons such as “sweaty” balloons, microinjector balloons or other intravascular devices designed to deliver the drug into or around the vasculature; and/or (g) administration of the compounds described herein to the surface of the body passageway such as via “endoluminal paving” techniques.

2) Sustained-Release Preparations

The compounds may be admixed with, blended with, conjugated to, or otherwise modified to contain a polymer composition (which may be either biodegradable or non-biodegradable) or a non-polymeric composition in order to release the compounds over a prolonged period of time. For many of the intended uses of the compounds, localized delivery as well as localized sustained delivery of the compounds may be required. For example, the compounds may be formed into a composition in order to provide for their release over a period of time.

Representative examples of biodegradable polymers suitable for the delivery of the compounds include albumin, collagen, gelatin, hyaluronic acid, starch, cellulose and cellulose derivatives (e.g., methylcellulose, hydroxypropylcellulose, hydroxypropylmethylcellulose, carboxymethylcellulose, cellulose acetate phthalate, cellulose acetate succinate, hydroxypropylmethylcellulose phthalate), casein, dextrans, polysaccharides, fibrinogen, poly(ether ester) multiblock copolymers, based on poly(ethylene glycol) and poly(butylene terephthalate), tyrosine-derived polycarbonates (e.g., U.S. Pat. No. 6,120,491), poly(hydroxyl acids), poly(D,L-lactide), poly(D,L-lactide-co-glycolide), poly(glycolide), poly(hydroxybutyrate), polydioxanone, poly(alkylcarbonate) and poly(orthoesters), polyesters, poly(hydroxyvaleric acid), polydioxanone, degradable polyesters, poly(malic acid), poly(tartronic acid), poly(acrylamides), polyanhydrides, polyphosphazenes, poly(amino acids), poly(alkylene oxide)-poly(ester) block copolymers (e.g., X—Y, X—Y—X or Y—X—Y, R—(Y—X)ₙ, R—(X—Y) where X is a polyalkylene oxide and Y is a polyester (e.g., polyester can comprise the residues of one or more of the monomers selected from lactide, lactic acid, glycolide, glycolic acid, □-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one), R is a multifunctional initiator and copolymers as well as blends thereof) and the copolymers as well as blends thereof (see generally, Ilium, L., Davids, S. S. (eds.) “Polymers in Controlled Drug Delivery” Wright, Bristol, 1987; Arshady, J. Controlled Release 17:1-22, 1991; Pitt, Int. J. Phar. 59:173-196, 1990; Holland et al., J. Controlled Release 4:155-0180, 1986).

Representative examples of non-degradable polymers suitable for the delivery of compounds include poly(ethylene-co-vinyl acetate) (“EVA”) copolymers, non-degradable polyesters, such as poly(ethylene terephthalate), silicone rubber, acrylic polymers (polyacrylate, polyacrylic acid, polymethylacrylic acid, polymethylmethacrylate, poly(butyl methacrylate)), poly(alkylcynoacrylate) (e.g., poly(ethylcyanoacrylate), poly(butylcyanoacrylate) poly(hexylcyanoacrylate) poly(octylcyanoacrylate)), acrylic resin, polyethylene, polypropylene, polyamides (nylon 6,6), polyurethanes (e.g., CHRONOFLEX AR, CHRONOFLEX AL, BIONATE, and PELLETHANE), poly(ester urethanes), poly(ether urethanes), poly(ester-urea), cellulose esters (e.g., nitrocellulose), polyethers (poly(ethylene oxide), poly(propylene oxide), polyoxyalkylene ether block copolymers based on ethylene oxide and propylene oxide such as the PLURONIC polymers (e.g., F-127 or F87) from BASF Corporation (Mount Olive, N.J.), and poly(tetramethylene glycol), styrene-based polymers (polystyrene, poly(styrene sulfonic acid), poly(styrene)-block-poly(isobutylene)-block-poly(styrene), poly(styrene)-poly(isoprene) block copolymers), and vinyl polymers (polyvinylpyrrolidone, poly(vinyl alcohol), poly(vinyl acetate phthalate) as well as copolymers and blends thereof. Polymers may also be developed which are either anionic (e.g., alginate, carrageenan, carboxymethyl cellulose, poly(acrylamido-2-methyl propane sulfonic acid) and copolymers thereof, poly(methacrylic acid and copolymers thereof and poly(acrylic acid) and copolymers thereof, as well as blends thereof, or cationic (e.g., chitosan, poly-L-lysine, polyethylenimine, and poly(allyl amine)) and blends, copolymers and branched polymers thereof (see generally, Dunn et al., J. Applied Polymer Sci. 50:353-365, 1993; Cascone et al., J. Materials Sci.: Materials in Medicine 5:770-774, 1994; Shiraishi et al., Biol. Pharm. Bull. 16(11):1164-1168, 1993; Thacharodi and Rao, Int'l J. Pharm. 120:115-118, 1995; Miyazaki et al., Intl J. Pharm. 118:257-263, 1995).

Particularly preferred polymers include poly(ethylene-co-vinyl acetate), polyurethanes (e.g., CHRONOFLEX AR, CHRONOFLEX AL, BIONATE, and PELLETHANE), poly(D,L-lactic acid) oligomers and polymers, poly(L-lactic acid) oligomers and polymers, poly(glycolic acid), copolymers of lactic acid and glycolic acid, poly(caprolactone), poly(valerolactone), polyanhydrides, copolymers of poly(caprolactone) or poly(lactic acid) with a polyethylene glycol (e.g., MePEG), poly(alkylene oxide)-poly(ester) block copolymers (e.g., X—Y, X—Y—X or Y—X—Y, R—(Y—X)ₙ, R—(X—Y)ₙ where X is a polyalkylene oxide and Y is a polyester (e.g., polyester can comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one), R is a multifunctional initiator and copolymers as well as blends thereof), nitrocellulose, silicone rubbers, poly(styrene)block-poly(isobutylene)-block-poly(styrene), poly(acrylate) polymers and blends, admixtures, or co-polymers of any of the above. Other preferred polymers include collagen, poly(alkylene oxide)-based polymers, polysaccharides such as hyaluronic acid, chitosan and fucans, and copolymers of polysaccharides with degradable polymers, as well as blends thereof.

Other representative polymers capable of sustained localized delivery of compounds include carboxylic polymers, polyacetates, polycarbonates, polyethers, polyethylenes, polyvinylbutyrals, polysilanes, polyureas, polyoxides, polystyrenes, polysulfides, polysulfones, polysulfonides, polyvinylhalides, pyrrolidones, rubbers, thermal-setting polymers, cross-linkable acrylic and methacrylic polymers, ethylene acrylic acid copolymers, styrene acrylic copolymers, vinyl acetate polymers and copolymers, vinyl acetal polymers and copolymers, epoxies, melamines, other amino resins, phenolic polymers, and copolymers thereof, water-insoluble cellulose ester polymers (including cellulose acetate propionate, cellulose acetate, cellulose acetate butyrate, cellulose nitrate, cellulose acetate phthalate, and mixtures thereof), polyvinylpyrrolidone, polyethylene glycols, polyethylene oxide, polyvinyl alcohol, polyethers, polysaccharides, hydrophilic polyurethane, polyhydroxyacrylate, dextran, xanthan, hydroxypropyl cellulose, and homopolymers and copolymers of N-vinylpyrrolidone, N-vinyllactam, N-vinyl butyrolactam, N-vinyl caprolactam, other vinyl compounds having polar pendant groups, acrylate and methacrylate having hydrophilic esterifying groups, hydroxyacrylate, and acrylic acid, and combinations thereof; cellulose esters and ethers, ethyl cellulose, hydroxyethyl cellulose, cellulose nitrate, cellulose acetate, cellulose acetate butyrate, cellulose acetate propionate, natural and synthetic elastomers, rubber, acetal, styrene polybutadiene, acrylic resin, polyvinylidene chloride, polycarbonate, homopolymers and copolymers of vinyl compounds, polyvinylchloride, and polyvinylchloride acetate.

Representative examples of patents relating to drug-delivery polymers and their preparation, which may be utilized in the composition of the present disclosure, include PCT Publication Nos. WO 98/19713, WO 01/17575, WO 01/41821, WO 01/41822, and WO 01/15526 (as well as the corresponding U.S. applications); U.S. Pat. Nos. 4,500,676, 4,582,865, 4,629,623, 4,636,524, 4,713,448, 4,795,741, 4,913,743, 5,069,899, 5,099,013, 5,128,326, 5,143,724, 5,153,174, 5,246,698, 5,266,563, 5,399,351, 5,525,348, 5,800,412, 5,837,226, 5,942,555, 5,997,517, 6,007,833, 6,071,447, 6,090,995, 6,106,473, 6,110,483, 6,121,027, 6,156,345, 6,214,901, 6,368,611 6,630,155, 6,528,080, RE37,950, 6,46,1631, 6,143,314, 5,990,194, 5,792,469, 5,780,044, 5,759,563, 5,744,153, 5,739,176, 5,733,950, 5,681,873, 5,599,552, 5,340,849, 5,278,202, 5,278,201, 6,589,549, 6,287,588, 6,201,072, 6,117,949, 6,004,573, 5,702,717, 6,413,539, 5,714,159, 5,612,052; and U.S. Patent Application Publication Nos. 2003/0068377, 2002/0192286, 2002/0076441, and 2002/0090398.

In one embodiment, all or a portion of the device is coated with a primer (bonding) layer and a drug release layer, as described in U.S. patent application entitled, “Stent with Medicated Multi-Layer Hybrid Polymer Coating,” filed Sep. 16, 2003 (U.S. Ser. No. 10/662,877). Other examples of coating including those described in PCT Publication No. WO 92/00747; and U.S. Pat. Nos. 6,110,483 and 6,368,611.

The polymeric composition can be fashioned in a variety of forms, with desired release characteristics and/or with specific properties depending upon the device, composition or implant being utilized. For example, polymeric carriers may be fashioned to release a compound upon exposure to a specific triggering event such as pH (see, e.g., Heller et al., “Chemically Self-Regulated Drug Delivery Systems,” in Polymers in Medicine III, Elsevier Science Publishers B.V., Amsterdam, 1988, pp. 175-188; Kang et al., J. Applied Polymer Sci. 48:343-354, 1993; Dong et al., J. Controlled Release 19:171-178, 1992; Dong and Hoffman, J. Controlled Release 15:141-152, 1991; Kim et al., J. Controlled Release 28:143-152, 1994; Cornejo-Bravo et al., J. Controlled Release 33:223-229, 1995; Wu and Lee, Pharm. Res. 10(10):1544-1547, 1993; Serres et al., Pharm. Res. 13(2):196-201, 1996; Peppas, “Fundamentals of pH- and Temperature-Sensitive Delivery Systems,” in Gurny et al. (eds.), Pulsatile Drug Delivery, Wissenschaftliche Verlagsgesellschaft mbH, Stuttgart, 1993, pp. 41-55; Doelker, “Cellulose Derivatives,” 1993, in Peppas and Langer (eds.), Biopolymers I, Springer-Verlag, Berlin). Representative examples of pH-sensitive polymers include poly (acrylic acid) and its derivatives (including for example, homopolymers such as poly(aminocarboxylic acid); poly(acrylic acid); poly(methyl acrylic acid), copolymers of such homopolymers, and copolymers of poly(acrylic acid) and/or acrylate or acrylamide lmonomers such as those discussed above. Other pH sensitive polymers include polysaccharides such as cellulose acetate phthalate; hydroxypropylmethylcellulose phthalate; hydroxypropylmethylcellulose acetate succinate; cellulose acetate trimellilate; and chitosan. Yet other pH sensitive polymers include any mixture of a pH sensitive polymer and a water-soluble polymer.

Likewise, compounds can be delivered via polymeric carriers which are temperature sensitive (see, e.g., Chen et al., “Novel Hydrogels of a Temperature-Sensitive PLURONIC Grafted to a Bioadhesive Polyacrylic Acid Backbone for Vaginal Drug Delivery,” in Proceed. Intern. Symp. Control. Rel. Bioact. Mater. 22:167-168, Controlled Release Society, Inc., 1995; Okano, “Molecular Design of Stimuli-Responsive Hydrogels for Temporal Controlled Drug Delivery,” in Proceed. Intern. Symp. Control. Rel. Bioact. Mater. 22:111-112, Controlled Release Society, Inc., 1995; Johnston et al., Pharm. Res. 9(3):425-433, 1992; Tung, Int'l J. Pharm. 107:85-90, 1994; Harsh and Gehrke, J. Controlled Release 17:175-186, 1991; Bae et al., Pharm. Res. 8(4):531-537, 1991; Dinarvand and D'Emanuele, J. Controlled Release 36:221-227, 1995; Yu and Grainger, “Novel Thermo-sensitive Amphiphilic Gels: Poly N-isopropylacrylamide-co-sodium acrylate-co-n-N-alkylacrylamide Network Synthesis and Physicochemical Characterization,” Dept. of Chemical & Biological Sci., Oregon Graduate Institute of Science & Technology, Beaverton, Oreg., pp. 820-821; Zhou and Smid, “Physical Hydrogels of Associative Star Polymers,” Polymer Research Institute, Dept. of Chemistry, College of Environmental Science and Forestry, State Univ. of New York, Syracuse, N.Y., pp. 822-823; Hoffman et al., “Characterizing Pore Sizes and Water ‘Structure’ in Stimuli-Responsive Hydrogels,” Center for Bioengineering, Univ. of Washington, Seattle, Wash., p. 828; Yu and Grainger, “Thermo-sensitive Swelling Behavior in Crosslinked N-isopropylacrylamide Networks: Cationic, Anionic and Ampholytic Hydrogels,” Dept. of Chemical & Biological Sci., Oregon Graduate Institute of Science & Technology, Beaverton, Oreg., pp. 829-830; Kim et al., Pharm. Res. 9(3):283-290, 1992; Bae et al., Pharm. Res. 8(5):624-628, 1991; Kono et al., J. Controlled Release 30:69-75, 1994; Yoshida et al., J. Controlled Release 32:97-102, 1994; Okano et al., J. Controlled Release 36:125-133, 1995; Chun and Kim, J. Controlled Release 38:39-47, 1996; D'Emanuele and Dinarvand, Int'l J. Pharm. 118:237-242, 1995; Katono et al., J. Controlled Release 16:215-228, 1991; Hoffman, “Thermally Reversible Hydrogels Containing Biologically Active Species,” in Migliaresi et al. (eds.), Polymers in Medicine III, Elsevier Science Publishers B.V., Amsterdam, 1988, pp. 161-167; Hoffman, “Applications of Thermally Reversible Polymers and Hydrogels in Therapeutics and Diagnostics,” in Third International Symposium on Recent Advances in Drug Delivery Systems, Salt Lake City, Utah, Feb. 24-27, 1987, pp. 297-305; Gutowska et al., J. Controlled Release 22:95-104, 1992; Palasis and Gehrke, J. Controlled Release 18:1-12, 1992; Paavola et al., Pharm. Res. 12(12):1997-2002, 1995).

Representative examples of thermogelling polymers, and the gelatin temperature (LCST (° C.)) include homopolymers such as poly(N-methyl-N-n-propylacrylamide), 19.8; poly(N-n-propylacrylamide), 21.5; poly(N-methyl-N-isopropylacrylamide), 22.3; poly(N-n-propylmethacrylamide), 28.0; poly(N-isopropylacrylamide), 30.9; poly(N, n-diethylacrylamide), 32.0; poly(N-isopropylmethacrylamide), 44.0; poly(N-cyclopropylacrylamide), 45.5; poly(N-ethylmethyacrylamide), 50.0; poly(N-methyl-N-ethylacrylamide), 56.0; poly(N-cyclopropylmethacrylamide), 59.0; poly(N-ethylacrylamide), 72.0. Moreover thermogelling polymers may be made by preparing copolymers between (among) monomers of the above, or by combining such homopolymers with other water-soluble polymers such as acrylmonomers (e.g., acrylic acid and derivatives thereof, such as methylacrylic acid, acrylate monomers and derivatives thereof, such as butyl methacrylate, butyl acrylate, lauryl acrylate, and acrylamide monomers and derivatives thereof, such as N-butyl acrylamide and acrylamide).

Other representative examples of thermogelling polymers include cellulose ether derivatives such as hydroxypropyl cellulose, 41° C.; methyl cellulose, 55° C.; hydroxypropylmethyl cellulose, 66° C.; and ethylhydroxyethyl cellulose, polyalkylene oxide-polyester block copolymers of the structure X—Y, Y—X—Y and X—Y—X where X in a polyalkylene oxide and Y is a biodegradable polyester (e.g., PLG-PEG-PLG) and PLURONICs such as F-127, 10-15° C.; L-122, 19° C.; L-92, 26° C.; L-81, 20° C.; and L-61, 24° C.

Representative examples of patents relating to thermally gelling polymers and the preparation include U.S. Pat. Nos. 6,451,346; 6,201,072; 6,117,949; 6,004,573; 5,702,717; and 5,484,610; and PCT Publication Nos. WO 99/07343; WO 99/18142; WO 03/17972; WO 01/82970; WO 00/18821; WO 97/15287; WO 01/41735; WO 00/00222 and WO 00/38651.

The compounds may be linked by occlusion in the matrices of the polymer, bound by covalent linkages, or encapsulated in microcapsules. Within certain embodiments of this disclosure, compositions are provided in non-capsular formulations such as microspheres (ranging from nanometers to micrometers in size), pastes, threads of various size, films, or sprays. In one aspect, one or both of the compounds may be incorporated into biodegradable magnetic nanospheres. The nanospheres may be used, for example, to replenish one or both of the compounds into an implanted intravascular device, such as a stent containing a weak magnetic alloy (see, e.g., Z. Forbes, B. B. Yellen, G. Friedman, K. Barbee. “An approach to targeted drug delivery based on uniform magnetic fields,” IEEE Trans. Magn. 39(5): 3372-3377 (2003)).

Within certain aspects of the present disclosure, compositions may be fashioned in the form of microspheres, microparticles and/or nanoparticles having any size ranging from about 30 nm to 500 μm, depending upon the particular use. These compositions can be formed by spray-drying methods, milling methods, coacervation methods, W/O emulsion methods, W/O/W emulsion methods, and solvent evaporation methods. In other aspects, these compositions can include microemulsions, emulsions, liposomes and micelles. Alternatively, such compositions may also be readily applied as a “spray”, which solidifies into a film or coating for use as a device/implant surface coating or to line the tissues of the implantation site. Such sprays may be prepared from microspheres of a wide array of sizes, including for example, from 0.1 μm to 3 from 10 μm to 30 μm, and from 30 μm to 100 μm.

Compositions of the present disclosure may also be prepared in a variety of “paste” or gel forms. For example, compositions are provided which are liquid at one temperature (e.g., temperature greater than 37° C., such as 40° C., 45° C., 50° C., 55° C. or 60° C.), and solid or semi-solid at another temperature (e.g., ambient body temperature, or any temperature lower than 37° C.). Such “thermopastes” may be readily made utilizing a variety of techniques (see, e.g., PCT Publication WO 98/24427). Other pastes may be applied as a liquid, which solidify in vivo due to dissolution of a water-soluble component of the paste and precipitation of encapsulated drug into the aqueous body environment. These “pastes” and “gels” containing compounds are particularly useful for application to the surface of tissues that will be in contact with the device.

Within yet other aspects of this disclosure, the compositions may be formed as a film or tube. These films or tubes can be porous or non-porous. Preferably, such films or tubes are generally less than 5, 4, 3, 2, or 1 mm thick, more preferably less than 0.75 mm, 0.5 mm, 0.25 mm, or, 0.10 mm thick. Films or tubes can also be generated of thicknesses less than 50 μm, 25 μm or 10 μm. Such films are preferably flexible with a good tensile strength (e.g., greater than 50, preferably greater than 100, and more preferably greater than 150 or 200 N/cm²), good adhesive properties (i.e., adheres to moist or wet surfaces), and have controlled permeability. Compounds contained in polymeric films are particularly useful for application to the surface of a device as well as to the surface of tissue, cavity or an organ.

Within further aspects of the present disclosure, polymeric carriers are provided which are adapted to contain and release a hydrophobic compound, and/or the carrier containing the hydrophobic compound in combination with a carbohydrate, protein or polypeptide. Within certain embodiments, the polymeric carrier contains or comprises regions, pockets, or granules of one or more hydrophobic compounds. For example, within one embodiment of this disclosure, hydrophobic compounds may be incorporated within a matrix which contains the hydrophobic compound, followed by incorporation of the matrix within the polymeric carrier. A variety of matrices can be utilized in this regard, including for example, carbohydrates and polysaccharides such as starch, cellulose, dextran, methylcellulose, sodium alginate, heparin, chitosan and hyaluronic acid, proteins or polypeptides such as albumin, collagen and gelatin. Within alternative embodiments, hydrophobic compounds may be contained within a hydrophobic core, and this core contained within a hydrophilic shell.

Other carriers that may likewise be utilized to contain and deliver compounds described herein include: hydroxypropyl cyclodextrin (Cserhati and Hollo, Int. J. Pharm. 108:69-75, 1994), liposomes (see, e.g., Sharma et al., Cancer Res. 53:5877-5881, 1993; Sharma and Straubinger, Pharm. Res. 11(60):889-896, 1994; WO 93/18751; U.S. Pat. No. 5,242,073), liposome/gel (WO 94/26254), nanocapsules (Bartoli et al., J. Microencapsulation 7(2):191-197, 1990), micelles (Alkan-Onyuksel et al., Pharm. Res. 11(2):206-212, 1994), implants (Jampel et al., Invest. Ophthalm. Vis. Science 34(11):3076-3083, 1993; Walter et al., Cancer Res. 54:22017-2212, 1994), nanoparticles (Violante and Lanzafame PAACR), nanoparticles—modified (U.S. Pat. No. 5,145,684), nanoparticles (surface modified) (U.S. Pat. No. 5,399,363), micelle (surfactant) (U.S. Pat. No. 5,403,858), synthetic phospholipid compounds (U.S. Pat. No. 4,534,899), gas borne dispersion (U.S. Pat. No. 5,301,664), liquid emulsions, foam, spray, gel, lotion, cream, ointment, dispersed vesicles, particles or droplets solid- or liquid-aerosols, microemulsions (U.S. Pat. No. 5,330,756), polymeric shell (nano- and micro-capsule) (U.S. Pat. No. 5,439,686), emulsion (Tarr et al., Pharm Res. 4: 62-165, 1987), nanospheres (Hagan et al., Proc. Intern. Symp. Control Rel. Bioact. Mater. 22, 1995; Kwon et al., Pharm Res. 12(2):192-195; Kwon et al., Pharm Res. 10(7):970-974; Yokoyama et al., J. Contr. Rel. 32:269-277, 1994; Gref et al., Science 263:1600-1603, 1994; Bazile et al., J. Pharm. Sci. 84:493-498, 1994) and implants (U.S. Pat. No. 4,882,168).

Within another aspect of the present disclosure, polymeric carriers can be materials that are formed in situ. In one embodiment, the precursors can be monomers or macromers that contain unsaturated groups that can be polymerized and/or cross-linked. The monomers or macromers can then, for example, be injected into the treatment area or onto the surface of the treatment area and polymerized in situ using a radiation source (e.g., visible or UV light) or a free radical system (e.g., potassium persulfate and ascorbic acid or iron and hydrogen peroxide). The polymerization step can be performed immediately prior to, simultaneously to or post injection of the reagents into the treatment site. Representative examples of compositions that undergo free radical polymerization reactions are described in WO 01/44307, WO 01/68720, WO 02/072166, WO 03/043552, WO 93/17669, WO 00/64977; U.S. Pat. Nos. 5,900,245, 6,051,248, 6,083,524, 6,177,095, 6,201,065, 6,217,894, 6,639,014, 6,352,710, 6,410,645, 6,531,147, 5,567,435, 5,986,043, 6,602,975; U.S. Patent Application Publication Nos. 2002/012796A1, 2002/0127266A1, 2002/0151650A1, 2003/0104032A1, 2002/0091229A1, and 2003/0059906A1.

In another embodiment, the reagents can undergo an electrophilic-nucleophilic reaction to produce a crosslinked matrix. For example, a 4-armed thiol derivatized polyethylene glycol can be reacted with a 4 armed NHS-derivatized polyethylene glycol under basic conditions (pH>about 8). Representative examples of compositions that undergo electrophilic-nucleophilic crosslinking reactions are described in U.S. Pat. Nos. 5,752,974; 5,807,581; 5,874,500; 5,936,035; 6,051,648; 6,165,489; 6,312,725; 6,458,889; 6,495,127; 6,534,591; 6,624,245; 6,566,406; 6,610,033; 6,632,457; PCT Application Published Nos. WO 04/060405 and WO 04/060346. Other examples of in situ forming materials that can be used include those based on the crosslinking of proteins (described in U.S. Pat. Nos. RE38158; 4,839,345; 5,514,379, 5,583,114; 6,458,147; 6,371,975; U.S. Publication Nos 2002/0161399; 2001/0018598 and PCT Publication Nos. WO 03/090683; WO 01/45761; WO 99/66964 and WO 96/03159).

In addition to the compositions and methods described above, there are various other compositions and methods that are known in the art. Representative examples of these compositions and methods for applying (e.g., coating) these compositions to devices are described in U.S. Pat. Nos. 6,610,016; 6,358,557; 6,306,176; 6,110,483; 6,106,473; 5,997,517; 5,800,412; 5,525,348; 5,331,027; 5,001,009; 6,562,136; 6,406,754; 6,344,035; 6,254,921; 6,214,901; 6,077,698; 6,603,040; 6,278,018; 6,238,799; 6,096,726, 5,766,158, 5,599,576, 4,119,094; 4,100,309; 6,599,558; 6,369,168; 6,521,283; 6,497,916; 6,251,964; 6,225,431; 6,087,462; 6,083,257; 5,739,237; 5,739,236; 5,705,583; 5,648,442; 5,645,883; 5,556,710; 5,496,581; 4,689,386; 6,214,115; 6,090,901; 6,599,448; 6,054,504; 4,987,182; 4,847,324; and 4,642,267; U.S. Patent Application Publication Nos. 2002/0146581, 2003/0129130, 2003/0129130, 2001/0026834; 2003/0190420; 2001/0000785; 2003/0059631; 2003/0190405; 2002/0146581; 2003/020399; 2001/0026834; 2003/0190420; 2001/0000785; 2003/0059631; 2003/0190405; and 2003/020399; and PCT Publication Nos. WO 02/055121; WO 01/57048; WO 01/52915; and WO 01/01957.

Within another aspect of this disclosure, the compound(s) can be delivered with a non-polymeric agent. These non-polymeric carriers can include sucrose derivatives (e.g., sucrose acetate isobutyrate, sucrose oleate), sterols such as cholesterol, stigmasterol, β-sitosterol, and estradiol; cholesteryl esters such as cholesteryl stearate; C₁₂-C₂₄ fatty acids such as lauric acid, myristic acid, palmitic acid, stearic acid, arachidic acid, behenic acid, and lignoceric acid; C₁₈-C₃₆ mono-, di- and triacylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate and mixtures thereof; sucrose fatty acid esters such as sucrose distearate and sucrose palmitate; sorbitan fatty acid esters such as sorbitan monostearate, sorbitan monopalmitate and sorbitan tristearate; C₁₆-C₁₈ fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine (lecithin), phosphatidylserine, phosphatidylethanolamine, phosphatidylinositol, and lysoderivatives thereof; sphingosine and derivatives thereof; sphingomyelins such as stearyl, palmitoyl, and tricosanyl sphingomyelins; ceramides such as stearyl and palmitoyl ceramides; glycosphingolipids; lanolin and lanolin alcohols, calcium phosphate, sintered and unscintered hydroxyapatite, zeolites; and combinations and mixtures thereof. Representative examples of patents relating to non-polymeric delivery systems and the preparation include U.S. Pat. Nos. 5,736,152; 5,888,533; 6,120,789; 5,968,542; and 5,747,058.

The compounds may be delivered as a solution. The compounds can be incorporated directly into the solution to provide a homogeneous solution or dispersion. In certain embodiments, the solution is an aqueous solution. The aqueous solution may further include buffer salts, as well as viscosity modifying agents (e.g., hyaluronic acid, alginates, carboxymethylcellulose (CMC), and the like). In another aspect of this disclosure, the solution can include a biocompatible solvent, such as ethanol, DMSO, glycerol, PEG-200, PEG-300 or NMP.

Within another aspect of this disclosure, the compound(s) can be formulated into a composition that comprises a secondary carrier. The secondary carrier can be in the form of microspheres (e.g., PLGA, PLLA, PDLLA, PCL, gelatin, polydioxanone, poly(alkylcyanoacrylate)), nanospheres (PLGA, PLLA, PDLLA, PCL, gelatin, polydioxanone, poly(alkylcyanoacrylate)), liposomes, emulsions, microemulsions, micelles (SDS, block copolymers of the form X—Y, X—Y—X or Y—X—Y, R—(Y—X)ₙ, R—(X—Y) where X is a polyalkylene oxide (e.g., poly(ethylene oxide, poly(propylene oxide, block copolymers of poly(ethylene oxide) and poly(propylene oxide) and Y is a polyester (e.g., polyester can comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one), R is a multifunctional initiator and copolymers as well as blends thereof), zeolites or cyclodextrins.

Within another aspect of this disclosure, these compound(s)/secondary carrier compositions can be a) incorporated directly into or onto the device, b) incorporated into a solution, c) incorporated into a gel or viscous solution, d) incorporated into the composition used for coating the device, e) incorporated into or onto the device following coating of the device with a coating composition, and/or (f) infiltrated into the tissue surrounding where the device will be, or has been, inserted.

For example, compound(s)-loaded PLGA microspheres can be incorporated into a polyurethane coating solution which is then coated onto the device. In yet another example, the device can be coated with a polyurethane and then allowed to partially dry such that the surface is still tacky. A particulate form of the compound(s) or compound(s)/secondary carrier can then be applied to all or a portion of the tacky coating after which the device is dried. In yet another example, the device can be coated with one of the coatings described above. A thermal treatment process can then be used to soften the coating, afterwhich the compound(s) or the compound(s)/secondary carrier is applied to the entire device or to a portion of the device (e.g., outer surface)

Within another aspect of this disclosure, the coated device which inhibits or reduces an in vivo fibrotic reaction is further coated with a compound or compositions which delay the release of and/or activity of the compound(s). Representative examples of such agents include biologically inert materials such as gelatin, PLGA/MePEG film, PLA, polyurethanes, silicone rubbers, surfactants, lipids, or polyethylene glycol, as well as biologically active materials such as heparin (e.g., to induce coagulation).

For example, in one embodiment of this disclosure, the compound on the device is top-coated with a physical barrier. Such barriers can include non-degradable materials or biodegradable materials such as gelatin, PLGA/MePEG film, PLA, or polyethylene glycol among others. In one embodiment, the rate of diffusion of the compound(s) in the barrier coat is slower that the rate of diffusion of the compound(s) in the coating layer. In the case of PLGA/MePEG, once the PLGA/MePEG becomes exposed to the bloodstream, the MePEG can dissolve out of the PLGA, leaving channels through the PLGA layer to an underlying layer containing the compound(s), which then can then diffuse into the vessel wall and initiate its biological activity.

In another embodiment of this disclosure, a particulate form of the compound(s) may be coated onto the stent (or any of the devices described below) using a polymer (e.g., PLG, PLA, aor a polyurethane). A second polymer, that dissolves slowly or degrades (e.g., MePEG-PLGA or PLG) and that does not contain the active agent, may be coated over the first layer. Once the top layer dissolves or degrades, it exposes the under coating which allows the compound(s) to be exposed to the treatment site or to be released from the coating.

Within another aspect of this disclosure, the outer layer of the coating of a coated device, which inhibits an in vivo fibrotic response, is further treated to crosslink the outer layer of the coating. This can be accomplished by subjecting the coated device to a plasma treatment process. The degree of crosslinking and nature of the surface modification can be altered by changing the RF power setting, the location with respect to the plasma, the duration of treatment as well as the gas composition introduced into the plasma chamber.

Protection of a biologically active surface can also be utilized by coating the device surface with an inert molecule that prevents access to the active site through steric hindrance, or by coating the surface with an inactive form of the compound, which is later activated. For example, the device can be coated with an enzyme, which causes either release of one or more of the compounds or activates a compound.

In another embodiment, the device is coated with compound(s) and then further coated with a composition that comprises an anticoagulant such as heparin. As the anticoagulant dissolves away, the anticoagulant activity slows or stops, and the newly exposed compound is available to inhibit or reduce fibrosis from occurring in the adjacent tissue.

In another aspect, a class of non-polymeric materials with which the device may be coated are calcium phosphate-based materials. Examples of this class of materials include hydroxyapatite, di- and tri-calcium phosphates, and partially or fully amorphous calcium phosphates. Hydroxyapatite coatings show excellent biocompatibility and ability to be reabsorbed, with no adverse side effects, as hydroxyapatite is a natural product, present in bone or tooth enamel, for example.

Hydroxyapatite ceramic coatings in biomedical applications may be produced on surfaces by thermal or plasma spray methods, for example. Formation of the ceramic surface in this manner typically requires high calcinations temperatures, at least 350°, for example. Coatings produced in this manner are also typically of thicknesses that limit their use to rigid devices that provide a solid support, as flexing may cause the ceramic coating to become damaged, for example, by cracking. An alternative method to thermal coating involves biomimetic deposition of hydroxyapatite films to surfaces at room temperature. Formation of the coating in this process is driven by supersaturation of Ca⁺² and PO₄⁻³, under a pH at which hydroxyapatite is the most stable phase. As the process can be performed near room temperature and the solutions are water-based, the crystalline coatings that form may incorporate the combination of compounds. A limitation of this process is that the deposition rate is slow. However, the rate may be enhanced, when depositing a hydroxyapatite coating on the surface of a metal device, for example, by applying an electric field to the metal. Biomimetic deposition in this manner is typically termed electrochemical deposition. The coating produced in this manner may not bond well to metallic surfaces, such as a metal stent, but bonds strongly to previously deposited consolidated hydroxyapatite coatings. A further alternative for deposition of calcium phosphate films, particularly hydroxyapatite, on surfaces at or near room temperature, allowing impregnation or encapsulation of the compounds, is by means of a calcium phosphate cement process. In this process, fine particles of Ca(OH)₂ and anhydrous monocalcium phosphate are milled and mixed in ethanol, followed by film deposition and impregnation by a solution of sodium phosphate. This process yields a microporous, semi-amorphous hydroxyapatite film suitable for delivering the compounds during resorption of the film. As with the biomimetic deposition described above, the hydroxyapatite film deposited in this manner bonds poorly to metallic surfaces but bonds strongly to previously deposited hydroxyapatite films.

Inclusion of compounds into the hydroxyapatite layer may nevertheless be accomplished by simple impregnation of the sintered, porous hydroxyapatite layer. The compounds may simply absorb to the surface of the porous ceramic. Various porous ceramic materials capable of slow release of active agents have been described.

A sol-gel process for coating an implantable medical device with a calcium phosphate coating has also been described. In this method, a calcium salt precursor is added to a hydrolyzed phosphate precursor to yield a calcium phosphate gel, wherein the phosphate precursor may be, for example, alkyl phosphite or a triethylphosphate, and the calcium precursor may be, for example, a water-soluble calcium salt, such as calcium nitrate. The gel may be coated on the surface of the device by, for example, spraying, dip coating, spin coating, electrophosphoretic coating, or electrochemical coating. The coated device may then be calcined at an appropriate elevated temperature for a pre-determined time to yield a calcium phosphate coating with suitable crystallinity, porosity and bonding characteristics.

Devices may be advantageously coated in this manner with various calcium phosphates, including hydroxyapatite or di-, tri- or tetracalcium phosphate, by controlling the ratio of calcium to phosphate in the sol-gel precursor.

In certain embodiments, a single calcium phosphate ceramic coating layer may be applied. Alternatively, a second layer may be applied on the first layer. In some aspects, the covering may continuously cover an outer surface of the device. In other aspects, the covering may continuously cover the inner surface of the device. I yet other aspects, the covering may continuously cover all surfaces of the device. In certain further aspects, the ceramic layer may be applied discontinuously, covering only portions of the surfaces of the device. Whether applied as a continuous or a discontinuous covering, the may be used to absorb and release one or both of the compounds described elsewhere herein. Further control of release characteristics of compounds from the ceramic-coated devices may be accomplished by overcoating the ceramic coated devices with a polymer layer, using polymers and coating methods as described elsewhere herein.

Further description and representative examples of methods for the preparation of ceramic materials and polymer-ceramic matrix composites and for their use in the coating of devices are included in the following: U.S. Pat. Nos. 5,258,044; 5,055,307; 6,426,114; and 6,730,324; U.S. Patent Application Nos. 2002/0155144; 2006/0134160; and 2006/0199876; and PCT Publication Nos. WO 98/16209; WO 98/43558; and WO 2006/024125.

In another aspect, a medical device may include a plurality of reservoirs within its structure, each reservoir configured to house and protect one or more compounds. The reservoirs may be present as divets, holes, pits or pores in the surface of a device or micropores or channels in the device body. In one aspect, the reservoirs are formed from voids in the structure of the device. The reservoirs may extend only partially through the structure of the device, opening only to one surface. Alternatively, the reservoir may extend through the structure of the device, opening to both surfaces. The reservoirs may house a single type of drug or more than one type of drug, a single drug in different concentrations, or different forms of the same drug. Within a particular reservoir extending through the structure, one drug, concentration or form of drug may be exposed at one surface, while another drug, concentration, or form of a drug may be exposed at the opposing surface. A plurality of drugs may be useful when each may address one of a variety of biological processes involved in the treatment of a particular condition. The drug(s) may be formulated with a carrier (e.g., a polymeric or non-polymeric material) that is loaded into the reservoirs. In one aspect, the drug(s) may be loaded into the reservoirs in the form of a viscous liquid or a paste. In another aspect, the drug(s) may in the form of a dry sheet, from which plugs may be punched and placed into divets or holes in the surface of the device. In yet another aspect, the drug(s) may be formed into dry particles, put into the reservoirs in this form, and a solvent added to partially liquefy and adhere the drug(s) into the reservoir space. In a further aspect, the drug(s) may be loaded into the reservoirs as a liquid and allowed to dry. In yet further aspects, a reservoir of a device may have a gradient of water-soluble drug(s) within a layer in the reservoir. Wetting characteristics of the dried drug(s) may be adjusted by including certain additives to improve or control dissolution of the drug(s) from the reservoir in vivo. The filled reservoir can function as a drug delivery depot, which can release drug over a period of time dependent on the release kinetics of the drug from the carrier. In certain embodiments, the reservoir may be loaded with a plurality of layers. Each layer may include a different drug having a particular amount (dose) of drug, and each layer may have a different composition to further tailor the amount of drug that is released from the substrate. The multi-layered carrier may further include a barrier layer that prevents release of the drug(s). The barrier layer can be used, for example, to control the direction that the drug elutes from the void. Further, one or more protective layers may be included within a reservoir or on part or the entire surface of the device to prevent or limit processes that deactivate or degrade the drug(s). Drug(s) may be placed in a reservoir in such a manner as to achieve a particular delivery profile, which may include zero order, pulsatile, increasing, decreasing, sinusoidal, or some other profile. Reservoirs, as described here, may be present on all or on selected surfaces of a device. Further, reservoirs may be included on all or only a portion of the surface of a device. Examples of medical devices that may have reservoirs as described include stents and wires.

A medical device or a portion thereof may comprise a porous surface for absorption and release of the compounds. The porous surface may be made of a material, such as a polymer or a polymer blend, with a plurality of voids therein. A porous polymer coating may be applied to the surface of a device. A drug may be dissolved or suspended in a solvent to form a drug solution or suspension. An electrode and a stent with a porous polymer coating are placed in the solution or suspension of drug and connected to a power source. When the power source is activated, drug is driven into the void spaces on the porous surface of the device.

In the preparation of drug-coated medical devices with porous coatings, the pores may be created by the addition of solid particles to a mixture comprising a solvent, a drug, and a polymer to make a suspension of the dispersed solid particles. Solid particles may be dispersed by physical agitation or any other method known in the art. Application of the suspension to the surface of the device yields a porous coating, wherein the pores are created by the solid particles that have been added. A surfactant may be added to the suspension to prevent or decrease flocculation of the solid particles, so that the solid particles are substantially uniformly distributed when the coating is applied to the device. The surfactant may be any biologically compatible surfactant, for example, TWEEN 80®, TWEEN 86®, TWEEN 20®, and oleic acid. The suspension may be applied to the entire device, or to a portion thereof, by any method known in the art. In certain applications, the solid particles may be left in the coating; alternatively, they may be removed by sublimation to for the pores or spaces.

A medical device may have a passageway through which body fluids may pass and may further comprise an enclosed internal space for containing one or more compounds therein. The passageway may comprise one or more pores that allow delivery or diffusion of compound(s) from the enclosed internal space into the lumen of the passageway. The device may be positioned in the body so as to deliver the compounds over a period of time to the appropriate location at the desired level or volume, dependent on the size of the pores and the characteristics of the composition.

Further description and examples of reservoirs, pores, divits, holes, micropores, or channels on the surface of or within medical devices may be found in the following: U.S. Pat. No. 6,652,581; U.S. Patent Application Nos. 2001/0029660; 2004/0215169; and 2006/0088567; PCT Application Nos. WO 01/87372; WO 02/32347; WO 03/015664; WO 2004/026174; WO 2004/026182; WO 2004/026357; WO 2004/043509; WO 2004/043511; WO 2004/087011; WO 2004/087214; WO 2004/087251; WO 2004/108186; WO 2004/110302; WO 2005/046521; WO 2005/079387; WO 2005/102222; WO 2005/120397; WO 2006/012034; WO 2006/012060; WO 2006/098889; WO 2006/099381; WO 2006/105126; and WO 2006/105256

Differential coating of a stent may be accomplished by coating each of two stent members with a different coating composition, wherein one may contain one compound (e.g., paclitaxel) and the second another compound (e.g., dipyridamole). In a particular aspect of this embodiment, the two stent member have diameters such that one stent will fit inside of the other. One or both of the stent members may be separately coated, after which one is placed inside of the other to form the final stent. This provides a stent with one composition on the outside surface and another composition on the inside surface. Alternatively, the final stent may have a coating on only the outside surface or only the inside surface. Further description of this aspect may be found in U.S. Patent Application No. 2005/0192662.

Within certain embodiments of this disclosure, the carrier can also comprise radio-opaque, echogenic materials and magnetic resonance imaging (MRI) responsive materials (i.e., MRI contrast agents) to aid in visualization of the device under ultrasound, fluoroscopy and/or MRI. For example, a device may be made with or coated with a composition which is echogenic or radiopaque (e.g., made with echogenic or radiopaque with materials such as powdered tantalum, tungsten, barium carbonate, bismuth oxide, barium sulfate, metrazimide, iopamidol, iohexyl, iopromide, iobitridol, iomeprol, iopentol, ioversol, ioxilan, iodixanol, iotrolan, acetrizoic acid derivatives, diatrizoic acid derivatives, iothalamic acid derivatives, ioxithalamic acid derivatives, metrizoic acid derivatives, iodamide, lypophylic agents, iodipamide and ioglycamic acid or, by the addition of microspheres or bubbles which present an acoustic interface). Visualization of a device by ultrasonic imaging may be achieved using an echogenic coating. Echogenic coatings are described in, e.g., U.S. Pat. Nos. 6,106,473 and 6,610,016. For visualization under MRI, contrast agents (e.g., gadolinium (III) chelates or iron oxide compounds) may be incorporated into or onto the device, such as, for example, as a component in a coating or within the void volume of the device (e.g., within a lumen, reservoir, or within the structural material used to form the device). In some embodiments, a medical device may include radio-opaque or MRI visible markers (e.g., bands) that may be used to orient and guide the device during the implantation procedure.

In another embodiment, these agents can be contained within the same coating layer as the compound or they may be contained in a coating layer (as described above) that is either applied before or after the layer containing the combination of compounds.

Medical implants may, alternatively, or in addition, be visualized under visible light, using fluorescence, or by other spectroscopic means. Visualization agents that can be included for this purpose include dyes, pigments, and other colored agents. In one aspect, the medical implant may further include a colorant to improve visualization of the implant in vivo and/or ex vivo. Frequently, implants can be difficult to visualize upon insertion, especially at the margins of implant. A coloring agent can be incorporated into a medical implant to reduce or eliminate the incidence or severity of this problem. The coloring agent provides a unique color, increased contrast, or unique fluorescence characteristics to the device. In one aspect, a solid implant is provided that includes a colorant such that it is readily visible (under visible light or using a fluorescence technique) and easily differentiated from its implant site. In another aspect, a colorant can be included in a liquid or semi-solid composition. For example, a single component of a two component mixture may be colored, such that when combined ex-vivo or in-vivo, the mixture is sufficiently colored.

The coloring agent may be, for example, an endogenous compound (e.g., an amino acid or vitamin) or a nutrient or food material and may be a hydrophobic or a hydrophilic compound. Preferably, the colorant has a very low or no toxicity at the concentration used. Also preferred are colorants that are safe and normally enter the body through absorption such as β-carotene. Representative examples of colored nutrients (under visible light) include fat soluble vitamins such as Vitamin A (yellow); water soluble vitamins such as Vitamin B12 (pink-red) and folic acid (yellow-orange); carotenoids such as β-carotene (yellow-purple) and lycopene (red). Other examples of coloring agents include natural product (berry and fruit) extracts such as anthocyanin (purple) and saffron extract (dark red). The coloring agent may be a fluorescent or phosphorescent compound such as α-tocopherolquinol (a Vitamin E derivative) or L-tryptophan. Derivatives, analogues, and isomers of any of the above colored compound also may be used. The method for incorporating a colorant into an implant or therapeutic composition may be varied depending on the properties of and the desired location for the colorant. For example, a hydrophobic colorant may be selected for hydrophobic matrices. The colorant may be incorporated into a carrier matrix, such as micelles. Further, the pH of the environment may be controlled to further control the color and intensity.

In one aspect, the composition of the present disclosure include one or more coloring agents, also referred to as dyestuffs, which will be present in an effective amount to impart observable coloration to the composition, e.g., the gel. Examples of coloring agents include dyes suitable for food such as those known as F.D. & C. dyes and natural coloring agents such as grape skin extract, beet red powder, beta carotene, annato, carmine, turmeric, paprika, and so forth. Derivatives, analogues, and isomers of any of the above colored compound also may be used. The method for incorporating a colorant into an implant or therapeutic composition may be varied depending on the properties of and the desired location for the colorant. For example, a hydrophobic colorant may be selected for hydrophobic matrices. The colorant may be incorporated into a carrier matrix, such as micelles. Further, the pH of the environment may be controlled to further control the color and intensity.

In one aspect, the compositions of the present disclosure include one or more preservatives or bacteriostatic agents, present in an effective amount to preserve the composition and/or inhibit bacterial growth in the composition, for example, bismuth tribromophenate, methyl hydroxybenzoate, bacitracin, ethyl hydroxybenzoate, propyl hydroxybenzoate, erythromycin, 5-fluorouracil, methotrexate, doxorubicin, mitoxantrone, rifamycin, chlorocresol, benzalkonium chlorides, and the like. Examples of the preservative include paraoxybenzoic acid esters, chlorobutanol, benzylalcohol, phenethyl alcohol, dehydroacetic acid, sorbic acid, etc. In one aspect, the compositions of the present disclosure include one or more bactericidal (also known as bactericidal) agents.

Within certain embodiments of this disclosure, the described compositions may also comprise additional ingredients such as surfactants (e.g., PLURONICS, such as F-127, L-122, L-101, L-92, L-81, and L-61), anti-inflammatory agents (e.g., dexamethasone or asprin), anti-thrombotic agents (e.g., heparin, high activity heparin, heparin quaternary amine complexes (e.g., heparin benzalkonium chloride complex)), anti-infective agents (e.g., 5-fluorouracil, triclosan, rifamycin, and silver compounds), preservatives, anti-oxidants and/or anti-platelet agents.

In one aspect, the compositions of the present disclosure include one or more antioxidants, present in an effective amount. Examples of the antioxidant include sulfites, alpha-tocopherol and ascorbic acid.

The total amount of each compound made available on, in or near the device may be in an amount ranging from about 0.01 μg (micrograms) to about 2500 mg (milligrams). Generally, the compounds may be in the amount ranging from 0.01 μg to about 10 μg; or from 10 μg to about 1 mg; or from 1 mg to about 10 mg; or from 10 mg to about 100 mg; or from 100 mg to about 500 mg; or from 500 mg to about 2500 mg.

The total amount of each compound on, in or near the device may be in an amount ranging from less than 0.01 μg to about 2500 μg per mm² of device surface area. Generally, a compound may be present in an amount ranging from less than 0.01 μg; or from 0.01 μg to about 1.0 μg; or from 0.01 μg to about 10 μg; or from about 0.5 μg to about 5 μg; or from about 0.05 μg to 50 μg; or from 10 μg to about 250 μg; or from 250 μg to about 2500 μg (per mm² of device surface area).

In certain embodiments, paclitaxel is present in an amount ranging from about 0.01 to about 1.0 μg/mm² and dipyridamole is present in an amount ranging from about 0.05 to about 50 μg/mm².

In other embodiments, paclitaxel is present in an amount ranging from about 0.1 to about 0.6 μg/mm² and dipyridamole is present in an amount ranging from about 0.5 to about 5 μg/mm².

In one embodiment, the weight ratio of dipyridamole to paclitaxel may exceed about 0.06 to about 1.0 to provide a superior biological effect. The weight ratio of dipyridamole to paclitaxel may be adjusted to exceed about 0.06; or about 0.08; or about 0.10; or about 0.20; or about 0.30 or about 0.40; or about 0.50; or about 0.60; or about 0.70; or about 0.80; or about 0.90; or about 1.0; or about 1.1; or about 1.2; or about 1.3; or about 1.4; or about 1.5; or about 1.6.

In certain embodiments, the therapeutic composition should be biocompatible, and release one or more compounds over a period of several hours, days, or, months. As described above, “release of an agent” refers to any statistically significant presence of the agent, or a subcomponent thereof, which has disassociated from the compositions and/or remains active on the surface of (or within) the composition. The compositions of the present disclosure may release the compounds at one or more phases, the one or more phases having similar or different performance (e.g., release) profiles. The compounds may be made available to the tissue at amounts which may be sustainable, intermittent, or continuous; in one or more phases; and/or rates of delivery; effective to reduce or inhibit any one or more components of fibrosis (or scarring), including: formation of new blood vessels (angiogenesis), platelet adherence, infiltration of inflammatory cells (such as white blood cells), activation of white blood cells and other inflammatory cells and cytokines, fibrin deposition, migration and proliferation of connective tissue cells (such as fibroblasts or smooth muscle cells), deposition of extracellular matrix (ECM), and remodeling (maturation and organization of the fibrous tissue).

Thus, release rate may be programmed to impact fibrosis (or scarring) by releasing a compound at a time such that at least one of the components of fibrosis is inhibited or reduced. Moreover, the predetermined release rate may reduce agent loading and/or concentration as well as potentially providing minimal drug washout and thus, increases efficiency of drug effect. Any one of the compounds may perform one or more functions, including inhibiting the formation of new blood vessels (angiogenesis), inhibiting platelet adherence, preventing or reducing the infiltration of inflammatory cells (such as white blood cells), inhibiting the function or activity of inflammatory cells, reducing the production of (or the effects of) proinflammatory cytokines, reducing fibrin deposition, inhibiting the migration and proliferation of connective tissue cells (such as fibroblasts or smooth muscle cells), inhibiting the deposition of extracellular matrix (ECM), and inhibiting remodeling (maturation and organization of the fibrous tissue). In one embodiment, the rate of release may provide a sustainable level of the compound to the susceptible tissue site. In another embodiment, the rate of release is substantially constant. The rate may decrease and/or increase over time, and it may optionally include a substantially non-release period. The release rate may comprise a plurality of rates. The release rate of one compound (e.g. paclitaxel or an analogue or derivative thereof) may be different from the release rate of the other therapeutic compound (e.g. dipyridamole or an analogue or derivative thereof). The ratio of the amount of one compound (e.g. paclitaxel or an analogue or derivative thereof) relative to the other therapeutic compound (e.g. dipyridamole or an analogue or derivative thereof) may be the same throughout or differ over the course of its administration. In an embodiment, the plurality of release rates may be substantially constant, decreasing, increasing, or substantially non-releasing.

In one embodiment, the compound(s) is made available to the susceptible tissue site in a programmed, sustained, and/or controlled manner which results in increased efficiency and/or efficacy. Further, the release rates may vary during either or both of the initial and subsequent release phases. There may also be additional phase(s) for release of the same substance(s) and/or different substance(s).

The compound that is on, in or near the device may be released from the composition in a time period that may be measured from the time of implantation, which ranges from about less than 1 day to about 180 days. Generally, the release time may be from about less than 1 day to about 7 days. However, release times may range from less than 1 day to about 7 days; or to about 14 days; or to about 28 days; or to about 56 days; or to about 90 days; or to about 180 days.

The amount of compound released from the composition as a function of time may be determined based on the in vitro release characteristics of the agent from the composition. The in vitro release rate may be determined by placing the compound within the composition or device in an appropriate buffer solution at 37° C. Samples of the buffer solution are then periodically removed for analysis by HPLC, and the buffer is replaced periodically.

Based on the in vitro release rates, the release of compound(s) per day may range from an amount ranging from about 0 μg (micrograms) to about 2500 mg (milligrams). Generally, the compound(s) that may be released in a day may be in the amount ranging from 0 μg to about 10 μg; or from 10 μg to about 1 mg; or from 1 mg to about 10 mg; or from 10 mg to about 100 mg; or from 100 mg to about 500 mg; or from 500 mg to about 2500 mg.

Further, therapeutic compositions and devices of the present disclosure should preferably have a stable shelf-life for several months and capable of being produced and maintained under sterile conditions. Many pharmaceuticals are manufactured to be sterile and this criterion is defined by the USP XXII <1211>. The term “USP” refers to U.S. Pharmacopeia (see www.usp.org, Rockville, Md.). Sterilization may be accomplished by a number of means accepted in the industry and listed in the USP XXII <1211>, including gas sterilization, ionizing radiation or, when appropriate, filtration. Sterilization may be maintained by what is termed aseptic processing, defined also in USP XXII <1211>.

Acceptable gases used for gas sterilization include ethylene oxide. Acceptable radiation types used for ionizing radiation methods include gamma, for instance from a cobalt 60 source and electron beam. A typical dose of gamma radiation is 2.5 MRad. Filtration may be accomplished using a filter with suitable pore size, for example 0.22 μm and of a suitable material, for instance polytetrafluoroethylene (e.g., TEFLON from E.I. DuPont De Nemours and Company, Wilmington, Del.).

In another aspect, the compositions and devices of the present disclosure are contained in a container that allows them to be used for their intended purpose, i.e., as a pharmaceutical composition. Properties of the container that are important are a volume of empty space to allow for the addition of a constitution medium, such as water or other aqueous medium, e.g., saline, acceptable light transmission characteristics in order to prevent light energy from damaging the composition in the container (refer to USP XXII <661>), an acceptable limit of extractables within the container material (refer to USP XXII), an acceptable barrier capacity for moisture (refer to USP XXII <671>) or oxygen. In the case of oxygen penetration, this may be controlled by including in the container, a positive pressure of an inert gas, such as high purity nitrogen, or a noble gas, such as argon.

Typical materials used to make containers for pharmaceuticals include USP Type I through III and Type NP glass (refer to USP XXII <661>), polyethylene, TEFLON, silicone, and gray-butyl rubber.

In one embodiment, the product containers can be thermoformed plastics. In another embodiment, a secondary package can be used for the product. In another embodiment, product can be in a sterile container that is placed in a box that is labeled to describe the contents of the box.

D. Methods of Associating Compounds with a Device
1) Devices that Include or Release Compounds

Devices may be adapted to release paclitaxel and dipyridamole (and/or analogues or derivatives thereof) by methods including: (a) directly affixing to the device a desired compound or composition containing the compound (e.g., by either spraying or electrospraying the device with a compound and/or carrier (polymeric or non-polymeric)-compound composition to create a film and/or coating on all, or parts of the internal and/or external surface of the device; by dipping the device into a compound and/or carrier (polymeric or non-polymeric)-compound solution to coat all or parts of the device; or by other covalent or noncovalent attachment of the compound to the device surface); (b) by coating the device with a substance such as a hydrogel which either contains or which will in turn absorb the desired compounds or composition; (c) by interweaving a “thread” composed of, or coated with, the compound into the device; (d) by covering all, or portions of the device with a sleeve, cover, electrospun fabric or mesh containing the compounds (i.e., a covering comprised of a compound or polymerized compositions containing one or both compounds); (e) constructing all, or parts of the device itself with the desired compounds or composition containing the compounds or polymerized compositions of compounds); (f) otherwise impregnating the device with the compounds or a composition containing the compounds; (g) constructing all, or parts of the device or implant itself from a degradable or non-degradable polymer that releases one or more compounds; (i) utilizing specialized multi-drug releasing medical device systems (for example, U.S. Pat. Nos. 6,527,799; 6,293,967; 6,290,673; 6,241,762, U.S. Application Publication Nos. 2003/0199970A1 and 2003/0167085A1, and PCT Publication WO 03/015664) to deliver compounds alone or in combination.

2) Coating of Devices with Compounds

As described above, a range of polymeric and non-polymeric materials can be used to incorporate the compounds onto or into a device. The compound-containing composition can be incorporated into or onto the device in a variety of ways. Coating of the device with the compound-containing composition or with the compounds only is one process that can be used. The compounds, with or without being formulated into a composition, may be coated onto the entire device or a portion of the device using a method that is appropriate for the particular type of device, including, but not limited to, dipping, spraying, rolling, brushing, painting, electrostatic plating or spinning, vapor deposition, air spraying, including atomized spray coating, and spray coating with an ultrasonic nozzle.

a) Dip Coating

Dip coating is one coating process that can be used. When possible, the dip coating procedure is performed using evaporative solvents of high vapor pressure to produce the desired viscosity and quickly establish coating layer thicknesses. In one embodiment, the compounds are dissolved in a solvent and then coated onto the device.

b) Spray Coating

Spray coating is another coating process that can be used. In the spray coating process, a solution or suspension of the compounds, with or without a polymeric or non-polymeric carrier, is nebulized or atomized and directed to the device to be coated by a stream of gas, such as nitrogen. One can use spray devices such as an air-brush (for example models 2020, 360, 175, 100, 200, 150, 350, 250, 400, 3000, 4000, 5000, 6000 from Badger Air-brush Company, Franklin Park, Ill.), spray painting equipment, TLC reagent sprayers (for example Part #14545 and 14654, Alltech Associates, Inc. Deerfield, Ill.), and ultrasonic spray devices (for example those available from Sono-Tek, Milton, N.Y.). One can also use powder sprayers and electrostatic sprayers. Further, during spray coating of a device, the device is typically rotated. In a particular aspect, for example, a rotating radially expanded stent is sprayed using an air brush. When possible, solvent materials of relatively high vapor pressure are used to produce the desired viscosity and quickly establish coating layer thicknesses. The coating process enables the material to adhere and conform to the entire surface of the open stent, or other device, such that the open lattice nature of the structure of the stent is preserved in the coated device. During spray coating, the speed of rotation and the flow rate of the nozzle may be adjusted as desired to modify the nature of the layering. In one representative aspect, when rotating a stent to be spray coated, the stent may be held by clips in a horizontal orientation in its expanded state for rotation. Further, for example, the speed of rotation may be 30-50 rpm and the flow rate 4-10 ml of coating composition per minute. The viscosity of the composition may also be adjusted, which will affect the selection of the other parameters. Several layers may be applied to a single device, with the initial layers being referred to as tie layers. The additional layers external to the tie layers may have a different composition, particularly with respect to content of compound, as well as polymer components and cross-linking agents, when present.

In another embodiment, a device, such as a stent, may be electrostatically spray coated. In a particular example, an electrically charged conductive core wire is arranged axially through the center of a stent. The wall of the stent is either grounded or electrically charged. Upon application of an electrical charge to the core wire and exposure of the stent and the core wire to an electrically charged coating formulation, delivered by an air brush, for example, the coating formulation is deposited on the surfaces of the stent. The charge on the stent and the core wire may be alternated, as desired, depending on the charge characteristics of the coating formulation.

Methods for spray deposition of materials onto small targets may include use of a fine-bore diameter spray nozzle body to pressurize the coating material within the nozzle body and dampening vibration of the nozzle body during operation. Methods may further include achieving a finer atomized spray droplet size by pre-filming the coating material onto a flat face before entraining the coating material with the atomizing fluid. Further description of these methods may be found in U.S. Patent Application No. 2005/0202156. A system and a method for differentially coating a medical device having an interior is described in U.S. Patent Application No. 2005/0238829.

Coating compositions may be formulated according to the particular procedure used to apply the coating. For example, the composition used for spray coating may differ from that used for dip coating.

In one embodiment, the compound is dissolved in a solvent and then sprayed onto the device.

c) Roll Coating

Roll coating is another coating process that can be used. According to this process, devices are placed into holders that rotate. The holders are placed on a conveyer belt, which moves each device/holder toward the coating region of the apparatus. Upon reaching the coating region, the holders rotate, thus exposing multiple surfaces of the device to a spray. An example of this process is described in U.S. Patent Application No. 2005/0158450.

### E. Medical Implants and Methods of Using Medical Implants

There are numerous medical devices where the occurrence of a fibrotic reaction will adversely affect the functioning of the device or the biological problem for which the device was implanted or used. Representative examples of implants or devices that can be associated with or otherwise constructed to contain and/or release the compounds provided herein include intravascular stents (e.g., coronary and peripheral vascular stents), non-vascular stents (e.g., tracheal stents, bronchial stents, GI stents, and the like), devices, anastomotic connector devices, vascular grafts, hemodialysis access devices, soft tissue implants (such as breast implants, facial implants, tissue fillers, aesthetic implants and the like), implantable electrodes (cardiac pacemakers, neurostimulation devices), implantable sensors, drug delivery pumps, anti-adhesion solution and barriers, and shunts.

The association of a combination of paclitaxel and dipyridamole (or analogues or derivatives thereof) onto, or incorporation of a combination of paclitaxel and dipyridamole (or analogues or derivatives thereof) into medical devices provides a solution to the clinical problems that can be encountered with these devices. Alternatively, or additional, compositions that comprise a combination of paclitaxel and dipyridamole (or analogues or derivatives thereof) can be infiltrated into the space or onto tissue surrounding the area where medical devices are implanted either before, during or after implantation of the devices.

Described below are examples of medical devices whose functioning can be improved by the use of a combination of compounds as well as methods for incorporating compounds into or onto these devices and methods for using such devices.

Intravascular Devices

The present disclosure provides for the combination of compounds and an intravascular device.

“Intravascular devices” refers to devices that are implanted at least partially within the vasculature (e.g., blood vessels). Examples of intravascular devices that can be used to deliver the combination of compounds to the desired location include, e.g., catheters, balloon catheters, balloons, stents, covered stents, anastomotic connectors, vascular grafts, hemodialysis access devices, guidewires, and the like.

Intravascular Stent

In one aspect, the present disclosure provides for the combination of paclitaxel and dipyridamole (or analogues or derivatives thereof) or a composition comprising a combination of paclitaxel and dipyridamole (or analogues or derivatives thereof) and an intravascular stent.

“Stent” refers to devices comprising a cylindrical tube (composed of a metal, textile, non-degradable or degradable polymer, and/or other suitable material (such as biological tissue) which maintains the flow of blood from one portion of a blood vessel to another. In one aspect, a stent is an endovascular scaffolding which maintains the lumen of a body passageway (e.g., an artery) and allows bloodflow. Representative examples of stents that can benefit from being coated with or having associated with, the described compounds include vascular stents, such as coronary stents, peripheral stents, and covered stents.

Stents that can be used in the present disclosure include metallic stents, polymeric stents, biodegradable stents and covered stents. Stents may be self-expandable or balloon-expandable, composed of a variety of metal compounds and/or polymeric materials, fabricated in innumerable designs, used in coronary or peripheral vessels, composed of degradable and/or nondegradable components, fully or partially covered with vascular graft materials (so called “covered stents”) or “sleeves”, and can be bare metal or drug-eluting.

Stents may be comprise a metal or metal alloy such as stainless steel, spring tempered stainless steel, stainless steel alloys, gold, platinum, super elastic alloys, cobalt-chromium alloys and other cobalt-containing alloys (including ELGILOY (Combined Metals of Chicago, Grove Village, Ill.), PHYNOX (Alloy Wire International, United Kingdom) and CONICHROME (Carpenter Technology Corporation, Wyomissing, Pa.)), titanium-containing alloys, platinum-tungsten alloys, nickel-containing alloys, nickel-titanium alloys (including nitinol), malleable metals (including tantalum); a composite material or a clad composite material and/or other functionally equivalent materials; and/or a polymeric (non-biodegradable or biodegradable) material. Representative examples of polymers that may be included in the stent construction include polyethylene, polypropylene, polyurethanes, polyesters, such as polyethylene terephthalate (e.g., DACRON or MYLAR (E. I. DuPont De Nemours and Company, Wilmington, Del.)), polyamides, polyaramids (e.g., KEVLAR from E.I. DuPont De Nemours and Company), polyfluorocarbons such as poly(tetrafluoroethylene with and without copolymerized hexafluoropropylene) (available, e.g., under the trade name TEFLON (E. I. DuPont De Nemours and Company), silk, as well as the mixtures, blends and copolymers of these polymers. Stents also may be made with engineering plastics, such as thermotropic liquid crystal polymers (LCP), such as those formed from p,p′-dihydroxy-polynuclear-aromatics or dicarboxy-polynuclear-aromatics.

Further types of stents that can be used with the described compounds are described, e.g., in PCT Publication No. WO 01/01957 and WO0003661 and U.S. Pat. Nos. 6,736,842; 6,607,553; 6,620,201; 6,165,210; 6,099,561; 6,071,305; 6,063,101; 5,997,468; 5,980,551; 5,980,566; 5,972,027; 5,968,092; 5,951,586; 5,893,840; 5,891,108; 5,851,231; 5,843,172; 5,837,008; 5,766,237; 5,769,883; 5,735,811; 5,700,286; 5,683,448; 5,679,400; 5,665,115; 5,649,977; 5,637,113; 5,591,227; 5,551,954; 5,545,208; 5,500,013; 5,464,450; 5,419,760; 5,411,550; 5,342,348; 5,286,254; and 5,163,952. Removable drug-eluting stents are described, e.g., in U.S. Pat. Nos. 6,981,987; 6,494,908 and 5,882,335 and in Lambert, T. (1993) J. Am. Coll. Cardiol.: 21: 483A. Moreover, the stent may be adapted to release the desired compound at only the distal ends, or along the entire body of the stent. For example, Advanced Cardiovascular Systems (Santa Clara, Calif.) is developing an eluting sheath fabricated from a mesh that may be attached to at least a portion of an outside surface area of the stent structure as described in U.S. Pat. No. 7,105,018. In another example, Advanced Cardiovascular Systems describes a polymeric material, such as polyurethane or ePTFE which is used to cover or partially cover an intravascular stent which may be provided with holes to permit endothelialization and/or drug loading. See, for example, U.S. Pat. No. 7,118,592.

Balloon over stent devices, such as are described in Wilensky, R. L. (1993) J. Am. Coll. Cardiol.: 21: 185A, also are suitable for local delivery of compounds to a treatment site.

Stents may be coated with a polymeric drug delivery system to deliver the combination of paclitaxel and dipyridamole (or analogues or derivatives thereof). In addition to there being a variety of polymeric formulations to deliver the compound from a stent, the stent may also be coated in a variety of ways, for example, by spraying, dipping, deposition or painting. For example, Lombard Medical (Oxford, UK) manufactures a family of drug delivery polymers with programmable elution profile technology. This coating technology allows for several drugs to be released from a coating at different times and in different quantities from a drug-eluting stent. Another example of a polymeric stent coating is the desaminotyrosine polyarylate biodegradable coating made by TyRx Pharma (New Brunswick, N.J.). Another example of a polymeric stent coating is a biomimetic (triblock copolymer) coating being made by Allvivo (Lake Forest, Calif.) that incorporates a drug which is tethered to the stent surface using polyethylene oxide. See, for example, US Patent Application No. 2005/0106208 and PCT Publication Nos. WO05118020; WO05042025; and WO04037310. Another example of a polymeric stent coating is made by TissueGen (Dallas, Tex.) which is based on biodegradable, drug-releasing polymer fiber scaffolds. Another example of a polymeric stent coating is the biodegradable tyrosine-derived polycarbonates that provide radiography/fluoroscopy visibility for accuracy in placement and continued monitoring after implantation, which is manufactured by New Jersey Center for Biomaterials (Piscataway, N.J.). Another example of a polymeric stent coating is a polylactic acid bioerodible polymer manufactured by Biosensors International (Singapore) that biodegrades to carbon dioxide and water. Biosensors International also manufactures the BIO-MATRIX stent, MATRIX stent, S-STENT and the CHALLENGE drug-eluting stent. The CHAMPION stent (Guidant, St. Paul, Minn.) has also been coated with Biosensor's coating technology and similarly Terumo Corp. (Japan) is also utilizing Biosensors technology platform. Another example of a polymeric stent coating is a thin film coating technology combined with a microporous biocompatible CHRONOFLEX polycarbonate/polyurethane technology developed by Cornova [joint venture between Implant Sciences (Wakefield, Mass.) and Cardiotech (Woburn, Mass.)]. See, for example, PCT Publication No. WO02072167. Another example of a polymeric stent coating is the biodegradable programmable amino acid polymer coating technology from Medivas (San Diego, Calif.). See, for example, US Patent Application Nos. 2006/0188486; 2006/0013855; 2004/0170685 and PCT Publication Nos. WO06088647 and WO04075781. Another example of a polymeric stent coating is that developed by Abbott Laboratories (Abbott Park, Ill.) under the name of TRIMAXX stent which is coated with phosphorylcholine that elutes drug over a 30 day period. See, for example, U.S. Pat. No. 6,890,546 and US Patent Application No. 2006/0198867 and PCT Publication Nos. WO06102359 and WO06050170. Another example of a polymeric stent coating is a non-biodegradable poly(styrene-b-isobutylene-b-styrene) known as TRANSLUTE-polymer that provides an initial burst phase during the initial 48 hours followed by a slow release over the next 10 days with no further release after 30 days. This is the coating that Boston Scientific (Natick, Mass.) uses on its TAXUS EXPRESS and LIBERTE drug-eluting stents. See, for example, U.S. Pat. Nos. 7,096,554; 6,984,411; 6,918,869; 6,908,622; 6,620,194; 6,358,556; 6,306,166; 6,284,305; 6,042,875 and US Patent Application Nos. 2006/0089705 and 2005/0106210. Another example of a stent coating is a combination of three layers of polymers know as the BRAVO Drug Delivery Polymer Matrix which was developed by Surmodics (Eden Prairie, Minn.) which is used on the CYPHER drug-eluting stent from Cordis (subsidiary of J&J; Miami Lakes, Fla.) as well as the ETHOS Drug-Eluting Coronary Stent System from X-Cell Medical (Princeton, N.J.). These three layers of BRAVO are composed of a primer coating of Parylene C onto which is sprayed a solution of two biodegradable polymers, polyethylene-co-vinyl acetate (PEVA) and poly n-butyl methacrylate (PBMA) that contains the drug. The top layer is a drug-free coating of a solution of both PEVA and PBMA that serves to control drug release and prevent a burst effect. The drug is released during the first week after implantation and 85% of the drug is released over 30 days. Surmodics also develops other coatings such as the ENCORE Drug Delivery Polymer Matrix, which is a proprietary blend of PBMA and poly-butadiene (PBD). These blends may be varied by ratio in the coating to adjust drug delivery rates and mechanical properties. Surmodics also makes the SYNBIOSYS Biodegradable Drug Delivery System which is a proprietary family of multiblock copolymers constructed from base units of glycolide, lactide, e-caprolactone and polyethylene glycol which are biodegradable. The SYNBIOSIS technology is used on the XTRM-FIT Coronary Stent for the Melatonin-Eluting Stent System developed by Millimed (Sweden) and Blue Medical (Netherlands). The EUREKA Biodegradable Drug Delivery Matrix is Surmodics nano-engineered polysaccharides. The CAMEO Biodegradable Drug Delivery Matrix is Surmodics proprietary blend of poly(ester-amide) homologs based on leucine or phenylalanine. Surmodics also makes the POLYACTIVE Biodegradable Polymeric Drug Delivery System which is composed of a family of co-polymers offering a range of release rates simply by varying the monomer ratio in the polymer or the size of hydrophilic monomer component. Surmodics hydrophilic technology has been licensed to Devax (Irvine, Calif.) to provide the lubricious coating on its AXXESS Biolimus A-9 Eluting Bifurcation Stent Delivery System. Coatings made by Surmodics are described, for example, in U.S. Pat. No. 6,254,634 and PCT Publication Nos. WO06107336; WO06002112; WO05099787; WO05097222; and WO9964086. Another example of polymeric stent coatings are those described by Johnson and Johnson and its subsidiaries Ethicon (Sommerville, N.J.) and Cordis Corporation (Miami Lakes, Fla.). These stent coatings include, for example, (a) a biocompatible film of polyfluoro copolymer (see e.g., U.S. Pat. No. 6,746,773), (b) coatings that are saturated and then spun off repetitively to form a dry, non-sticky conforming coating (see e.g., U.S. Pat. No. 6,723,373), (c) thin film polymers using a supercritical carbon dioxide process (see e.g., U.S. Pat. No. 6,627,246), (d) film of heptafluorobutylmethacrylate that is applied to a stent surface by radiofrequency plasma deposition and subsequently treated with a biologically active agent (see e.g., U.S. Pat. No. 5,336,518); (e) an aqueous latex polymeric emulsion that is applied to a stent via dipping and drying the aqueous latex polymeric emulsion to form the coating (see e.g., U.S. Pat. No. 6,919,100); (f) a stent with micropores or reservoirs in the stent body in which compounds is mixed or bound to a polymer coating directly on the stent (see e.g., U.S. Pat. Nos. 6,585,764 and 6,273,913); (g) a coating of endothelial cell specific adhesion peptides to promote endothelial cell attachment, which is activated with plasma glow discharge and a plurality of polymeric layers (see e.g., U.S. Pat. No. 6,140,127); (h) heparin coating composed of multiple layers (see e.g., U.S. Pat. No. 5,876,433); (i) coating that has bioactive properties and contains an embedded radioisotope that makes the coating material radioactive (See e.g., U.S. Pat. No. 5,722,984). These stent coatings as well as other polymeric and non-polymeric coatings manufactured by Johnson and Johnson and its subsidiaries are described in, for example, U.S. Pat. Nos. 7,041,088; 7,030,127; 6,838,491; 6,776,796; 6,623,823; 6,537,312; 6,153,252; 5,891,108; and 5,163,958. Another example of a polymeric stent coating is the nanospun coatings being manufactured to elute nitric oxide by Millimed (Sweden). Another example of a polymeric stent coating is a bioabsorbable polymer that is mixed and bound to the stent which is absorbed after three weeks. This polymeric coating is being developed by Blue Medical (Netherlands) in association with Creganna Medical Devices (Ireland) and is described, for example, in PCT Publication No. WO05016400. Another example of a polymeric stent coating is the microporous and ultra-thin ADVANTA PTFE film that may be used to encapsulate stent tines. Atrium Medical (Hudson, N.H.) utilizes this coating technology for their ADVANTA V12 Covered Stent and ICAST Covered Stent. See, for example, US Patent Application Nos. 2006/0088596; 2006/0067977 and 2005/0158361 and PCT Publication Nos. WO06036967 and WO06036970. Another example of a polymeric stent coating is that used on the APOLLO Drug-Eluting Stent made by Intek Technology (Baar, Switzerland). This stent coating is an elastomeric, biostable, hemocompatible controlled release system which covers the stent struts all the way around having a thicker coating on the exterior side of the stent compared to the inner side. Another example of a polymeric stent coating are coatings that are sprayed on, for example the ELECTRONANOSPRAY technology from Nanocopoeia (St. Paul, Minn.) and CRITICOAT from Micell Technologies. This technology allows drug to be sprayed onto the stent in the form of nanoparticles. In the case of CRITICOAT, the drug morphology and stability is maintained as there is no need for a liquid solvent as is necessary for conventional methods of coating medical devices and formulating drugs. Another example of a polymeric stent coating is the VECTOR Coating of the VITASTENT made by Aachen Resonance (Germany), which is a stable thin functionalized polymer layer formed by monomers in the gas phase with a bioactive layer containing active agent. The VECTOR Coating reduces platelet activation and has improved biocompatibility and is described, for example, in PCT Publication No. WO03077967. Another example of a polymeric stent is a layer composed of poly(para-xylylene) which is coated onto a stent by chemical vapor deposition with a second polymer layer of poly(vinyl alcohol)-graft-poly(lactide-co-glycolide) using the spray coating technique. This polymeric coating may be applied onto many types of stents, such as the JOSTENT made by Jomed (Sweden), as described in, for example, Westedt et al., J. Controlled Rd. (2006), 111(1-2): 235-246. Another example of a polymeric stent coating is a heparin diffusion barrier fixed to a polymeric coating to control elution rate of a compound, which is being developed by Cordis (subsidiary of J&J; Miami Lakes, Fla.) and described in, for example, US Patent Application No. 2005/0004663. Ethicon Another example of a polymeric stent coating is PICO ELITE Paclitaxel-Eluting Stent made by AMG GmbH (Germany), which is based on the ARTHROS PICO cobalt chromium stent, which is surface coated with a biostable polymer containing paclitaxel. Another example of a polymeric stent coating is that being used on the TAXOCHROME Drug-Eluting stent developed by DISA Vascular (South Africa), which is a bio-absorbable polymer that allows for both early-stage and late-stage elution through gradual but complete polymer erosion within two months. Another example of a polymeric stent coating is that being used for the INFINNIUM Paclitaxel-Eluting Stent which is made by Sahajanand Medical Technologies PVT LTD. (India), which is a biodegradable polymer-based system. The coating for INFINNIUM is based on multiple layers of successive biodegradable polymer formulations based on poly-D,L-lactide-co-glycolide, poly L lactide-co-caprolactone, poly L-lactide and poly vinyl pyrrolidone. See, for example, Kothwala et al., Trends Biomater. Artif. Organs, (2006) 19(2): 88-92. Another example of a polymeric stent coating is the UNICOAT technology used on Pimecrolimus-Eluting DURAFLEX stent made by Avantec Vascular Corp. (Sunnyvale, Calif.). UNICOAT is based on a proprietary biocompatible and non-resorbable polymer. Another example of a polymeric stent coating is a film composed of poly(vinyl alcohol)-graft-poly(lactide-co-glycolide) as described, for example, in Westedt et al., J. Controlled Rel., (2006) 111(1-2): 235-246.

Stents may be combined with a drug delivery system to deliver the compounds. For example, MIV Therapeutics, Inc. (Vancouver, BC, Canada) makes biocompatible coatings and advanced drug delivery systems for cardiovascular stents and other implantable medical devices based on hydroxyapatite (HAp), which is naturally occurring polymer found in bone and tooth enamel. These HAp coatings are a deposition of dense ultra-thin Hap as well as microporous thicker HAp films designated to carry drugs for slow release following implantation. The microporous films are designed to remain highly biocompatible even after all drug is eluted from the coating and is intended to inhibit the inflammatory response elicited by bare metal stents. See for example, US Patent Application No. 2006/0134211 and PCT Publication Nos. WO06063430 and WO06024125. Another example of a stent coating is RAINBOW COATING developed by Translumina (Hechingen, Germany), which is a passive diamond-like carbon nanolayer coating that is applied by plasma-assisted chemical vapor deposition for coronary and peripheral stents to increase biocompatibility. This non-polymer carbon coating enables the use of a variety of drugs and doses for preparing a drug-eluting stents. Translumina also makes the YUKON Choice drug-eluting stent using the PEARL surface, which enables the adsorption of different organic substances due to its mechanical modification. These non-polymer coatings are manufactured in a special designed cartridge in the Translumina Stent Coating Machine MAGIC BOX, which is especially designed for customized application of anti-proliferative, anti-inflammatory and/or anti-thrombotic drugs. See, for example, US Patent Application No. 2006/0124056. Another example of a non-polymeric stent coating is that described by GreatBatch (Clarence, N.Y.) whereby the vascular stent is composed of drug-eluting outer layer of a porous sputtered columnar metal having each column capped with a biocompatible carbon-containing material. See, for example, US Patent Application No. 2006/0200231. Another example of a non-polymeric stent coating is the bovine pericardium-covered stent made by Design and Performance Corp. (Richmond, BC, Canada). Chemical modification of the bovine pericardium can be performed to allow for its use in drug delivery to the vessel wall. See, for example, U.S. Pat. Nos. 7,108,717 and 6,929,658 and US Patent Application Nos. 2006/0206194; 2005/0278012; and 2005/0251244. Another example of a non-polymeric stent coating is the GENOUS Bio-engineered surface manufactured by Orbus Medical Technologies (Fort Lauderdale, Fla.). This coating has an antibody specific to the antigen cells that are in the blood thereby capturing the patient's circulating endothelial progenitor cells in order to accelerate the natural healing process. The GENOUS endothelial progenitor cell capture technology is designed to limit restenosis by quickly covering the stent with a layer of biocompatible endothelial cells. This coating is being used on Orbus Medical's R-STENT and may be optimized by incorporating a drug to the bio-engineered surface. See, for example, U.S. Pat. Nos. 7,108,714 and 7,037,332 and US Patent Application Nos. 2006/0135476; 2006/0121012 and 2005/0271701. Another example of a non-polymeric stent coating is CODRUG which is manufactured by Control Delivery Systems (Watertown, Mass.), which was recently acquired by pSivida Limited (Perth, Wash.). CODRUG is a non-linear drug delivery system that is a bioerodible polymer-free system that controls delivery over hours to weeks. This technology has been used on LEKTON MAGIC Absorbable Metal Stent made by Biotronik (Berlin, Germany). See, for example, US Patent Application Nos. 2005/0025834; 2005/0008695; 2004/0022853; 2003/0229390; 2003/0203030 and 2003/0158598. Another example of a non-polymeric stent coating is that being used for TAXCOR Drug-Eluting Stent made by EuroCOR GmbH (Bonn, Germany), which is a polymer-free system that uses attachment technology. In this technology, the compounds are loaded into microporous cavities (based on an open cellular fully carbonized stent surface). A protective layer of specific amino acid molecules avoids rapid drug elution and within 20 days provides for a moderate drug release to the vessel wall.

Stents may be combined with the compounds without a delivery system. For example, the ZILVER PTX self-expanding vascular stent manufactured by Cook Group Inc. (Bloomington, Ind.) utilizes the combination of the V-FLEX stainless steel coronary stent that is treated by a proprietary process with the drug itself such that the drug has direct contact with the vessel wall. This technology as well as other stent coating technologies from Cook are described in, for example, U.S. Pat. Nos. 6,918,927; 6,730,064; 6,530,951; 6,299,604 and 5,380,299.

Stents may be combined with a biomimetic system to help augment the stent's drug delivery capabilities. For example, Eucatech AG (Germany) makes a stent coating called the CAMOUFLAGE Coronary Stent System with athrombogenic properties based on the biomimicry of endothelial cell glycocalyx. CAMOUFLAGE has a carbohydrate backbone fragment that is covalently bound to the activated stent surface. Compounds may be incorporated into a biodegradable polymer matrix and then coated onto the CAMOUFLAGE ProActive Coating base layer, which is the basis for the EUCATAX Paclitaxel-Eluting Stent System that is being developed by Eucatech AG. Hemoteq (Germany) also makes a CAMOUFLAGE coating as well as polymeric drug delivery coatings, such as OUVERTURE, PROTEQTOR, REPULSION drug-eluting coatings. These coatings may be used in combination to provide a stent that with better drug delivery properties (e.g., the OUVERTURE coating is a combined coating of CAMOUFLAGE and REPULSION). See, for example, PCT Publication Nos. WO06116989; WO05039629 and WO03034944. Another example of a biomimetic stent coating is the polymer-free system that Biosensors International (Singapore) uses on its AXXION DES. The coating technology from Occam International (Netherlands) is based on its CALIX stent delivery system in which the drug is directly coated on the stent over a layer of glycocalix, a substrate designed to improve biocompatibility of the metal stent surface after the drug is released. This technology is also being used on the CUSTOM Nx Coronary Stent System manufactured by Xtent, Inc. (Menlo Park, Calif.). Another example of a biomimetic stent coating is the coating based on the bioactive peptide called P-15 which is a synthetic form of a natural molecule that is a major site of collagen activity. Cardiovasc (Menlo Park, Calif.) is developing a stent graft with a polymeric covering and P-15 which increases the coverage speed, adhesion and health of endothelial cells. See, for example, patent publication nos. WO0115764 and WO0182833.

Compounds may also be incorporated directly into the stent without a coating. For example, the IGAKI-TAMAI biodegradable drug-eluting stent is fabricated from polylactic acid to release a drug. This drug-eluting stent is made by Shiga Medical Center (Shiga, Japan) in collaboration with Igaki Medical Planning Company (Kyoto, Japan). See, for example, U.S. Pat. No. 5,733,327. Another example of a polymeric stent that delivers compounds directly is the coiled-shaped biodegradable temporary scaffold made of poly-L-lactic acid that serves to load compounds directly into the stent for gradual release to target tissues. This stent is described in, for example, U.S. Pat. No. 7,128,755. Another example of a polymeric stent is that described by Ethicon, which is composed of a biodegradable fiber having an inner core and an outer layer. The outer layer is a blend of two polymer components that have a degradation rate different from that of the inner layer. See, for example, the U.S. Pat. Nos. 6,537,312 and 6,423,091.

In addition to using the more traditional stents, stents that are specifically designed for drug delivery can be used. For example, Conor Medsystems (Menlo Park, Calif.) has created non-surface coated stents, whereby the stent incorporates hundreds of laser-drilled small holes, each acting as a reservoir into which drug-polymer compositions can be loaded. The reservoir design provides control drug release enabling a wider range of drug therapies. The drug reservoirs provide up to 16 times the drug volume of conventional surface-coated stents and permit a drug concentration gradient to be set up in each depot. The MEDSTENT is contoured and has ductile hinges allowing for the stent struts to be underformed during stent expansion and thus, holes created in these areas do not sacrificing strength, scaffolding or flexibility. Conor produces DEPOSTENT, MEDSTENT and COSTAR stents that may be used for drug delivery. Examples of these specialized drug delivery stents as well as traditional stents include those from Conor Medsystems, such as, for example, U.S. Pat. Nos. 6,527,799; 6,293,967; 6,290,673; 6,241,762; U.S. Patent Application Publication Nos. 2003/0199970 and 2003/0167085; and PCT Publication No. WO 03/015664. Another example of a specifically designed stent is the microporous covered stent that relies on nanotechnology and microfabrication processes developed by Advanced Bio Prosthetic Surfaces (San Antonio, Tex.). This is a molecular thin-film deposition system with struts and covers that are both hollow and microporous. The hollow struts act as reservoirs to contain compounds without the need for polymeric carriers. The system is designed for circumferential uniformity of elution directly into the vessel wall with flexibility in the type of compound used and the location of the reservoirs. The eNITINOL stent utilizes this type of technology. See, for example, U.S. Pat. Nos. 7,122,049 and 6,936,066; and US Patent Application No. 2005/0186241 and PCT Publication Nos. WO06015161 and WO02060506. Another example of a specifically designed stent is the CARBOSTENT made by Sorin Biomedica (Salugga, Italy) which has deep drug reservoirs covering the external stent surface and construction designed to optimize the mechanical response to stent expansion, flexture and torsion. After depositing a drug, the stent is covered with non-polymer CARBOFILM coating, which is designed to increase hemo- and biocompatibility. The JANUS CARBOSTENT and the TECNIC CARBOSTENT utilize this type of technology. See, for example, U.S. Pat. No. 6,699,281 and US Patent Application Nos. 2006/0030937; 2005/0209681 and 2004/0172124. Another example of a specifically designed stent is that described by Advanced Cardiovascular System whereby the stent has elements containing depots along the body structure that may contain therapeutic substances, polymeric substances, radioactive isotopes, radiopaque materials and/or any combination of thereof. See, for example, U.S. Pat. No. 7,060,093. Another example of a specifically designed stent is that described by Avantec Vascular Corp. (Sunnyvale, Calif.) which is an implantable scaffold having a substance reservoir present over at least a portion of the scaffold with a rate-controlling element formed over the substance-containing reservoir to provide for a number of different substance release characteristics. See, for example, U.S. Pat. No. 7,077,859.

The stent may be self-expanding or balloon expandable (e.g., STRECKER stent by Medi-Tech/Boston Scientific Corporation), or implanted by a change in temperature (e.g., nitinol stent). Self-expanding stents that can be used include the coronary WALLSTENT and the SCIMED RADIUS stent from Boston Scientific Corporation (Natick, Mass.) and the GIANTURCO stents from Cook Group, Inc. (Bloomington, Ind.). Examples of balloon expandable stents that can be used include the CROSSFLEX stent, BX-VELOCITY stent and the PALMAZ-SCHATZ crown and spiral stents from Cordis Corporation (Miami Lakes, Fla.), the V-FLEX PLUS stent by Cook Group, Inc., the NIR, EXPRESS and LIBERTE stents from Boston Scientific Corporation, the ACS MULTILINK, MULTILINK PENTA, SPIRIT, and CHAMPION stents from Guidant Corporation, and the Coronary Stent S670 and S7 by Medtronic, Inc. (Minneapolis, Minn.). Other examples of stents that can be combined with a combination of compounds in accordance with this disclosure include those from Boston Scientific Corporation, (e.g., the drug-eluting TAXUS EXPRESS² Paclitaxel-Eluting Coronary Stent System; over the wire stent stents such as the Express² Coronary Stent System and NIR Elite OTW Stent System; rapid exchange stents such as the EXPRESS² Coronary Stent System and the NIR ELITE MONORAIL Stent System; and self-expanding stents such as the MAGIC WALLSTENT Stent System and RADIUS Self Expanding Stent); Medtronic, Inc. (Minneapolis, Minn.) (e.g., DRIVER ABT578-eluting stent, DRIVER ZIPPER MX Multi-Exchange Coronary Stent System and the DRIVER Over-the-Wire Coronary Stent System; the S7 ZIPPER MX Multi-Exchange Coronary Stent System; S7, S670, S660, and BESTENT2 with Discrete Technology Over-the-Wire Coronary Stent System; ENDEAVOUR drug-eluting stent); Guidant Corporation (e.g., cobalt chromium stents such as the MULTI-LINK VISION Coronary Stent System; MULTI-LINK ZETA Coronary Stent System; MULTI-LINK PIXEL Coronary Stent System; MULTI-LINK ULTRA Coronary Stent System; and the MULTI-LINK FRONTIER); Johnson & Johnson/Cordis Corporation (e.g., CYPHER sirolimus-eluting Stent; PALMAZ-SCHATZ Balloon Expandable Stent; and S.M.A.R.T. Stents); Abbott Vascular (Redwood City, Calif.) (e.g., MATRIX LO Stent; ZOMAXX Drug-Eluting Stent; XIENCE V Everolimus Eluting Coronary Stent System; TRIMAXX Stent; and DEXAMET stent); AMG GmbH (Germany) (e.g., ARTHROS INERT carbonized stainless steel stent and ARTHROS PICO cobalt chromium stent); Biotronik (Switzerland) (e.g., MAGIC AMS stent); Clearstream Technologies (Ireland) (e.g., CLEARFLEX stent); Cook Inc. (Bloomington, Ind.) (e.g., V-FLEX PLUS stent, ZILVER PTX self-expanding vascular stent coating, LOGIX PTX stent (in development); Devax (Irvine, Calif.) (e.g., AXXESS Drug Eluting Stent); DISA Vascular (Pty) Ltd (South Africa) (e.g., CHROMOFLEX Stent, S-FLEX Stent, S-FLEX Micro Stent, and TAXOCHROME DES); Intek Technology (Baar, Switzerland) (e.g., APOLLO stent); Sorin Biomedica (Saluggia, Italy) (e.g., JANUS and CARBOSTENT); and stents from Bard/Angiomed GmbH Medizintechnik KG (Murray Hill, N.J.), and Blue Medical Supply & Equipment (Mariettta, Ga.), Millimed (Sweden) and Blue Medical (Netherlands) (e.g., XTRM-FIT Coronary Stent); Aachen Resonance GmbH (Germany) (e.g., FLEX FORCE Stent, VITASTENT); Eucatech AG (Germany) (EUCATAX Paclitaxel-Eluting stent system); EuroCOR GmbH (Bonn, Germany) (e.g., TAXCOR); Prot, Goodman, Terumo Corp. (Japan), (e.g., TSUNAMI Stent System); Translumina GmbH (Germany) (e.g., YUKON Choice drug-eluting stent); MIV Therapeutics (Canada), Occam International B.V. (Eindhoven, The Netherlands) (e.g., NEXUS stents); Sahajanand Medical Technologies PVT LTD. (India) (e.g., INFINNIUM Paclitaxel-Eluting Coronary Stent System, SUPRALIMUS Sirolimus Eluting Coronary Stent System, MILLENNIUM Matrix Coronary Stent System and CORONNIUM Cobalt Alloy Stent); AVI Biopharma/Medtronic/Interventional Technologies (Portland, Oreg.) (e.g., RESTEN NG-coated stent); Jomed (Sweden) (e.g., JOSTENT and FLEXMASTER Drug-Eluting Stent); MeoMedical GmbH (Germany)1 (e.g., MEO:FLEX and MEO:DRUGSTAR); Avantec Vascular (Sunnyvale, Calif.) (e.g., DURAFLEX Coronary Stent System); X-Cell Medical (Princeton, N.J.) (e.g., ETHOS Drug-Eluting Stent); and Atrium Medical (Hudson, N.H.) (e.g., FLYER Rx Coronary Stent).

Generally, stents are inserted in a similar fashion regardless of the site or the disease being treated. Briefly, a preinsertion examination, usually a diagnostic imaging procedure, endoscopy, or direct visualization at the time of surgery, is generally first performed in order to determine the appropriate positioning for stent insertion. A guidewire is then advanced through the lesion or proposed site of insertion, and over this is passed a delivery catheter which allows a stent in its collapsed form to be inserted. Intravascular stents may be inserted into an artery such as the femoral artery in the groin and advanced through the circulation under radiological guidance until they reach the anatomical location of the plaque in the coronary or peripheral circulation. Typically, stents are capable of being compressed, so that they can be inserted through tiny cavities via small catheters, and then expanded to a larger diameter once they are at the desired location. The delivery catheter then is removed, leaving the stent standing on its own as a scaffold. Once expanded, the stent physically forces the walls of the passageway apart and holds them open. A post insertion examination, usually an x-ray, is often utilized to confirm appropriate positioning.

Stents are typically maneuvered into place under, radiologic or direct visual control, taking particular care to place the stent precisely within the vessel being treated. In certain aspects, the stent can further include a radio-opaque, echogenic material, or MRI responsive material (e.g., MRI contrast agent) to aid in visualization of the device under ultrasound, fluoroscopy and/or magnetic resonance imaging. The radio-opaque or MRI visible material may be in the form of one or more markers (e.g., bands of material that are disposed on either end of the stent) that may be used to orient and guide the device during the implantation procedure.

### Intravascular Infusion Catheters and Drug-Delivery Catheters

In another aspect, the present disclosure provides for a combination of paclitaxel and dipyridamole (or analogues or derivatives thereof) or a composition comprising a combination of paclitaxel and dipyridamole (or analogues or derivatives thereof) and an intravascular catheter.

“Intravascular Catheter” refers to any a medical device having one or more lumens configured for the delivery of a formulation (e.g., aqueous, microparticulate, fluid, or gel formulations) into the bloodstream or into the vascular wall. These formulations may contain a combination of compounds described herein. Numerous intravascular catheters have been described for direct, site-specific drug delivery (e.g., microinjector catheters, catheters placed within or immediately adjacent to the target tissue), regional drug delivery (i.e., catheters placed in an artery that supplies the target organ or tissue), or systemic drug delivery (i.e., intra-arterial and intravenous catheters placed in the peripheral circulation). For example, catheters can deliver compounds from an end orifice, through one or more side ports, through a microporous outer structure, through one or multiple lumens, or through direct injection into the desired tissue or vascular location.

Catheters available for regional or localized intravascular drug-delivery include multilumen drug delivery catheters having a rigid collar with a plurality of apertures for implanting compounds into the lining of a vessel wall. See, for example, U.S. Pat. No. 5,180,366. Drug delivery catheters may have inner and outer shafts whereby the distal end has a plurality of grooved delivery area to expel drug to a vessel wall. See, for example, U.S. Pat. No. 5,904,670. The drug delivery catheter may have infusion arrays at the distal tip with many delivery conduits (LocalMed, Inc.) Drug is then introduced into the delivery passage and infused into the treatment site through the delivery orifices, as described in U.S. Pat. Nos. 5,941,868; 5,772,629 and 5,336,178. Other catheters have a support frame with a plurality of platforms that are deployed at the treatment site to expel drug from the platforms to the delivery interface for impregnation at the site as described in U.S. Pat. No. 5,279,565. Other catheters have fluid infusion tubes over a balloon surface to form isolated reservoir pockets for delivering drugs intraluminally. When the balloon is expanded, isolated reservoir pockets are formed between the tubes as described in U.S. Pat. No. 5,810,767.

The compounds described herein may be applied to the adventitial region using catheters, such as the MICROSYRINGE Infusion Catheter developed by Mercator Medsystems, Inc. (San Leandro, Calif.). This product is designed to deliver therapy directly to the adventitia of injured blood vessels where the inflammatory response occurs. The MICROSYRINGE catheter-guided, microfluid, infusion system is used as a site-specific delivery of compounds for applications to vascular disease. It acts to deliver drug directly into the vessel wall via endovascular catheter technology with a balloon-deployable microneedle. The microneedle slides through the vessel wall to deliver compounds when the balloon is deployed. Examples of catheters for delivery to an adventitial region are described in, for example, U.S. Pat. Nos. 7,127,284 and 7,070,606 and U.S. Published Patent Application Nos. 2006/0189941 and 2006/0111672.

In another aspect, a catheter designed for systemic intravascular drug delivery may be used to delivery the combination of compounds. For example, the catheter may have a multilumen for the delivery of fluids via a plurality of flow passageways and discharge openings in the wall of the outer tubular member. See, for example, U.S. Pat. No. 5,021,044. The Cragg-McNamara Valved Infusion Catheter available from Microtherapeutics, Inc. (San Clemente, Calif.) can be used to infuse biologically active agents without the use or requirement of a guidewire. The agents may be released through multi-side holes whose distribution of sizes or positions produces a variation in delivery rate and pressure of an agent over an infusion region.

In another aspect, drug delivery catheters may be used to locally deliver the described compounds liquid or non-liquid forms. For example, the compounds may be in the form of a pellet as described in U.S. Pat. No. 5,180,366. The compounds may be injected into the intramural site in the form of microparticles (with or without a polymeric carrier) as described in U.S. Pat. No. 5,171,217. The compounds may be in the form of a liquid which is held in a reservoir and expelled out the infusion port of a drug delivery catheter. See, for example, U.S. Pat. No. 6,200,257. The compounds may be in the form of a coating whereby the distal end of the catheter is coated with one or more layers of hydrogel copolymer wherein at least one layer of coating encapsulates medicaments. See, for example, U.S. Patent Application No. 2004/0220511.

Intravascular catheters can be used alone to deliver the combination of compounds or can be used together with balloons to provide a means to deliver the compounds into the walls of the vessel. These catheters have been enhanced and modified over the years to perform a variety of different applications. Types of catheters that may be used in drug delivery included, but are not limited to, passive-diffusion catheters, pressure-driven balloon catheters, mechanically-driven delivery catheters, and electrically enhanced delivery catheters.

The passive-diffusion catheter traps materials within an isolated segment or chamber whereby the compounds may be introduced through a separate port. The chamber is often created by the inflation of two balloons. The double-occlusion balloon is simple way to localize drug delivery to a site of interest without disrupting the vascular wall. An example of a double-occlusion balloon catheter is the DISPATCH balloon from Boston Scientific Corporation (Natick, Mass.). This device creates multiple chambers within a vessel segment through a nonporous membrane that spans the distance between the limbs of an inflatable coil. The drug may be infused for a long period of time in this type of delivery system since there is an inner polyurethane sheath that allows blood flow to continue unimpeded. This DISPATCH balloon catheter is a non-dilating local drug delivery system whereby drug is released through a series of drug spaces that are created by a spiral coil such that drug is isolated from blood flow and able to bathe the vessel wall. The delivery of drug in this system may be infused by a volume driven infusion pump or hand injection over a period of time (minutes to hours). See for example, Barsness et al. (2000), Amer. Heart Journal: 139(5): 824-9 and Glazier et al. (1997), Catheterization and Cardio. Diagnosis: 41(3): 261-7. Other double balloon drug delivery systems whereby medication may be released to the vessel wall are described, for example, in U.S. Pat. No. 5,049,132.

An example of another type of isolated segment passive diffusion catheter is the Stack Perfusion Coronary Dilatation catheters that are manufactured from Advanced Cardiovascular Systems, Inc. (Santa Clara, Calif.) as described in, for example, U.S. Pat. No. 5,195,971. These catheters have a primary perfusion port adjacent to the proximal end of the inflatable member and a transverse cross-sectional area to provide the bulk of the perfusion flow through the catheter.

The pressure-driven balloon catheters are based on a balloon on the distal end of the catheter that are inflated against the vessel wall that can either deliver drugs via perforations or via coating on the surface of the balloon. Examples of these types of catheters are the porous (WOLINSKY) balloons that are available from Advanced Polymers (Salem, N.H.), and are described in, e.g., U.S. Pat. No. 5,087,244. Another example is the CRESCENDO that is manufactured by Cordis Corporation (Miami Lakes, Fla.) is a modified perforated balloon that has an outer membrane with multiple pores to allow the drug to “weep” gently onto the endothelium of the target vessel as described in U.S. Pat. No. 5,318,531. These drug delivery balloons as well as other types are also described in more detail below.

Other pressure-driven balloon catheters include the infusion-sleeve catheter which consists of an outer sleeve with is loaded with drug and an inner balloon which is used to inflate the sleeve against the vessel wall. For example, Bavaria Medizin Technologie (Wessling, Germany) describes a sleeve catheter that supplies drug to the vessel wall through a number of outer lumina having radially discharge openings at the head portion of the catheter. This is slideable onto a balloon catheter so that it can be expanded to abut the inner wall of the vessel when dilated so that the medicament can be applied to a local area as described in U.S. Pat. No. 5,364,356. Other infusion sleeve catheters include the INFUSASLEEVE that is manufactured by LocalMed, Inc. (Sunnyvale, Calif.), which is a multilumen catheter consisting of a proximal infusion port, proximal hub, main catheter shaft, and distal infusion region with multiple side holes. The catheter has four separate outer lumens for drug delivery and side holes which are located within the infusion region near the distal tip of the infusion sleeve. The drug travels through the proximal infusion port and the outer infusion lumens and exits via side holes (nine 40-μm-diameter holes per drug-delivery lumen). The infusion sleeve is designed to track over standard dilatation balloon catheters and can be positioned relative to the balloon in one of three configurations. The infusion sleeve has been designed to provide independent control of the apposition of the drug-delivery elements against the arterial wall determined by the inflation pressure of the underlying PTCA balloon. Delivery of the compounds into the arterial wall is determined by the infusion pressure of the drug-delivery elements. This infusion sleeve is further described in U.S. Pat. Nos. 5,876,374; 5,840,008; and 5,634,901.

Catheters that mechanically enhance drug delivery use physical means to penetrate the endothelium to target the deeper layers of the internal vessel wall. For example, the INFILTRATOR catheter available from InterVentional Technologies, Inc. (San Diego, Calif.)) (see, e.g., U.S. Pat. No. 5,354,279) has needles or microport strips that run lengthwise on a dilation balloon. Since the catheter is pressure-driven, when the balloon is inflated it results in penetration of the needles into the target vessel wall. Because of the mechanical penetration of the needle, the delivery of the drug is high with very little washout. Catheters with needle-like probes at the distal end or through side openings whereby the probes penetrate the interior of the vessel wall for drug delivery are described, for example, in U.S. Pat. Nos. 6,302,870; 6,254,573; 6,197,013 and 6,183,444.

Catheters that electrically enhanced drug delivery are based on adapting a flowing electric current to the catheter to enhance the movement of drugs into the vessel wall. Electrophoretic and electro-osmotic enhancement may be utilized by coating the distal end of the catheter with a hydrogel composed of a drug and charged carriers to facilitate mobility of the drug to the vessel wall; as described, for example, in U.S. Patent Application No. 2004/0220511. There are also ultrasonically assisted (phonophoresis) and iontophoresis catheters, such as the GALILEO Centering Catheter from Guidant Corporation (Houston, Tex.), which is the first commercially available intravascular radiotherapy system. An iontophoresis system utilizing a double-walled, porous outer catheter for injecting drug into the vessel wall is described, for example, in U.S. Pat. No. 6,149,641. Other phonophoresis and iontophoresis catheters are described, for example, in Singh, J., et al. (1989) Drug Des. Deliv.: 4: 1-12 and U.S. Pat. Nos. 5,362,309; 5,318,014; 5,315,998; 5,304,120; 5,282,785; and 5,267,985.

Other catheter drug delivery systems are described, for example, by Riessen et al. (1994) JACC 23: 1234-1244, Kandarpa K. (2000) J. Vasc. Interv. Radio. 11 (suppl.): 419-423, and Yang, X. (2003) Imaging of Vascular Gene Therapy 228(1): 36-49.

Drug Delivery Balloons and Angioplasty Balloons

In one aspect, the present disclosure provides for a combination of paclitaxel and dipyridamole (or analogues or derivatives thereof) or a composition comprising a combination of paclitaxel and dipyridamole (or analogues or derivatives thereof) and a drug delivery balloon.

“Drug-Delivery Balloon” refers to a balloon device configured for insertion into an artery, such as a peripheral artery (typically the femoral artery). Drug delivery balloons may be based upon percutaneous angioplasty balloons which can be manipulated via a catheter to the treatment site (either in the coronary or peripheral circulation). Numerous drug delivery balloons have been developed for local delivery of compounds to the vascular (e.g., arterial) wall, including “sweaty balloons,” “channel balloons,” “microinjector balloons,” “double balloons,” “spiral balloons,” “balloon catheters” and other specialized drug-delivery balloons. Other examples of balloons include BHP balloons and Transurethral and Radiofrequency Needle Ablation (TUNA or RFNA)) balloons for prostate applications.

Intra-arterial balloons traditionally have been used to open up clogged blood vessels that are occluded with fatty plaque. In addition to the vascular system, intra-arterial balloons and catheters have been used to open constrictions and blockages due to scar tissue or neoplastic growth in other body cavities or tubes, such as, but not limited to the esophagus, biliary-duct, bronchi, urethra, ureter, fallopian-tubes, heart valves, tear-ducts and carpal tunnel dilatation.

In certain embodiments, the intra-arterial balloons are tightly wrapped around a catheter shaft to minimize its profile and are inserted into the vessel to the area of stenosis. Once in position, solution is forced through the catheter to inflate the balloon whereby the plaque is compressed against the wall of the vessel so that blood is allowed to flow normally. These intra-arterial balloons and associated catheters have been enhanced and modified over the years to perform a variety of different applications. For example, balloons have been shaped into specific shapes specific to their application and anatomical site. They can take on a series of different forms, such as, but not limited to, conical, spherical, elongated, dog-bone, offset, square, tapered, stepped, or any combination of these to form many other more complex shapes. The choice of the end form depends on the requirements of the end-use procedure. If required by the application, different ends can also be used on the same balloon.

Numerous drug delivery balloons have been developed for local delivery of compounds to the arterial wall or the wall of another body passageway. High-pressure balloons (i.e., catheters that apply force to expel medicaments) are one example of balloons that are used for drug delivery. Use of these types of balloons facilitates the localization of medicaments without unwanted systemic administration. Examples of high-pressure balloons include, but are not limited to “double balloons”, “sweaty balloons”, “channel balloons”, “microinjector balloons” and “spiral balloons”.

In one aspect of this disclosure, the compositions of this disclosures can be delivered into the treatment site and/or into the tissue surrounding the treatment site by using double balloons. Double balloons are high-pressure balloons using two discrete balloons mounted on a catheter shaft to seal off the afflicted area, while the medication is infused through a port in the catheter between the two balloons. Once the treatment is complete, the balloons are deflated and retracted. An example of a double-occlusion balloon catheter is the DISPATCH balloon from Boston Scientific Corporation (Natick, Mass.). The drug may be infused for a long period of time in this type of delivery system since there is an inner polyurethane sheath that allows blood flow to continue unimpeded. Other double balloon drug delivery systems whereby medication may be released to the vessel wall are described, for example, in U.S. Pat. Nos. 6,544,221 and 5,049,132.

In addition to the double-balloons, balloons may have a dog bone shape. Dogbone-shaped balloons can be used to deliver the described compounds by infusing the compounds through a series of holes in the narrower part of the balloon. The system can be guided into the desired location such that the inflatable bone-shaped balloon components are located on either side of the specific site that is to be treated.

The described compounds can be delivered into the treatment site and/or into the tissue surrounding the treatment site by using perforated or sweaty balloons. Sweaty balloons are perforated balloons that infuse compounds through microporous and/or macroporous holes or slits under high-pressure. When the balloon is inflated at the desired location, the desired compounds can be delivered through holes that are located in the balloon wall. The TRANSPORT catheter from Boston Scientific Corporation (Natick, Mass.), is an example of a perforated balloon that may be used to deliver drug to a target site. This catheter has a monorail design with a dual-layer balloon near the distal tip. There is a separate lumen that is used for inflation of the balloon, and a second lumen is used for drug infusion. This allowed uncoupling of the balloon support and drug delivery pressures. The outer balloon has microporous holes located circumferentially along the 10-mm-long mid-section of the balloon for controlled local drug delivery. Other representative examples of porous drug delivery balloons includes the WOLINSKY balloons, available from Advanced Polymers (Salem, N.H.), described in, e.g., U.S. Pat. No. 5,087,244. These balloons are ultra-thin-walled PET balloon which can be converted to a microporous membrane with hole sizes ranging from submicron to a few microns in diameter. A single balloon may contain hundreds of thousands or even millions of holes. By customizing the pore size, drug delivery can be controlled by enabling release of small amounts of a drug over a well-defined area. When the drug is released using this system, the balloon membrane “weeps” medication to form a thin film between the balloon membrane and the tissue forcing the medication into the vascular wall. Drug absorption and penetration into the vessel wall can be controlled by the rate of fluid flow across the membrane and the pressure at which the fluid is delivered. Other representative examples of these types of perforated balloons that may be used to deliver the compounds are described in U.S. Pat. Nos. 6,623,452; 5,397,307; 5,295,962; 5,286,254; 5,254,089; 5,087,244; 4,636,195 and 4,994,033 as well as PCT Publication No. WO 93/08866 and WO 92/11895 and in, e.g., Lambert, C. R. et al. (1992) Circ. Res. 71: 27-33.

In another aspect of this disclosure, the compositions of this disclosures can be delivered into the treatment site and/or into the tissue surrounding the treatment site by using channel balloons. Channel balloons are typically hollow, inflatable channel-like medication deliverable balloons at the distal end of a multi-lumen catheter. A plurality of conduits extend along the wall of the balloon for delivery of medicaments. Each conduit may include an array of closely spaced apertures for allowing medicaments in the conduit to transfer out of the conduits and into the surrounding vessel after the balloon is inflated. The REMEDY catheter from Boston Scientific Corporation is double-layer channeled perfusion balloon with intramural infusion channels that allow controlled, site-specific, targeted drug delivery independent of the inner dilation balloon pressure. This local delivery approach minimizes systemic toxicity while allowing high intramural drug concentration in the arterial wall at the site of balloon injury. In another example, the drug delivery balloon may be a single balloon infusion catheter that has an infusion chamber or pocket between the balloon and the vessel wall such that high concentrations of pharmaceutical formulations are delivered into the infusion chamber under low pressure for local infusion therapy during high pressure. See, for example, U.S. Pat. Nos. 5,833,658 and 5,558,642 and Buszman P et al. (2006) Kariol Pol.: 64(3): 268-274. Other representative examples of other channel balloons are described, for example, in U.S. Pat. Nos. 5,860,954; 5,843,033 and 5,254,089, and Hong, M. K., et al. (1992) Circulation: 86 Suppl. I: 1-380).

Compositions containing the paclitaxel and dipyridamole (or analogues or derivatives thereof) described herein can be delivered into the treatment site and/or into the tissue surrounding the treatment site by using catheter systems that have one or more injectors that can penetrate the surrounding tissue. These microinjector balloons typically contain a plurality of tubular fluid passageways that are longitudinally mounted on the balloon whereby a plurality of injectors are mounted on each tubular passageway and in fluid communication therewith. During use of the device, the balloon is first positioned in a vessel, and then inflated to embed the injectors into the vessel wall. The injector(s) are inserted into the desired location, for example by direct insertion into the tissue, by inflating the balloon or mechanical rotation of the injector, and the composition of this disclosure is injected into the desired location. Next, a fluid medicament is introduced through each of the fluid passageways for further infusion through the passageways and through the injectors into the vessel wall. For example, compounds may be delivered using a drug delivery balloon that has extensions that allow a rapid bolus infusion of a fluid to the deeper layers of the vessel wall. See, for example, U.S. Pat. No. 5,112,305. Representative examples of microinjector catheters that can be used for this application are described in and U.S. Patent Application Publication No. 2002/0082594 and U.S. Pat. Nos. 6,443,949; 6,488,659; 6,569,144; 5,746,716; 5,681,281; 5,609,151; 5,385,148; 5,551,427; 5,746,716; 5,681,281; and 5,713,863.

Compositions containing a combination of paclitaxel and dipyridamole (or analogues or derivatives thereof) can be delivered into the treatment site and/or into the tissue surrounding the treatment site by using spiral balloons. Typically, spiral and/or helical balloons are a series of flexible loops that inflate in a generally cooperative tubular shape. The loops may be supported by a coiled support member and may be configured to encourage tortuous compatibility between the catheter balloon arrangement and a body lumen. Helical patterned balloons having a plurality of elements around the support tube provides the ability to apply pressure via inflation while at the same time preserving blood flow in the blood vessel as well as side branches. For example, the drug delivery balloon may be an elongated tube with a lumen attached to an inflatable balloon with apertures that is helically wound through the elongated tube. As the balloon is inflated a sheath which is attached to the balloon forms containment pockets between the vessel wall and the balloon which allows perfusion of the drug solution. See, for example, U.S. Pat. No. 5,554,119. Other representative examples of spiral and helical balloons are described, for example, in U.S. Pat. Nos. 6,527,739; 6,605,056; 6,190,356; 5,279,546; 5,236,424, 5,226,888; 5,181,911; 4,824,436; and 4,636,195.

The compositions of this disclosure can be delivered using a catheter that has the ability to enhance uptake or efficacy of the compositions of this disclosure. The stimulus for enhanced uptake can include the use of heat, the use of cooling, the use of electrical fields or the use of radiation (e.g., ultraviolet light, visible light, infrared, microwaves, ultrasound or X-rays). Further representative examples of catheter systems that can be used are described in U.S. Pat. Nos. 5,362,309 and 6,623,444; U.S. Patent Application Publication Nos. 2002/0138036 and 2002/0068869; and PCT Publication Nos. WO 01/15771; WO 94/05361; WO 96/04955 and WO 96/22111.

A catheter may be adapted to deliver a thermoreversible polymer composition. For the site-specific delivery of these materials, a catheter delivery system has the ability to either heat the composition to above body temperature or to cool the composition to below body temperature such that the composition remains in a fluent state within the catheter delivery system. The catheter delivery system can be guided to the desired location and the composition of this disclosure can be delivered to the surface of the surrounding tissue or can be injected directly into the surrounding tissue. A representative example of a catheter delivery system for direct injection of a thermoreversible material is described in U.S. Pat. No. 6,488,659. Representative examples of catheter delivery systems that can deliver the thermoreversible compositions to the surface of the vessel are described in U.S. Pat. Nos. 6,443,941; 6,290,729; 5,947,977; 5,800,538; and 5,749,922.

The compositions of this disclosure may be delivered into the treatment site and/or into the tissue surrounding the treatment site by using a coating method. Once a compound is coated onto the catheter balloon, it can be released using pressure, heat, or laser light. For example, laser and thermal energy have been used experimentally to enhance binding of heparin to an injured arterial wall. In the experiment, lesions were treated successfully after angioplasty with a laser balloon that had been coated with heparin. Alternatively, pressure release of drugs from a coated balloon is also effective which is the method used for the ULTRATHIN GLIDES from Boston Scientific Corporation (see, e.g., Fram, D. B. et al. (1992) Circulation: 86 Suppl. I: 1-380). In another example, drug delivery balloons may be coated with a hydrogel carrying drum which is squeezed by the balloon against the vessel wall upon inflation. The hydrogel coating is a tenaciously adhered swellable hydrogel polymer containing a preselected drug which is released during compression against the vessel wall thereby coating the wall of the body lumen. See, for example, U.S. Pat. No. 5,304,121.

In another aspect, paclitaxel and dipyridamole (or analogues or derivatives thereof) may be directly coated onto the surface of the balloon without a polymer. For example, Bavarian Medical Therapies (Germany) is conducting early stage clinical studies using PACCOCATH, a drug-coated balloon coated with paclitaxel. This paclitaxel-coated balloon technology allows for drug delivery to the total injured vessel area, with or without stent implantation, and therefore, may be used in the treatment of in-stent restenosis as an alternative to brachytherapy or stent-in-stent applications. The drug may be coated onto a conventional angioplasty balloon by spraying paclitaxel onto its surface using acetone as the solvent as well as a hydrophilic x-ray contrast-medium substance. When the balloon is inflated, the drug is transferred from the balloon to the vessel wall. These types of drug delivery balloons are described in U.S. Patent Application No. 2006/0020243. Other representative examples of drug delivery balloons that use the coating technology are described, for example, in PCT Publication No, WO 92/11890.

Anastomotic Connector Devices

In another aspect, the present disclosure provides for a combination of paclitaxel and dipyridamole (or analogues or derivatives thereof) or a composition comprising a combination of paclitaxel and dipyridamole (or analogues or derivatives thereof) and an anastomotic connector device.

“Anasomotic connector device” refers to any vascular device that mechanizes the creation of a vascular anastomosis (i.e., artery-to-artery, vein-to-artery, artery-to-vein, artery-to-synthetic graft, synthetic graft-to-artery, vein-to-synthetic graft or synthetic graft-to-vein anastomosis) without the manual suturing that is typically done in the creation of an anastomosis. The term also refers to anastomotic connector devices (described below), designed to produce a facilitated semiautomatic vascular anastomosis without the use of suture and reduce connection time substantially (often to several seconds), where there are numerous types and designs of such devices. The term also refers to devices which facilitate attachment of a vascular graft to an aperture or orifice (e.g., in the side or at the end of a vessel) in a target vessel. Anastomotic connector devices may be anchored to the outside of a blood vessel, and/or into the wall of a blood vessel (e.g., into the adventitial, intramural, or intimal layer of the tissue), and/or a portion of the device may reside within the lumen of the vessel.

Anastomotic connector devices may be used to create new flow from one structure to another through a channel or diversionary shunt. Accordingly, such devices (also referred to herein as “bypass devices”) typically include at least one tubular structure, wherein a tubular structure defines a lumen. Anastomotic connector devices may include one tubular structure or a plurality of tubular structures through which blood can flow. At least a portion of the tubular structure resides external to a blood vessel (i.e., extravascular) to provide a diversionary passageway. A portion of the device also may reside within the lumen and/or within the tissue of the blood vessel.

Examples of anastomotic connector devices are described in co-pending application entitled, “Anastomotic Connector Devices”, filed May 23, 2003 (U.S. Ser. No. 60/473,185). Broadly, anastomotic connector devices may be classified into three categories: (1) automated and modified suturing methods and devices, (2) micromechanical devices, and (3) anastomotic coupling devices. Representative examples of anastomotic connector devices include, without limitation, vascular clips, vascular sutures, vascular staples, vascular clamps, suturing devices, anastomotic coupling devices (i.e., anastomotic couplers), including couplers that include tubular segments for carrying blood, anastomotic rings, percutaneous in situ coronary artery bypass (PISCAB and PICVA) devices.

Automated sutures and modified suturing methods generally facilitate the rapid deployment of multiple sutures or a suture clip, usually in a single step, and eliminate the need for knot tying or the use of aortic side-biting clamps. Automated and modified suturing methods and devices also have been developed to deliver a vascular graft to complete an anastomosis.

Suturing devices include those devices that are adapted to be minimally invasive such that anastomoses are formed between vascular conduits and hollow organ structures by applying sutures or other surgical fasteners through device ports or other small openings. With these devices, sutures and other fasteners are applied in a relatively quick and automated manner within bodily areas that have limited access. By using minimally invasive means for establishing anastomoses, there is less blood loss and there is no need to temporarily stop the flow of blood distal to the operating site. For example, the suturing device may be composed of a shaft-supported vascular conduit that is adapted for anastomosis and a collar that is slideable on the shaft configured to hold a plurality of needles and sutures that passes through the vascular conduit. See, e.g., U.S. Pat. No. 6,709,441. The suturing device may be composed of a carrier portion for inserting graft, arm portions that extend to support the graft into position, and a needle assembly adapted to retain and advance coil fasteners into engagement with the vessel wall and the graft flange to complete the anastomosis. See, e.g., U.S. Pat. No. 6,709,442. The suturing device may include two oblong interlinked members that include a split bush adapted for suturing (e.g., U.S. Pat. No. 4,350,160).

Micromechanical devices are used to create an anastomosis and/or secure a graft vessel to the site of an anastomosis. Representative examples of micromechanical devices include staples (either penetrating or non-penetrating) and clips.

Anastomotic coupling devices may be used to connect a first blood vessel to a second vessel, either with or without a graft vessel, for completion of an anastomosis. In one aspect, anastomotic coupling devices facilitate automated attachment of a graft or vessel to an aperture or orifice (e.g., in the side or at the end of a vessel) in a target vessel without the use of sutures or staples.

Anastomotic coupling devices may comprise a tubular structure defining a lumen through which blood may flow (described below). These types of devices (also referred to herein as “bypass devices”) can function as an artificial passageway or conduit for fluid communication between blood vessels and can be used to divert (i.e., shunt) blood from one part of a blood vessel (e.g., an artery) to another part of the same vessel, or to a second vessel (e.g., an artery or a vein) or to multiple vessels (e.g., a vein and an artery).

Bypass devices may be used in a variety of end-to-end and end-to-side anastomotic procedures. The bypass device may be placed into a patient where it is desired to create a pathway between two or more vascular structures, or between two different parts of the same vascular structure. For example, bypass devices may be used to create a passageway which allows blood to flow around a blood vessel, such as an artery (e.g., coronary artery, carotid artery, or artery supplying the lower limb), which has become damaged or completely or partially obstructed. Bypass devices may be used in coronary artery bypass surgery to shunt blood from an artery, such as the aorta, to a portion of a coronary artery downstream from an occlusion in the artery.

Certain types of anastomotic coupling devices are configured to join two abutting vessels. The device can further include a tubular segment to shunt blood to another vessel. These types of connectors are often used for end-to-end anastomosis if a vessel is severed or injured.

Introduction of an anastomotic connector into or onto an intramural, luminal, or adventitial portion of a blood vessel may irritate or damage the endothelial tissue of the blood vessel and/or may alter the natural hemodynamic flow through the vessel. This irritation or damage may stimulate a cascade of biological events resulting in a fibrotic response which can lead to the formation of scar tissue in the vessel. Incorporation of a combination of compounds in accordance with this disclosure into or onto a portion of the device that is in direct contact with the blood vessel (e.g., a terminal portion or edge of the device) may inhibit scarring, making the vessel less prone to the formation of intimal hyperplasia and stenosis.

Thus, in one aspect, the compounds may be associated only with the portion of the device that is in contact with the blood or endothelial tissue. For example, the compounds may be incorporated into only an intravascular portion (i.e., that portion that resides within the lumen of the vessel or in the vessel tissue) of the device. The compounds may be incorporated onto all or a portion of the intravascular portion of the device. In other embodiments, the coating may reside on all or a portion of an extravascular portion of the device.

As intravascular devices are made in a variety of configurations and sizes, the exact dose of the administered compounds will vary with device size, surface area and design. Regardless of the method of application of the compounds to the intravascular device, the total amount (dose) of each compound in or on the device may be in the range of about 0.01 μg-10 μg, or 10 μg-10 mg, or 10 mg-250 mg, or 250 mg-1000 mg, or 1000 mg-2500 mg. The dose (amount) of each compound per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

In certain aspects, intravascular devices (e.g., intravascular stents) are provided that are associated with a combination of paclitaxel and dipyridamole, where the total amount of each compound on, in or near the device may be in an amount ranging from less than 0.01 μg to about 2500 μg per mm² of device surface area. Generally, the compound may be in an amount ranging from less than 0.01 μg; or from 0.01 μg to about 1.0 μg; or from 0.01 μg to about 10 μg; or from about 0.5 μg to about 5 μg; or from about 0.05 μg to 50 μg; or from 10 μg to about 250 μg; or from 250 μg to about 2500 μg (per mm² of device surface area).

In certain aspects, intravascular devices (e.g., vascular stents) are provided in which paclitaxel may be present in an amount ranging from about 0.01 to about 1.0 μg/mm² or from about 0.1 to about 0.6 μg/mm² and dipyridamole is present in an amount ranging from about 0.05 to about 50 μg/mm² or from about 0.5 to about 5 μg/mm².

The total amount of each compound made available on, in or near the device may be in an amount ranging from about 0.01 μg (micrograms) to about 2500 mg (milligrams). Generally, the compounds may be in the amount ranging from 0.01 μg to about 10 μg; or from 10 μg to about 1 mg; or from 1 mg to about 10 mg; or from 10 mg to about 100 mg; or from 100 mg to about 500 mg; or from 500 mg to about 2500 mg.

In certain embodiments, intravascular devices (e.g., vascular stents) are provided that are combined with paclitaxel in an amount ranging from about 10 to about 60 μg and dipyridamole in an amount ranging from about 120 to about 170 μg.

In certain embodiments, intravascular devices (e.g., vascular stents) are provided that are combined with paclitaxel in an amount ranging from about 30 to about 50 μg and dipyridamole in an amount ranging from about 140 to about 160 μg.

In certain aspects, the weight ratio of dipyridamole to paclitaxel may be adjusted to provide a superior biological effect (e.g., to minimize formation of neointimal hyperplasia). In one embodiment, the weight ratio of dipyridamole to paclitaxel may exceed about 0.06 to about 1.0 to provide a superior biological effect. In other embodiments, the weight ratio of dipyridamole to paclitaxel may be adjusted to exceed about 0.06; or about 0.08; or about 0.10; or about 0.20; or about 0.30 or about 0.40; or about 0.50; or about 0.60; or about 0.70; or about 0.80; or about 0.90; or about 1.0; or about 1.1; or about 1.2; or about 1.3; or about 1.4; or about 1.5; or about 1.6.

Vena Cava Filters

In one aspect, the present disclosure provides for a combination of compounds as described herein and an inferior vena cava filter device. Inferior vena cava filters are devices intended to capture emboli and prevent them from migrating through the blood stream. Examples of vena cava filters include, without limitation, vascular filters, blood filters, implantable blood filters, caval filters, inferior vena cava filters, vena cava filtering devices, thrombosis filters, thrombus filters, antimigration filters, filtering devices, percutaneous filter systems, intravascular traps, intravascular filters, clot filters, vein filters and body vessel filters.

Inferior vena cava filters catch blood clots to prevent them from traveling to other parts of the body to form an embolus. It may be life threatening if plaques or blood clots migrate through the blood stream and travel to the lungs and cause a pulmonary embolism. To prevent such an occurrence, inferior vena cava filters are placed in the large veins of the body to prevent pulmonary emboli in patients with (or at risk of developing) deep vein thrombosis. Most often these filters are composed of synthetic polymers or metals. These filters may be a variety of configurations, including but not limited to, baskets, cones, umbrellas or loops. The shape of the filter must provide adequate trapping ability while allowing sufficient blood flow. Along with the functional shape, filters may also have other design features including peripheral loops for alignment or anchoring features to prevent migration (e.g., ridges, struts or sharp points). Where the filter comes into contact with the vessel wall for anchoring, a fibrotic response may occur. This fibrotic response can result in difficulties in removal of the filter. This is a particular problem for filters that are to be kept in place for a relatively short period of time. Incorporation of a combination of compounds as described herein into or onto the filter may reduce or prevent stenosis or obstruction of the device via a fibroproliferative response.

In one aspect, inferior vena cava filters may be designed in a variety of configurations. For example, the inferior vena cava filter may be composed of a plurality of intraluminal filter elements held by a retainer in a filter configuration that may be released to an open, stent-like configuration. See, e.g., U.S. Pat. No. 6,267,776. The inferior vena cava filter may be composed of an embolus capturing portion having a plurality of elongated filter wires diverging in a helical arrangement to form a conical surface and an anchoring portion that has a plurality of struts. See, e.g., U.S. Pat. No. 6,391,045. The inferior vena cava filter may be composed of a textured echogenic feature so the filter position may be determined by sonographic visualization. See, e.g., U.S. Pat. No. 6,436,120. The inferior vena cava filter may be composed of a plurality of core wire struts that are anchored to radiate outwardly which are interconnected by compression material to form a filter basket. See, e.g., U.S. Pat. No. 5,370,657. The inferior vena cava filter may be composed of an apical head with a plurality of divergent legs in a conical shaped geometry which have a hook and pad for securing to the vessel. See, e.g., U.S. Pat. No. 5,059,205. The inferior vena cava filter may be composed of a filtering device made of shape memory/superelastic material formed at the distal end of a deployment/retrieval wire section for minimally invasive positioning. See, e.g., U.S. Pat. No. 5,893,869. The inferior vena cava filter may be composed of a plurality of intraluminal elements joined by a retainer, whereby upon release of the retainer, the intraluminal filter elements convert to an open configuration in the blood vessel. See, e.g., U.S. Pat. Nos. 6,517,559 and 6,267,776. The inferior vena cava filter may be composed of an outer catheter and an inner catheter having a collapsible mesh-like filter basket at the distal end made of spring wires or plastic monofilaments. See, e.g., U.S. Pat. No. 5,549,626. The inferior vena cava filter may be composed of a plurality of radiating struts that attach at a body element and has a two layer surface treatment to provide endothelial cell growth and anti-proliferative properties. See, e.g., U.S. Pat. No. 6,273,901. The inferior vena cava filter may be composed of a metal fabric that is configured as a particle-trapping screen that may be slideable along a guidewire. See, e.g., U.S. Pat. No. 6,605,102. The inferior vena cava filter may be non-permanent with a single high memory coiled wire having a cylindrical and a conical segment. See, e.g., U.S. Pat. No. 6,059,825. Other inferior vena cava filters are described in, e.g., U.S. Pat. Nos. 6,623,506; 6,391,044; 6,231,589; 5,984,947; 5,695,518 and 4,817,600.

Vena cava filters, which may be combined with one or more a combination of compounds according to the present disclosure, include commercially available products. Examples of vena cava filters include, without limitation, the GÜNTHER TULIP Vena Cava FILTER and the GIANTURCO-ROEHM BIRD'S NEST Filter which are sold by Cook, Inc. (Bloomington, Ind.). C.R. Bard (Murray Hill, N.J.) sells the SIMON-NITINOL FILTER and RECOVERY Filter. Cordis Endovascular which is a subsidiary of Cordis Corporation (Miami Lakes, Fla.) sells the TRAPEASE Permanent Vena Cava Filter. B. Braun Medical Inc. (Bethlehem, Pa.) sells the VENA TECH LP Vena Cava Filter and VENA TECH-LGM Vena Cava Filter. Boston Scientific Corporation (Natick, Mass.) sells the Over-the-Wire GREENFIELD Vena Cava Filter.

As vena cava filters are made in a variety of configurations sizes and include a variety of different materials, the exact dose of the administered compounds will vary with device size, composition, surface area and design. Regardless of the method of application of the compounds to the device, the total amount (dose) of each compound in or on the device may be in the range of about 0.01 μg-10 μg, or 10 μg-10 mg, or 10 mg-250 mg, or 250 mg-1000 mg, or 1000 mg-2500 mg. The dose (amount) of each compound per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

In certain aspects, vena cava filter devices are provided that are associated with a combination of paclitaxel and dipyridamole, where the total amount of each compound on, in or near the device may be in an amount ranging from less than 0.01 μg to about 2500 μg per mm² of device surface area. Generally, the compound may be present in an amount ranging from less than 0.01 μg; or from 0.01 μg to about 1.0 μg; or from 0.01 μg to about 10 μg; or from about 0.5 μg to about 5 μg; or from about 0.05 μg to 50 μg; or from 10 μg to about 250 μg; or from 250 μg to about 2500 μg (per mm² of device surface area).

In certain embodiments, paclitaxel is present in an amount ranging from about 0.01 to about 1.0 μg/mm² and dipyridamole is present in an amount ranging from about 0.05 to about 50 μg/mm².

In other embodiments, paclitaxel is present in an amount ranging from about 0.1 to about 0.6 μg/mm² and dipyridamole is present in an amount ranging from about 0.5 to about 5 μg/mm².

The total amount of each compound made available on, in or near the device may be in an amount ranging from about 0.01 μg (micrograms) to about 2500 mg (milligrams). Generally, the compounds may be in the amount ranging from 0.01 μg to about 10 μg; or from 10 μg to about 1 mg; or from 1 mg to about 10 mg; or from 10 mg to about 100 mg; or from 100 mg to about 500 mg; or from 500 mg to about 2500 mg.

In one embodiment, the weight ratio of dipyridamole to paclitaxel may exceed about 0.06 to about 1.0 to provide a superior biological effect. The weight ratio of dipyridamole to paclitaxel may be adjusted to exceed about 0.06; or about 0.08; or about 0.10; or about 0.20; or about 0.30 or about 0.40; or about 0.50; or about 0.60; or about 0.70; or about 0.80; or about 0.90; or about 1.0; or about 1.1; or about 1.2; or about 1.3; or about 1.4; or about 1.5; or about 1.6.

Gastrointestinal Stents

The present disclosure provides for the combination of paclitaxel and dipyridamole (or analogues or derivatives thereof) and a gastrointestinal (GI) stent.

The term “GI stent” refers to devices that are located in the gastrointestinal tract including the biliary duct, pancreatic duct, colon, and the esophagus. GI stents are or comprise scaffoldings that are used to treat endoluminal body passageways that have become blocked due to disease or damage, including malignancy or benign disease.

In one aspect, the GI stent may be an esophageal stent used to keep the esophagus open whereby food is able to travel from the mouth to the stomach. For example, the esophageal stent may be composed of a cylindrical supporting mesh inner layer, retaining mesh outer layer and a semi-permeable membrane sandwiched between. See, e.g., U.S. Pat. No. 6,146,416. The esophageal stent may be a radially, self-expanding stent of open weave construction with an elastomeric film formed along the stent to prevent tissue ingrowth and distal cuffs that resist stent migration. See, e.g., U.S. Pat. No. 5,876,448. The esophageal stent may be composed of a flexible wire configuration to form a cylindrical tube with a deformed end portion increased to a larger diameter for anchoring pressure. See, e.g., U.S. Pat. No. 5,876,445. The esophageal stent may be a flexible, self-expandable tubular wall incorporating at least one truncated conical segment along the longitudinal axis. See, e.g., U.S. Pat. No. 6,533,810.

In another aspect, the GI stent may be a biliary stent used to keep the biliary duct open whereby bile is able to drain into the small intestines. For example, the biliary stent may be composed of shape memory alloy. See, e.g., U.S. Pat. No. 5,466,242. The biliary stent may be a plurality of radially extending wings with grooves which project from a helical core. See, e.g., U.S. Pat. Nos. 5,776,160 and 5,486,191.

In another aspect, the GI stent may be a colonic stent. For example, the colonic stent may be a hollow tubular body that may expand radially and be secured to the inner wall of the organ in a release fitting. See, e.g., European Patent Application No. EP1092400A2.

In another aspect, the GI stent may be a pancreatic stent used to keep the pancreatic duct open to facilitate secretion into the small intestines. For example, the pancreatic stent may be composed of a soft biocompatible material which is resiliently compliant which conforms to the duct's curvature and contains perforations that facilitates drainage. See, e.g., U.S. Pat. No. 6,132,471.

GI stents, which may be combined with one or more compounds according to the present disclosure, include commercially available products, such as the NIR Biliary Stent System and the WALLSTENT Endoprostheses from Boston Scientific Corporation (Natick, Mass.). Other commercially available products include the PALMAZ-SCHATZ Transhepatic Biliary Stent (Cordis (Miami, Fla.), the Biliary Endoprostheses from Edwards Lifesciences (Irvine, Calif.), DYNALINK (Guidant, St. Paul, Minn.); COOK-Z Stent and the ZA-STENT Endoscopic Biliary Stent System (Wilson-Cook Medical, Winston-Salem, N.C.).

As GI stents are made in a variety of configurations and sizes, the exact dose of the administered compounds will vary with device size, surface area and design. Regardless of the method of application of the compounds to the device, the total amount (dose) of each compound in or on the device may be in the range of about 0.01 μg-10 μg, or 10 μg-10 mg, or mg-250 mg, or 250 mg-1000 mg, or 1000 mg-2500 mg. The dose (amount) of each compound per unit area of device surface to which the agent is applied may be in the range of about 0.01 μg/mm²-1 μg/mm², or 1 μg/n=2-10 μg/mm², or 10 μg/mm²-250 μg/mm², 250 μg/mm²-1000 μg/mm², or 1000 μg/mm²-2500 μg/mm².

In certain aspects, GI stent devices are provided that are associated with a combination of paclitaxel and dipyridamole, where the total amount of each compound on, in or near the device may be in an amount ranging from less than 0.01 μg to about 2500 μg per mm² of device surface area. Generally, the compound may be present in an amount ranging from less than 0.01 μg; or from 0.01 μg to about 1.0 μg; or from 0.01 μg to about 10 μg; or from about 0.5 μg to about 5 μg; or from about 0.05 μg to 50 μg; or from 10 μg to about 250 μg; or from 250 μg to about 2500 μg (per mm² of device surface area).

In certain embodiments, paclitaxel is present in an amount ranging from about 0.01 to about 1.0 μg/mm² and dipyridamole is present in an amount ranging from about 0.05 to about 50 μg/mm².

In other embodiments, paclitaxel is present in an amount ranging from about 0.1 to about 0.6 μg/mm² and dipyridamole is present in an amount ranging from about 0.5 to about 5 μg/mm².

The total amount of each compound made available on, in or near the device may be in an amount ranging from about 0.01 μg (micrograms) to about 2500 mg (milligrams). Generally, the compounds may be in the amount ranging from 0.01 μg to about 10 μg; or from 10 μg to about 1 mg; or from 1 mg to about 10 mg; or from 10 mg to about 100 mg; or from 100 mg to about 500 mg; or from 500 mg to about 2500 mg.

In one embodiment, the weight ratio of dipyridamole to paclitaxel may exceed about 0.06 to about 1.0 to provide a superior biological effect. The weight ratio of dipyridamole to paclitaxel may be adjusted to exceed about 0.06; or about 0.08; or about 0.10; or about 0.20; or about 0.30 or about 0.40; or about 0.50; or about 0.60; or about 0.70; or about 0.80; or about 0.90; or about 1.0; or about 1.1; or about 1.2; or about 1.3; or about 1.4; or about 1.5; or about 1.6.

Tracheal and Bronchial Stents

The present disclosure provides for a combination of paclitaxel and dipyridamole (or analogues or derivatives thereof) and a tracheal or bronchial stent device.

Representative examples of tracheal or bronchial stents that can benefit from being coated with or having incorporated therein, a combination of the described compounds include tracheal stents or bronchial stents, including metallic and polymeric tracheal or bronchial stents and tracheal or bronchial stents that have an external covering (e.g., polyurethane, poly(ethylene terephthalate), PTFE, or silicone rubber).

Tracheal and bronchial stents may be, for example, composed of an elastic plastic shaft with metal clasps that expands to form a lumen along the axis for opening the diseased portion of the trachea and having three sections to emulate the natural shape of the trachea. See, e.g., U.S. Pat. No. 5,480,431. The tracheal/bronchial stent may be a T-shaped tube having a tracheotomy tubular portion that projects outwardly through a tracheotomy orifice which is configured to close and form a fluid seal. See, e.g., U.S. Pat. Nos. 5,184,610 and 3,721,233. The tracheal/bronchial stent may be composed of a flexible, synthetic polymeric resin with a tracheotomy tube mounted on the wall with a bifurcated bronchial end that is configured in a T-Y shape with specific curves at the intersections to minimize tissue damage. See, e.g., U.S. Pat. No. 4,795,465. The tracheal/bronchial stent may be a scaffolding configured to be substantially cylindrical with a shape-memory frame having geometrical patterns and having a coating of sufficient thickness to prevent epithelialization. See, e.g., U.S. Patent Application Publication No. 2003/0024534A1.

Tracheal/bronchial stents, which may be combined with one or more compounds according to the present disclosure, include commercially available products, such as the WALLSTENT Tracheobronchial Endoprostheses and ULTRAFLEX Tracheobronchial Stent Systems from Boston Scientific Corporation, the DUMON Tracheobronchial Silicone Stents from Bryan Corporation (Woburn, Mass.) and the DYNAMIC Tracheal Stent from Rusch (Germany).

Another type of device for use in the lung is a tubular conduit that includes a grommet portion, such as are described in, for example, U.S. Pat. No. 6,629,951 (to Broncus Technologies, Inc.). These devices maintain collateral openings or channels through the airway wall so that expired air is able to pass directly out of the lung tissue and may be used in the treatment of COPD and emphysema.

As tracheal/bronchial are made in a variety of configurations sizes and include a variety of different materials, the exact dose of the administered compounds will vary with device size, composition, surface area and design. Regardless of the method of application of the compounds to the device, the total amount (dose) of each compound in or on the device may be in the range of about 0.01 μg-10 μg, or 10 μg-10 mg, or 10 mg-250 mg, or 250 mg-1000 mg, or 1000 mg-2500 mg. The dose (amount) of each compound per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

In certain aspects, tracheal and bronchial stent devices are provided that are associated with a combination of paclitaxel and dipyridamole, where the total amount of each compound on, in or near the device may be in an amount ranging from less than 0.01 μg to about 2500 μg per mm² of device surface area. Generally, the compound may be present in an amount ranging from less than 0.01 μg; or from 0.01 μg to about 1.0 μg; or from 0.01 μg to about 10 μg; or from about 0.5 μg to about 5 μg; or from about 0.05 μg to 50 μg; or from 10 μg to about 250 μg; or from 250 μg to about 2500 μg (per mm² of device surface area).

In certain embodiments, paclitaxel is present in an amount ranging from about 0.01 to about 1.0 μg/mm² and dipyridamole is present in an amount ranging from about 0.05 to about 50 μg/mm².

In other embodiments, paclitaxel is present in an amount ranging from about 0.1 to about 0.6 μg/mm² and dipyridamole is present in an amount ranging from about 0.5 to about 5 μg/mm².

The total amount of each compound made available on, in or near the device may be in an amount ranging from about 0.01 μg (micrograms) to about 2500 mg (milligrams). Generally, the compounds may be in the amount ranging from 0.01 μg to about 10 μg; or from 10 μg to about 1 mg; or from 1 mg to about 10 mg; or from 10 mg to about 100 mg; or from 100 mg to about 500 mg; or from 500 mg to about 2500 mg.

In one embodiment, the weight ratio of dipyridamole to paclitaxel may exceed about 0.06 to about 1.0 to provide a superior biological effect. The weight ratio of dipyridamole to paclitaxel may be adjusted to exceed about 0.06; or about 0.08; or about 0.10; or about 0.20; or about 0.30 or about 0.40; or about 0.50; or about 0.60; or about 0.70; or about 0.80; or about 0.90; or about 1.0; or about 1.1; or about 1.2; or about 1.3; or about 1.4; or about 1.5; or about 1.6.

Genital-Urinary Stents

The present disclosure provides for a combination of paclitaxel and dipyridamole (or analogues or derivatives thereof) and genital-urinary (GU) stent device.

Representative examples genital-urinary (GU) stents that can benefit from being coated with or having incorporated therein, a combination of the described compounds include ureteric and urethral stents, fallopian tube stents, prostate stents, including metallic and polymeric GU stents and GU stents that have an external covering (e.g., polyurethane, poly(ethylene terephthalate), PTFE or silicone rubber).

In one aspect, genital-urinary stents include ureteric and urethral stents. Ureteral stents are hollow tubes with holes along the sides and coils at either end to prevent migration. Ureteral stents are used to relieve obstructions (caused by stones or malignancy), to facilitate the passage of stones, or to allow healing of ureteral anastomoses or leaks following surgery or trauma. They are placed endoscopically via the bladder or percutaneously via the kidney.

Urethral stents are used for the treatment of recurrent urethral strictures, detruso-external sphincter dyssynergia and bladder outlet obstruction due to benign prostatic hypertrophy. In addition, procedures that are conducted for the prostate, such as external radiation or brachytherapy, may lead to fibrosis due to tissue insult resulting from these procedures. The incidence of urethral stricture in prostate cancer patients treated with external beam radiation is about 2%. Development of urethral stricture may also occur in other conditions such as following urinary catheterization or surgery, which results in damage to the epithelium of the urethra. The clinical manifestation of urinary tract obstruction includes decreased force and caliber of the urinary stream, intermittency, postvoid dribbling, hesitance and nocturia. Complete closure of the urethra can result in numerous problems including eventual kidney failure. To maintain patency in the urethra, urethral stents may be used. The stents are typically self-expanding and composed of metal superalloy, titanium, stainless steel or polyurethane.

For example, the ureteric/urethral stent may be composed of a main catheter body of flexible polymeric material having an enlarged entry end with a hydrophilic tip that dissolves when contacted with body fluids. See, e.g., U.S. Pat. No. 5,401,257. The ureteric/urethral stent may be composed of a multi-sections including a closed section at that the bladder end which does not contain any fluid passageways such that it acts as an anti-reflux device to prevent reflux of urine back into the kidney. See, e.g., U.S. Pat. No. 5,647,843. The ureteric/urethral stent may be composed of a central catheter tube made of shape memory material that forms a stent with a retention coil for anchoring to the ureter. See, e.g., U.S. Pat. No. 5,681,274. The ureteric/urethral stent may be a composed of an elongated flexible tubular stent with preformed set curls at both ends and an elongated tubular rigid extension attached to the distal end which allows the combination function as an externalized ureteral catheter. See, e.g., U.S. Pat. Nos. 5,221,253 and 5,116,309. The ureteric/urethral stent may be composed of an elongated member, a proximal retention structure, and a resilient portion connecting them together, whereby they are all in fluid communication with each other with a slideable portion providing a retracted and expanded position. See, e.g., U.S. Pat. No. 6,685,744. The ureteric/urethral stent may be a hollow cylindrical tube that has a flexible connecting means and locating means that expands and selectively contracts. See, e.g., U.S. Pat. No. 5,322,501. The ureteric/urethral stent may be composed of a stiff polymeric body that affords superior columnar and axial strength for advancement into the ureter, and a softer bladder coil portion for reducing the risk of irritation. See, e.g., U.S. Pat. No. 5,141,502. The ureteric/urethral stent may be composed of an elongated tubular segment that has a pliable wall at the proximal region and a plurality of members that prevent blockage of fluid drainage upon compression. See, e.g., U.S. Pat. No. 6,676,623. The ureteric/urethral stent may be a catheter composed of a conduit which is part of an assembly that allows for non-contaminated insertion into a urinary canal by providing a sealing member that surrounds the catheter during dismantling. See, e.g., U.S. Patent Application Publication No. 2003/0060807A1.

In another aspect, genital-urinary stents include prostatic stents. For example, the prostatic stent may be composed of two polymeric rings constructed of tubing with a plurality of connecting arm members connecting the rings in a parallel manner. See, e.g., U.S. Pat. No. 5,269,802. The prostatic stent may be composed of thermoplastic material and a circumferential reinforcing helical spring, which provides rigid mechanical support while being flexible to accommodate the natural anatomical bend of the prostatic urethra. See, e.g., U.S. Pat. No. 5,069,169.

In another aspect, genital-urinary stents include fallopian stents and other female genital-urinary devices. For example, the genital-urinary device may be a female urinary incontinence device composed of a vaginal-insertable supporting portion that is resilient and flexible, which is capable of self-support by expansion against the vaginal wall and extending about the urethral orifice. See, e.g., U.S. Pat. No. 3,661,155. The genital-urinary device may be a urinary evacuation device composed of a ovular bulbous concave wall having an opening to a body engaging perimetal edge integral with the wall and an attached tubular member with a pleated body. See, e.g., U.S. Pat. No. 6,041,448.

Genital-urinary stents, which may be combined with paclitaxel and dipyridamole (or analogues or derivatives thereof) according to the present disclosure, include commercially available products, such as the UROLUME Endoprosthesis Stents from American Medical Systems, Inc. (Minnetonka, Minn.), the RELIEVE Prostatic/Urethral Endoscopic Device from InjecTx, Inc. (San Jose, Calif.), the PERCUFLEX Ureteral Stents from Boston Scientific Corporation, and the TARKINGTON Urethral Stents, FIRLIT-KLUGE Urethral Stents from Cook Group Inc (Bloomington, Ind.), and the SPANNER Prostatic Stent from AbbeyMoor Medical (Miltona, Minn.).

As GU stents are made in a variety of configurations and sizes, the exact dose of the administered compounds will vary with device size, surface area and design. Regardless of the method of application of the compounds to the device, the total amount (dose) of each compound in or on the device may be in the range of about 0.01 μg-10 μg, or 10 μg-10 mg, or 10 mg-250 mg, or 250 mg-1000 mg, or 1000 mg-2500 mg. The dose (amount) of each compound per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

In certain aspects, GU stent devices are provided that are associated with a combination of paclitaxel and dipyridamole, where the total amount of each compound on, in or near the device may be in an amount ranging from less than 0.01 μg to about 2500 μg per mm² of device surface area. Generally, the compound may be present in an amount ranging from less than 0.01 μg; or from 0.01 μg to about 1.0 μg; or from 0.01 μg to about 10 μg; or from about 0.5 μg to about 5 μg; or from about 0.05 μg to 50 μg; or from 10 μg to about 250 μg; or from 250 μg to about 2500 μg (per mm² of device surface area).

In certain embodiments, paclitaxel is present in an amount ranging from about 0.01 to about 1.0 μg/mm² and dipyridamole is present in an amount ranging from about 0.05 to about 50 μg/mm².

In other embodiments, paclitaxel is present in an amount ranging from about 0.1 to about 0.6 μg/mm² and dipyridamole is present in an amount ranging from about 0.5 to about 5 μg/mm².

The total amount of each compound made available on, in or near the device may be in an amount ranging from about 0.01 μg (micrograms) to about 2500 mg (milligrams). Generally, the compounds may be in the amount ranging from 0.01 μg to about 10 μg; or from 10 μg to about 1 mg; or from 1 mg to about 10 mg; or from 10 mg to about 100 mg; or from 100 mg to about 500 mg; or from 500 mg to about 2500 mg.

In one embodiment, the weight ratio of dipyridamole to paclitaxel may exceed about 0.06 to about 1.0 to provide a superior biological effect. The weight ratio of dipyridamole to paclitaxel may be adjusted to exceed about 0.06; or about 0.08; or about 0.10; or about 0.20; or about 0.30 or about 0.40; or about 0.50; or about 0.60; or about 0.70; or about 0.80; or about 0.90; or about 1.0; or about 1.1; or about 1.2; or about 1.3; or about 1.4; or about 1.5; or about 1.6.

Ear and Nose Stents

The present disclosure provides for a combination of paclitaxel and dipyridamole (or analogues or derivatives thereof) and an ear-nose-throat (ENT) stent device (e.g., a lacrimal duct stent, Eustachian tube stent, nasal stent, or sinus stent).

The sinuses are four pairs of hollow regions contained in the bones of the skull named after the bones in which they are located (ethmoid, maxillary, frontal and sphenoid). All are lined by respiratory mucosa which is directly attached to the bone. Following an inflammatory insult such as an upper respiratory tract infection or allergic rhinitis, a purulent form of sinusitis can develop. Occasionally secretions can be retained in the sinus due to altered ciliary function or obstruction of the opening (ostea) that drains the sinus. Incomplete drainage makes the sinus prone to infection typically with Haemophilus influenza, Streptococcus pneumoniae, Moraxella catarrhalis, Veillonella, Peptococcus, Corynebacterium acnes and certain species of fungi.

When initial treatment such as antibiotics, intranasal steroid sprays and decongestants are ineffective, it may become necessary to perform surgical drainage of the infected sinus. Surgical therapy often involves debridement of the ostea to remove anatomic obstructions and removal of parts of the mucosa. Occasionally a stent (a cylindrical tube which physically holds the lumen of the ostea open) is left in the osta to ensure drainage is maintained even in the presence of postoperative swelling. ENT stents, typically made of stainless steel or plastic, remain in place for several days or several weeks before being removed. It should be noted that similar effects can be achieved via infusion of paclitaxel and dipyridamole (or analogues or derivatives thereof) via a catheter or administration via a balloon inserted to open the sinus.

Representative examples of ENT stents that can benefit from being coated with or having incorporated therein the compounds described herein include lacrimal duct stents, Eustachian tube stents, nasal stents, and sinus stents.

The ENT stent may be a choanal atresia stent composed of two long hollow tubes that are bridged by a flexible transverse tube. See, e.g., U.S. Pat. No. 6,606,995. The ENT stent may be an expandable nasal stent for postoperative nasal packing composed of a highly porous, pliable and absorbent foam material capable of expanding outwardly, which has a nonadherent surface. See, e.g., U.S. Pat. No. 5,336,163. The ENT stent may be a nasal stent composed of a deformable cylinder with a breathing passageway that has a smooth outer non-absorbent surface used for packing the nasal cavity following surgery. See, e.g., U.S. Pat. No. 5,601,594. The ENT stent may be a ventilation tube composed of a flexible, plastic, tubular vent with a rectangular flexible flange which is used for the nasal sinuses following endoscopic antrostomy. See, e.g., U.S. Pat. No. 5,246,455. The ENT stent may be a ventilating ear tube composed of a shaft and an extended tab which is used for equalizing the pressure between the middle ear and outer ear. See, e.g., U.S. Pat. No. 6,042,574. The ENT stent may be a middle ear vent tube composed of a non-compressible, tubular base and an eccentric flange. See, e.g., U.S. Pat. No. 5,047,053. ENT stents, which may be combined with the compounds according to the present disclosure, include commercially available products such as Genzyme Corporation (Ridgefield, N.J.) SEPRAGEL Sinus Stents, the MEROGEL Nasal Dressing and Sinus Stents from Medtronic Xomed Surgical Products, Inc. (Jacksonville, Fla.), the POLYFLEX Stent from Rusch (Germany), and the FREEMAN Frontal Sinus Stent from InHealth Technologies (Carpinteria, Calif.). Other exemplary products which may be combined with the compounds described include the RELIEVA Balloon Sinuplasty (Acclarent Inc., Menlo Park, Calif.) catheter-based devices made of flexible tubes with a balloon on the distal end. These devices are configured to track over the sinus guidewire to the blocked ostium, which is then gradually inflated to gently restructure the ostium and are intended for clearing blocked sinuses, restoring normal sinus drainage and function, and preserving normal anatomy and mucosal tissue. See, for example, US Patent Applications 2006/0210605; 2006/0063973; and 2006/0095066.

As ENT stents are made in a variety of configurations and sizes, the exact dose of the administered compounds will vary with device size, surface area and design. Regardless of the method of application of the compounds to the device, the total amount (dose) of each compound in or on the device may be in the range of about 0.01 μg-10 μg, or 10 μg-10 mg, or mg-250 mg, or 250 mg-1000 mg, or 1000 mg-2500 mg. The dose (amount) of each compound per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

In certain aspects, ENT stent devices are provided that are associated with a combination of paclitaxel and dipyridamole, where the total amount of each compound on, in or near the device may be in an amount ranging from less than 0.01 μg to about 2500 μg per mm² of device surface area. Generally, the compound may be present in an amount ranging from less than 0.01 μg; or from 0.01 μg to about 1.0 μg; or from 0.01 μg to about 10 μg; or from about 0.5 μg to about 5 μg; or from about 0.05 μg to 50 μg; or from 10 μg to about 250 μg; or from 250 μg to about 2500 μg (per mm² of device surface area).

In certain embodiments, paclitaxel is present in an amount ranging from about 0.01 to about 1.0 μg/mm² and dipyridamole is present in an amount ranging from about 0.05 to about 50 μg/mm².

In other embodiments, paclitaxel is present in an amount ranging from about 0.1 to about 0.6 μg/mm² and dipyridamole is present in an amount ranging from about 0.5 to about 5 μg/mm².

The total amount of each compound made available on, in or near the device may be in an amount ranging from about 0.01 μg (micrograms) to about 2500 mg (milligrams). Generally, the compounds may be in the amount ranging from 0.01 μg to about 10 μg; or from 10 μg to about 1 mg; or from 1 mg to about 10 mg; or from 10 mg to about 100 mg; or from 100 mg to about 500 mg; or from 500 mg to about 2500 mg.

In one embodiment, the weight ratio of dipyridamole to paclitaxel may exceed about 0.06 to about 1.0 to provide a superior biological effect. The weight ratio of dipyridamole to paclitaxel may be adjusted to exceed about 0.06; or about 0.08; or about 0.10; or about 0.20; or about 0.30 or about 0.40; or about 0.50; or about 0.60; or about 0.70; or about 0.80; or about 0.90; or about 1.0; or about 1.1; or about 1.2; or about 1.3; or about 1.4; or about 1.5; or about 1.6.

Vascular Grafts

In one aspect, the present disclosure provides for a combination of paclitaxel and dipyridamole (or analogues or derivatives thereof) and a vascular graft.

The vascular graft may be an extravascular graft or an intravascular (i.e., endoluminal) graft. The vascular graft may be, without limitation, in the form of a peripheral bypass application or a coronary bypass application. Vascular grafts may be used to replace or substitute damaged or diseased veins and arteries, including, without limitation, blood vessels damaged by aneurysms, intimal hyperplasia and thrombosis. Vascular grafts may also be used to provide access to blood vessels, for example, for hemodialysis access. Vascular grafts are implanted, for example, to provide an alternative conduit for blood flow through damaged or diseased areas in veins and arteries, including, without limitation, blood vessels damaged by aneurysms, intimal hyperplasia and thrombosis, however, the graft may lead to further complications, including, without limitation, infections, inflammation, thrombosis and intimal hyperplasia. The lack of long-term patency with vascular grafts may be due, for example, to surgical injury and abnormal hemodynamics and material mismatch at the suture line. Typically, further disease (e.g., restenosis) of the vessel occurs along the bed of the artery.

Representative examples of vascular grafts include, without limitation, synthetic bypass grafts (e.g., femoral-popliteal, femoral-femoral, axillary-femoral, and the like), vein grafts (e.g., peripheral and coronary), and internal mammary (e.g., coronary) grafts, bifurcated vascular grafts, intraluminal grafts, endovascular grafts and prosthetic grafts. Synthetic grafts can be made from a variety of polymeric materials, such as, for example, polytetrafluoroethylene (e.g., ePTFE), polyesters such as DACRON, polyurethanes, and combinations of polymeric materials. In one embodiment, the synthetic vascular graft is formed of a porous synthetic material such as expanded PTFE (ePTFE).

Other forms of vascular grafts which may be used include those that (a) use a Miller cuff, which is a small piece of natural vein to make a short cuff that is joined by stitching it to the artery opening and the prosthetic graft; (b) use a flanged graft whereby the graft has a terminal skirt or cuff that facilitates an end-to-side anastomosis; (c) use a graft with an enlarged chamber having a large diameter for suture at the anastomosis site; and (d) use a graft that dispensing an agent that prevents thrombosis and/or intimal hyperplasia.

Vascular grafts, which may be combined with one or more agents according to the present disclosure, include commercially available products such as the LIFESPAN line of ePTFE vascular grafts from Edwards Lifesciences (Irvine, Calif.). Other examples of commercially available materials include GORE-TEX Vascular Grafts and GORE-TEX INTERING Vascular Grafts are sold by Gore Medical Division (W. L. Gore & Associates, Inc. Newark, Del.). C.R. Bard, Inc. (Murray Hill, N.J.) sells the DISTAFLO Bypass Grafts and IMPRA CARBOFLO Vascular Grafts. Atrium Medical (Hudson, N.H.) makes the ADVANTA family of PTFE vascular grafts. Atrium also makes other non-PTFE grafts, such as FLIXENE (Atrium Medical), which is a composite graft construction designed to minimize “weeping” often seen with traditional vascular bypass grafts following implantation, and the ULTRAMAX gel impregnated vascular grafts (also made by Atrium Medical).

As vascular grafts are made in a variety of configurations and sizes, the exact dose of the administered compounds will vary with device size, surface area and design. Regardless of the method of application of the compounds to the device, the total amount (dose) of each compound in or on the device may be in the range of about 0.01 μg-10 μg, or 10 μg-10 mg, or mg-250 mg, or 250 mg-1000 mg, or 1000 mg-2500 mg. The dose (amount) of each compound per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

In certain aspects, vascular grafts are provided that are associated with a combination of paclitaxel and dipyridamole, where the total amount of each compound on, in or near the device may be in an amount ranging from less than 0.01 μg to about 2500 μg per mm² of device surface area. Generally, the compound may be present in an amount ranging from less than 0.01 μg; or from 0.01 μg to about 1.0 μg; or from 0.01 μg to about 10 μg; or from about 0.5 μg to about 5 μg; or from about 0.05 μg to 50 μg; or from 10 μg to about 250 μg; or from 250 μg to about 2500 μg (per mm² of device surface area).

In certain embodiments, paclitaxel is present in an amount ranging from about 0.01 to about 1.0 μg/mm² and dipyridamole is present in an amount ranging from about 0.05 to about 50 μg/mm².

In other embodiments, paclitaxel is present in an amount ranging from about 0.1 to about 0.6 μg/mm² and dipyridamole is present in an amount ranging from about 0.5 to about 5 μg/mm².

The total amount of each compound made available on, in or near the device may be in an amount ranging from about 0.01 μg (micrograms) to about 2500 mg (milligrams). Generally, the compounds may be in the amount ranging from 0.01 μg to about 10 μg; or from 10 μg to about 1 mg; or from 1 mg to about 10 mg; or from 10 mg to about 100 mg; or from 100 mg to about 500 mg; or from 500 mg to about 2500 mg.

In one embodiment, the weight ratio of dipyridamole to paclitaxel may exceed about 0.06 to about 1.0 to provide a superior biological effect. The weight ratio of dipyridamole to paclitaxel may be adjusted to exceed about 0.06; or about 0.08; or about 0.10; or about 0.20; or about 0.30 or about 0.40; or about 0.50; or about 0.60; or about 0.70; or about 0.80; or about 0.90; or about 1.0; or about 1.1; or about 1.2; or about 1.3; or about 1.4; or about 1.5; or about 1.6.

Hemodialysis Access Devices

In one aspect, the present disclosure provides for the combination of paclitaxel and dipyridamole (or analogues or derivatives thereof) and a hemodialysis access device. Hemodialysis dialysis access devices that include a combination of compounds as described herein may be capable of inhibiting or reducing the overgrowth of granulation tissue, which can improve the clinical efficacy of these devices.

Hemodialysis access devices may be used when blood needs to be removed, cleansed and then returned to the body. Hemodialysis regulates the body's fluid and chemical balances as well as removes waste from the blood stream that cannot be cleansed by a normally functioning kidney due to disease or injury. For hemodialysis to occur, the blood may be obtained through a hemodialysis access or vascular access, in which minor surgery is performed to provide access through an AV fistula or AV access graft. These hemodialysis access devices may develop complications, including infections, inflammation, thrombosis and intimal hyperplasia of the associated blood vessels. The lack of long-term patency with hemodialysis access may be due to surgical injury, abnormal hemodynamics and material mismatch at the suture line. Typically, further disease (e.g., restenosis) of the vessel occurs along the bed of the artery and/or at the site of anastomosis.

In addition to the AV fistulas and AV access grafts described above, implantable subcutaneous hemodialysis access systems such as the commercially available catheters, ports, and shunts, may also be used for hemodialysis patients. These access systems may consist of a small metallic or polymeric device or devices implanted underneath the skin. These devices may be connected to flexible tubes, which are inserted into a vessel to allow for blood access.

Representative examples of hemodialysis access devices include, without limitation, AV access grafts, venous catheters, vascular grafts, a catheter system or a device used for an AV fistula, an implantable access port, a shunt (e.g., AV shunt), or a valve.

Synthetic hemodialysis access devices can be made from metals or polymers, such as polytetrafluoroethylene (e.g., ePTFE), polyesters such as DACRON, polyurethanes, or combinations of these materials.

Hemodialysis access devices, which may be combined with one or more agents according to the present disclosure, include commercially available products. For example, hemodialysis access devices include products, such as the LIFESITE (Vasca Inc., Tewksbury, Mass.) and the DIALOCK catheters from Biolink Corp. (Middleboro, Mass.), VECTRA Vascular Access Grafts and VENAFLO Vascular Grafts from C.R. Bard, Inc. (Murray Hill, N.J.), and GORE-TEX Vascular Grafts; Stretch Vascular Grafts from Gore Medical Division (W. L. Gore & Associates, Inc. Newark, Del.); and the LIFESPAN line of ePTFE vascular grafts from Edwards Lifesciences (Irvine, Calif.).

As hemodialysis access devices are made in a variety of configurations and sizes, the exact dose of the administered compounds will vary with device size, surface area and design. Regardless of the method of application of the compounds to the device, the total amount (dose) of each compound in or on the device may be in the range of about 0.01 μg-10 μg, or 10 μg-10 mg, or 10 mg-250 mg, or 250 mg-1000 mg, or 1000 mg-2500 mg. The dose (amount) of each compound per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

In certain aspects, hemodialysis access devices are provided that are associated with a combination of paclitaxel and dipyridamole, where the total amount of each compound on, in or near the device may be in an amount ranging from less than 0.01 μg to about 2500 μg per mm² of device surface area. Generally, the compound may be present in an amount ranging from less than 0.01 μg; or from 0.01 μg to about 1.0 μg; or from 0.01 μg to about 10 μg; or from about 0.5 μg to about 5 μg; or from about 0.05 μg to 50 μg; or from 10 μg to about 250 μg; or from 250 μg to about 2500 μg (per mm² of device surface area).

In certain embodiments, paclitaxel is present in an amount ranging from about 0.01 to about 1.0 μg/mm² and dipyridamole is present in an amount ranging from about 0.05 to about 50 μg/mm².

In other embodiments, paclitaxel is present in an amount ranging from about 0.1 to about 0.6 μg/mm² and dipyridamole is present in an amount ranging from about 0.5 to about 5 μg/mm².

The total amount of each compound made available on, in or near the device may be in an amount ranging from about 0.01 μg (micrograms) to about 2500 mg (milligrams). Generally, the compounds may be in the amount ranging from 0.01 μg to about 10 μg; or from 10 μg to about 1 mg; or from 1 mg to about 10 mg; or from 10 mg to about 100 mg; or from 100 mg to about 500 mg; or from 500 mg to about 2500 mg.

In one embodiment, the weight ratio of dipyridamole to paclitaxel may exceed about 0.06 to about 1.0 to provide a superior biological effect. The weight ratio of dipyridamole to paclitaxel may be adjusted to exceed about 0.06; or about 0.08; or about 0.10; or about 0.20; or about 0.30 or about 0.40; or about 0.50; or about 0.60; or about 0.70; or about 0.80; or about 0.90; or about 1.0; or about 1.1; or about 1.2; or about 1.3; or about 1.4; or about 1.5; or about 1.6.

Perivascular Devices

In one aspect, the present disclosure provides for a combination of paclitaxel and dipyridamole (or analogues or derivatives thereof) and a perivascular device. Incorporation of a combination of compounds into or onto a perivascular device may minimize fibrosis (or scarring) in the vicinity of the implant and have other related advantages. In certain aspects, the device may be used to deliver one or more of the compounds to the adjacent tissue (e.g., as a perivascular delivery device for the prevention of neointimal hyperplasia at an anastomotic site).

The device may take a variety of forms. In one aspect, be in the form of a surgical sheet which is in the form of a film or a fabric (e.g., textiles and meshes). Other forms for the materials include, for example, membranes (e.g., barrier membranes), surgical patches, surgical wraps (e.g., vascular, perivascular, adventitial, periadventitital wraps, peritubular, and adventitial sheets), bandages, surgical dressings, gauze, tapes, polymer shells, torroidal devices, annular devices, tissue coverings, and other types of surgical matrices, scaffolds, sheets, rings, collars, slabs, cuffs, membrane and sheaths.

In one aspect, the device comprises or may be in the form of a film. The film may be formed into one of many geometric shapes. Depending on the application, the film may be formed into the shape of a tube or may be a thin, elastic sheet of polymer. Generally, films are less than 5, 4, 3, 2, or 1 mm thick, more preferably less than 0.75 mm, 0.5 mm, 0.25 mm, or, 0.10 mm thick. Films can also be generated of thicknesses less than 50 μM, 25 μm or 10 μm. Films generally are flexible with a good tensile strength (e.g., greater than 50, preferably greater than 100, and more preferably greater than 150 or 200 N/cm²), good adhesive properties (i.e., adheres to moist or wet surfaces), and have controlled permeability. Films may be non-porous or porous (e.g., perforated) and may be configured for application to the surface of a tissue, cavity or an organ or may be applied to of a device or implant as well as to the surface.

Films may be made by various processes, including for example, by casting, and by spraying, or may be formed at the treatment site in situ. For example, a sprayable formulation may be applied onto the treatment site which then forms into a solid film. Additional materials, such as fibers or particles, may be incorporated into the film during its manufacture to alter the physical or chemical characteristics of the film (e.g., to enhance the strength of the material) or to modulate release of the described compounds from the film (e.g., a film may be loaded with particles containing a combination of compounds).

In one aspect, devices for perivascular applications may be constructed of a plurality of fibers (i.e., a fibrous construct or material), where the fibers are arranged in such a manner (e.g., interwoven, knotted, braided, overlapping, looped, knitted, interlaced, intertwined, webbed, felted, and the like) so as to form a porous structure. A fibrous construct may include fibers or filaments that are randomly oriented relative to each other or that are arranged in an ordered array or pattern. Preferably, a fibrous construct has intertwined threads that form a porous structure. Examples of fibrous materials include textiles, knitted, braided, crocheted, woven, non-woven (e.g., a melt-blown or wet-laid) or webbed fabrics, meshes, sheets, or gauzes. The fabric may be made from a natural or synthetic polymer which has been formed into a mesh material, such as a knit mesh, a weave mesh, a sprayed mesh, a web mesh, a braided mesh, a looped mesh, and the like. In certain embodiments of this disclosure, the described compounds are provided in systems which include knitted fabrics (e.g., meshes).

In certain embodiments, the devices are made from a pliable material having sufficient flexibility to conform to the particular anatomical structure at the implant site and typically possess physical characteristics, which make them useful as peritubular or perivascular drug delivery platforms. For example, the device may be a relatively flat material (e.g., a sheet), which may remain substantially flat after implantation, or it may be re-configured to conform to the geometry of the tissue at the site of implantation. The flat material may take a variety of forms. For example, the flat material may be configured as a single layer of material having perpendicular edges (e.g., a rectangle or square); may be circular or triangular in shape. Alternatively, the flat material may be in the form of a tube (e.g., a knitted tube) or other shape, which has been pressed flat.

As noted above, devices are provided that may include a fibrous material which is formed of or comprises fibers (also referred to herein as “yarn”). Each fiber may be constructed from one filament or a plurality of filaments (also referred to herein as “strands”). The number and type of filaments can be tailored impart the yarn with a range of different physical properties, depending on the specific application. The diameter and length of the fibers or filaments may range in size depending on the form of the material (e.g., knit, woven, or non-woven), and the desired elasticity, porosity, surface area, flexibility, and tensile strength. The fibers may be of any length, ranging from short filaments to long threads (i.e., several microns to hundreds of meters in length).

Fibers having dimensions appropriate for preparing fibrous constructs (e.g., knit fabrics) may be made using standard melt-processing techniques, such as injection molding, compression molding, extrusion, electrospinning, melt spinning, solution spinning and gel state spinning.

The fibrous construct generally possesses sufficient porosity to permit the flow of fluids through the pores of the fiber network and to facilitate tissue ingrowth and/or fluid flow. Generally, the interstices of the fibrous material should be sufficiently wide apart to allow light visible by eye, or fluids, to pass through the pores. However, materials having a more compact structure also may be used.

Perivascular materials may be used in a variety of surgical procedures (described in more detail below), e.g., bypass graft procedures, that result in the flow of blood from a high flow vessel (e.g., an artery) into a low flow vessel (e.g., a vein), oftentimes through a bypass graft. Due to significant discrepancy between blood flow rate and pressure in these two vessel types, the increased blood flow through the vein may cause the vein to expand in size to accommodate the increased blood volume. Perivascular devices may benefit having a degree of elasticity are capable of expanding in the days or weeks following implantation to accommodate the increase in vein size without constricting the vein.

Perivascular materials are typically flexible materials that are capable of being wrapped around all or a portion of the external surface of a body passageway or cavity. For example, materials may be used as a perivascular wrap, which can be wrapped, either fully or partially, about a blood vessel. As such, the materials are typically in the form of woven or knitted sheets having a thickness ranging from about 25 microns to about 3000 microns; preferably from about 50 to about 1000 microns. Materials suitable for wrapping around arteries and veins typically have thicknesses which range from about 100 to 600 microns. In certain embodiments, the material has a thickness of less than 500 microns; or less than 400 microns; or less than 300 microns; or less than 200 microns.

The device may be formed from a polymer, which may be biodegradable or non-biodegradable. In some aspects, the polymer may be a bioresorbable, biodegradable polymer (e.g., a naturally derived and synthetic biodegradable polymer).

Representative examples of naturally derived polymers include albumin, collagen, hyaluronic acid and derivatives, sodium alginate and derivatives, chitosan and derivatives gelatin, starch, cellulose polymers (e.g., methylcellulose, hydroxypropylcellulose, hydroxypropylmethylcellulose, carboxymethylcellulose, cellulose acetate phthalate, cellulose acetate succinate, hydroxypropylmethylcellulose phthalate), casein, dextran and derivatives, polysaccharides, and fibrinogen.

Synthetic biodegradable polymers and copolymers may be formed from one or more cyclic monomers (e.g., D-lactide, L-lactide, D,L-lactide, meso-lactide, glycolide, ε-caprolactone, trimethylene carbonate (TMC), p-dioxanone (e.g., 1,4-dioxane-2-one or 1,5-dioxepan-2-one), or a morpholinedione).

In certain embodiments, the device include polymer fibers that comprise a plurality of glycolide and lactide (e.g., L-lactide, D-lactide, or mixtures thereof, also referred to as D,L-lactide) residues or meso-lactide). The ratio of glycolide to lactide residues in the copolymer may be varied depending on the desired properties of the fiber. For example, the polymer may have a molar ratio of glycolide residues that is greater than about 80; or greater than about 85; or greater than about 90; or greater than about 95. The fiber may be formed from a polymer having a 3:97 molar ratio of lactide (e.g., D,L-lactide) to glycolide, or a 5:95 molar ratio of lactide to glycolide, or a 10:90 molar ratio of lactide to glycolide.

Additional examples of polymeric materials include poly(D,L-lactic acid), poly(L-lactic acid) oligomers and polymers, poly(D-lactic acid) oligomers and polymers, poly(glycolic acid)), and copolymers of lactic acid and glycolic acid), poly(hydroxyvaleric acid), poly(malic acid), and poly(tartronic acid).

Other types of polymers include a biodegradable, bioerodible polyester, such as poly(L-lactide) poly(D,L lactide), copolymers of lactide and glycolide such as poly(D,L-lactide-co-glycolide) and poly(L-lactide-co-glycolide), poly(caprolactone), poly(glycolide), copolymers prepared from caprolactone and/or lactide and/or glycolide and/or polyethylene glycol (e.g., copolymers of s-caprolactone and lactide and copolymers of glycolide and s-caprolactone), poly(valerolactone), polydioxanone, and copolymers of lactide and 1,4-dioxane-2-one. Other examples of biodegradable materials include poly(hydroxybutyrate), poly(hydroxyvalerate), poly(hydroxybutyrate-co-hydroxyvalerate) copolymers, poly(alkylcarbonate), poly(orthoesters), tyrosine based polycarbonates and polyarylates, poly(ethylene terephthalate), poly(anhydrides), poly(ester-amides), polyphosphazenes, or poly(amino acids).

In certain aspects, the devices of may comprise a non-degradable polymer. Representative examples of non-biodegradable polymers include ethylene-co-vinyl acetate copolymers, acrylic-based and methacrylic-based polymers (e.g., poly(acrylic acid), poly(methylacrylic acid), poly(methylmethacrylate), poly(hydroxyethylmethacrylate), poly(alkylcynoacrylate), poly(alkyl acrylates), poly(alkyl methacrylates)), poly(ethylene), poly(propylene), polyamides (e.g., nylon 6,6), poly(urethanes) (e.g., poly(ester urethanes), poly(ether urethanes), poly(carbonate urethanes), poly(ester-urea)), polyethers (e.g., poly(ethylene oxide)), poly(propylene oxide), poly(ethylene oxide)-poly(propylene oxide) copolymers, diblock and triblock copolymers, poly(tetramethylene glycol)], silicone containing polymers and vinyl-based polymers (e.g., polyvinylpyrrolidone, poly(vinyl alcohol), poly(vinyl acetate phthalate), and poly(styrene-co-isobutylene-co-styrene)). These compositions include copolymers as well as blends, crosslinked compositions and combinations of the above non-biodegradable polymers.

Perivascular devices can further comprise a matrix (e.g., polymeric carrier) to retain the compounds into or onto the device and to provide for sustained release of the compounds. In certain embodiments, device includes a matrix and a fibrous construct, where the fibrous construct serves to reinforce the matrix. In one aspect, the matrix is in the form of a coating. The matrix may contact all or only a portion of the fibrous construct and may reside only at the surface of the construct or may be impregnated into the material forming the fiber.

The matrix may be formulated from a variety of biodegradable and bioerodible polymers. The polymer matrix may include one or more biodegradable polymer(s), one or more non-degradable polymer(s) or a combination of one or more biodegradable polymer(s) and non-degradable polymer(s).

Representative examples of biodegradable polymers include naturally derived and synthetic biodegradable polymers.

Representative examples of naturally derived polymers include albumin, collagen, hyaluronic acid and derivatives, sodium alginate and derivatives, chitosan and derivatives gelatin, starch, cellulose polymers (for example methylcellulose, hydroxypropylcellulose, hydroxypropylmethylcellulose, carboxymethylcellulose, cellulose acetate phthalate, cellulose acetate succinate, hydroxypropylmethylcellulose phthalate), casein, dextran and derivatives, polysaccharides, and fibrinogen.

Representative examples of synthetic biodegradable polymers and copolymers include those formed from one or more cyclic monomers (e.g., D-lactide, L-lactide, D,L-lactide, glycolide, ε-caprolactone, trimethylene carbonate (TMC), p-dioxanone (e.g., 1,4-dioxane-2-one or 1,5-dioxepan-2-one), or a morpholinedione) and polymers and copolymers formed from one or more hydroxyl acids such as lactic acid or glycolic acid (e.g., poly(D,L-lactic acid) oligomers and polymers, poly(L-lactic acid) oligomers and polymers, poly(D-lactic acid) oligomers and polymers, poly(glycolic acid), poly(hydroxyvaleric acid), poly(malic acid), poly(tartronic acid), copolymers of lactic acid and ε-caprolactone, and copolymers of lactic acid and glycolic acid).

Other examples of biodegradable polymers for use in the matrix include include poly(hydroxybutyrate), poly(hydroxyvalerate), poly(hydroxybutyrate-co-hydroxyvalerate) copolymers, poly(alkylcarbonate), poly(orthoesters), tyrosine based polycarbonates and polyarylates, poly(ethylene terephthalate), poly(anhydrides), poly(ester-amides), polyphosphazenes, or poly(amino acids).

The matrix may comprise an amphiphilic polymer and may include two or more hydrophilic or hydrophobic blocks (e.g., a diblock (A-B) copolymer or a triblock (A-B-A) or (B-A-B) copolymer or a block copolymer of the form (AB)n-R or (BA)n-R where R is a multifunctional reagent (e.g. triethyl amine, pentaerythritol)).

The matrix may include a non-degradable polymer. Representative examples of non-biodegradable polymers include ethylene-co-vinyl acetate copolymers, acrylic-based and methacrylic-based polymers (e.g., poly(acrylic acid), poly(methylacrylic acid), poly(methylmethacrylate), poly(hydroxyethylmethacrylate), poly(alkylcynoacrylate), poly(alkyl acrylates), poly(alkyl methacrylates)), cellulose derivatives (e.g., cellulose esters and nitrocellulose) polyolefins such as poly(ethylene) and poly(propylene), polyamides (e.g., nylon 6,6), polyethers (e.g., poly(ethylene oxide), poly(propylene oxide), poly(ethylene oxide)-poly(propylene oxide) copolymers, and poly(tetramethylene glycol)), silicone containing polymers and vinyl-based polymers (polyvinylpyrrolidone, poly(vinyl alcohol), poly(vinyl acetate phthalate)), and poly(styrene-co-isobutylene-co-styrene). Other exemplary non-biodegradable polymers include poly(hydroxyethylmethacrylates) and poly(urethanes) (e.g., poly(ester urethanes), poly(ether urethanes), poly(carbonate urethanes), poly(ester-urea)). In certain embodiments, the compounds is delivered from a matrix (e.g., a film) made from a polyurethane or a styrene-isoprene-styrene copolymer. Commercially available aromatic and aliphatic polyurethanes which may be used, include, e.g., CHRONOFLEX AR, CHRONOFLEX AL, BIONATE, TECOFLEX, and the like. These compositions include copolymers as well as blends, crosslinked compositions and combinations of the above non-biodegradable polymers.

Exemplary materials for use in the practice of this disclosure are described in U.S. Pat. Nos. 6,575,887, and co-pending application, entitled “Perivascular Wraps,” filed Sep. 26, 2003 (U.S. Ser. No. 10/673,046); “Composite Drug Delivery System,” filed Sep. 15, 2006 (U.S. Ser. No. 60/844,814) and “Composite Drug Delivery System,” filed Nov. 22, 2006 (U.S. Ser. No. ______ not yet assigned), and in U.S. Pat. No. 6,534,693 and US Patent Application Nos. 2005/0281860; 2005/0084514; 2004/0071756; 2004/0018228 and 2004/0006296.

In certain aspects, the perivascular device may be made from a collagen. The device may be a drug-eluting collagen matrix or sleeve which (see, e.g., U.S. Pat. No. 6,726,923). This collagen matrix may be prefabricated, such as the BIOMEND (Sulzer Calciteck, Carlsbad, Calif.) or BIOPATCH (Ethicon, Somerville, N.J.) products and may contain other formulations, such as liposomes that may be loaded with bioactive agents and loaded into prefabricated collagen sheets.

The device may be a collagen tube-like collar such as TRINAM which is being developed by Ark Therapeutics (London, UK). The TRINAM technology as well as other related technology is described in, for example, (see, e.g., Fuster et al., Human Gene Therapy (2001) 12(16): 2025-2027) and US Patent Applications 2006/0093653 and 2003/0039694 and PCT Publication Nos. WO 99/55415 and WO 05/026206.

In other aspects, the perivascular device may be a drug-eluting, biodegradable tissue covering such as COLLAGRAN and COLACTIVE AG, denatured collagen-based matrices made up of three-dimensional scaffolds from Covalon (Canada) (see, e.g., U.S. Pat. Nos. 6,808,738; 6,475,516 and 6,228,393 and US Patent Application Publication Nos. 2006/0068013; 2002/0051812 and 2002/0009485).

Other materials composed of collagen or collagen and alginate, or chitosan or fibrin are described in, for example, U.S. Pat. No. 6,726,923 and US Patent Application Nos. 2005/0004158; 2004/0197409; and 2003/0113359.

Surgical materials, which may be combined with paclitaxel and dipyridamole (or analogues or derivatives thereof) according to the present disclosure, include commercially available products. Examples of materials into which the described compounds can be incorporated include INTERCEED (Johnson & Johnson, Inc.), PRECLUDE (W. L. Gore), and POLYACTIVE (poly(ether ester) multiblock copolymers (Osteotech, Inc., Shrewsbury, N.J.), based on poly(ethylene glycol) and poly(butylene terephthalate), and SURGICAL absorbable hemostat gauze-like sheet from Johnson & Johnson (New Brunswick, N.J.) which is an oxidized regenerated fibrillar cellulose hemostat agent. Another mesh is a prosthetic polypropylene mesh with a bioresorbable coating called SEPRAMESH Biosurgical Composite (Genzyme Corporation, Cambridge, Mass.). One side of the mesh is coated with a bioresorbable layer of sodium hyaluronate and carboxymethylcellulose, providing a temporary physical barrier that separates the underlying tissue and organ surfaces from the mesh. The other side of the mesh is uncoated, allowing for complete tissue ingrowth similar to bare polypropylene mesh. In one embodiment, the compounds may be applied only to the uncoated side of SEPRAMESH and not to the sodium hyaluronate/carboxymethylcellulose coated side. Other films and meshes include: (a) BARD MARLEX mesh (C.R. Bard, Inc.), which is a very dense knitted fabric structure with low porosity; (b) monofilament polypropylene mesh such as PROLENE available from Ethicon, Inc. Somerville, N.J. (see, e.g., U.S. Pat. Nos. 5,634,931 and 5,824,082)); (c) SURGISIS GOLD and SURGISIS IHM soft tissue graft (both from Cook Surgical, Inc.) which are devices specifically configured for use to reinforce soft tissue in repair of inguinal hernias in open and laparoscopic procedures; (d) thin walled polypropylene surgical meshes such as are available from Atrium Medical Corporation (Hudson, N.H.) under the trade names PROLITE, PROLITE ULTRA, and LITEMESH; (e) COMPOSIX hernia mesh (C.R. Bard, Murray Hill, N.J.), which incorporates a mesh patch (the patch includes two layers of an inert synthetic mesh, generally made of polypropylene, and is described in U.S. Pat. No. 6,280,453) that includes a filament to stiffen and maintain the device in a flat configuration; (f) VISILEX mesh (from C.R. Bard, Inc.), which is a polypropylene mesh that is constructed with monofilament polypropylene; (g) other meshes available from C.R. Bard, Inc. which include PERFIX Plug, KUGEL Hernia Patch, 3D MAX mesh, LHI mesh, DULEX mesh, and the VENTRALEX Hernia Patch; and (h) other types of polypropylene monofilament hernia mesh and plug products include HERTRA mesh 1, 2, and 2A, HERMESH 3, 4 & 5 and HERNIAMESH plugs T1, T2, and T3 from Herniamesh USA, Inc. (Great Neck, N.Y.).

Other examples of commercially available surgical meshes which may be combined with compounds are described below. One example includes a prosthetic polypropylene mesh with a bioresorbable coating sold under the trade name SEPRAMESH Biosurgical Composite (Genzyme Corporation). One side of the mesh is coated with a bioresorbable layer of sodium hyaluronate and carboxymethylcellulose, providing a temporary physical barrier that separates the underlying tissue and organ surfaces from the mesh. The other side of the mesh is uncoated, allowing for complete tissue ingrowth similar to bare polypropylene mesh. In one embodiment, the described compounds may be applied only to the uncoated side of SEPRAMESH and not to the sodium hyaluronate/carboxymethylcellulose coated side. Other examples of surgical sheets which can be used in the practice of this disclosure include those from Boston Scientific Corporation (TRELEX NATURAL Mesh, which is composed of a unique knitted polypropylene material); Ethicon, Inc. (knitted and woven VICRYL (polyglactin 910) meshes and MERSILENE Polyester Fiber Mesh); Dow Corning Corporation (Midland, Mich.), which sells a mesh material formed from silicone elastomer known as SILASTIC Rx Medical Grade Sheeting (Platinum Cured); United States Surgical/Syneture (Norwalk, Conn.) which sells a mesh made from absorbable polyglycolic acid under the trade name DEXON Mesh Products; Membrana Accurel Systems (Germany) which sells the CELGARD microporous polypropylene fiber and membrane; Gynecare Worldwide, a division of Ethicon, Inc. which sells a mesh material made from oxidized, regenerated cellulose known as INTERCEED TC7; Integra LifeSciences Corporation (Plainsboro, N.J.) which makes DURAGEN PLUS Adhesion Barrier Matrix.

The described perivascular materials may be applied to any bodily conduit or any tissue that may be prone to the development of fibrosis or intimal hyperplasia. Prior to implantation, the device may be trimmed or cut from a sheet of bulk material to match the configuration of the widened foramen, canal, or dissection region, or at a minimum, to overlay the exposed tissue area. The material may be bent or shaped to match the particular configuration of the placement region. The material may also be rolled in a cuff shape or cylindrical shape and placed around the exterior periphery of the desired tissue. The material may be an annular sheet with a cut end with or without slits. Slits provide a means of utilizing the wrap at a junction enabling more surface area of the wrap being in contact at the anastomotic site. This annular sheet is particularly well suited for being sutured around an aorta at a site of anastomosis with the sections between the slits being placed and sutured onto the graft (e.g., blood vessel or synthetic graft) that is joined to the aorta as described, for example, in US Patent Application No. 2003/0152609.

The perivascular delivery devices of this disclosure may be used for a variety of indications, including, without limitation, reduction of intimal hyperplasia and/or restenosis (e.g., resulting from insertion of vascular grafts or hemodialysis access devices) or in affiliation with devices and implants that lead to scarring as described herein (e.g., as a sleeve or mesh around a hemodialysis implant or vascular graft to reduce or inhibit scarring).

In one exemplary embodiment, the dipyridamole (or analogue or derivative) is coated on to (or into) the vascular graft as described herein, while the paclitaxel (or analogue or derivative) is administered via an adventitial wrap as described above.

Examples of conditions that may be treated or prevented with the described materials include iatrogenic complications of arterial and venous catheterization, complications of vascular dissection, complications of gastrointestinal passageway rupture and dissection, restonotic complications associated with vascular surgery (e.g., bypass surgery), and intimal hyperplasia.

In one aspect, the described compounds may be delivered from a material to the external walls of body passageways or cavities for the purpose of preventing and/or reducing a proliferative biological response that may obstruct or hinder the optimal functioning of the passageway or cavity, including, for example, iatrogenic complications of arterial and venous catheterization, aortic dissection, cardiac rupture, aneurysm, cardiac valve dehiscence, graft placement (e.g., A-V-bypass, peripheral bypass, CABG), fistula formation, passageway rupture and surgical wound repair.

Devices are described which may be used in the form of a perivascular wrap to prevent restenosis at anastomotic sites resulting from insertion of vascular grafts or hemodialysis access devices. In this case, perivascular wraps may be associated with or coated with the described compounds, which can be used in conjunction with a vascular graft to inhibit scarring at an anastomotic site. These devices may be placed or wrapped in a perivascular (periadventitial) manner around the outside of the anastomosis at the time of surgery. Implants comprising the described compounds may be used with synthetic bypass grafts (femoral-popliteal, femoral-femoral, axillary-femoral etc.), vein grafts (peripheral and coronary), internal mammary (coronary) grafts or hemodialysis grafts (AV fistulas, AV access grafts).

As perivascular devices are made in a variety of configurations and sizes, the exact dose of the administered compounds will vary with device size, surface area and design. Regardless of the method of application of the compounds to the device, the total amount (dose) of each compound in or on the device may be in the range of about 0.01 μg-10 μg, or 10 μg-10 mg, or 10 mg-250 mg, or 250 mg-1000 mg, or 1000 mg-2500 mg. The dose (amount) of each compound per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

In certain aspects, perivascular devices are provided that are associated with a combination of paclitaxel and dipyridamole, where the total amount of each compound on, in or near the device may be in an amount ranging from less than 0.01 μg to about 2500 μg per mm² of device surface area. Generally, the compound may be present in an amount ranging from less than 0.01 μg; or from 0.01 μg to about 1.0 μg; or from 0.01 μg to about 10 μg; or from about 0.5 μg to about 5 μg; or from about 0.05 μg to 50 μg; or from 10 μg to about 250 μg; or from 250 μg to about 2500 μg (per mm² of device surface area).

In certain embodiments, paclitaxel is present in an amount ranging from about 0.01 to about 1.0 μg/mm² and dipyridamole is present in an amount ranging from about 0.05 to about 50 μg/mm².

In other embodiments, paclitaxel is present in an amount ranging from about 0.1 to about 0.6 μg/mm² and dipyridamole is present in an amount ranging from about 0.5 to about 5 μg/mm².

The total amount of each compound made available on, in or near the device may be in an amount ranging from about 0.01 μg (micrograms) to about 2500 mg (milligrams).

Generally, the compounds may be in the amount ranging from 0.01 μg to about 10 μg; or from 10 μg to about 1 mg; or from 1 mg to about 10 mg; or from 10 mg to about 100 mg; or from 100 mg to about 500 mg; or from 500 mg to about 2500 mg.

In one embodiment, the weight ratio of dipyridamole to paclitaxel may exceed about 0.06 to about 1.0 to provide a superior biological effect. The weight ratio of dipyridamole to paclitaxel may be adjusted to exceed about 0.06; or about 0.08; or about 0.10; or about 0.20; or about 0.30 or about 0.40; or about 0.50; or about 0.60; or about 0.70; or about 0.80; or about 0.90; or about 1.0; or about 1.1; or about 1.2; or about 1.3; or about 1.4; or about 1.5; or about 1.6.

Soft Tissue Implants

In one aspect, the present disclosure provides for the combination of paclitaxel and dipyridamole (or analogues or derivatives thereof) and a soft tissue implant (e.g., breast implant, lip implant, facial implant, tissue filler, aesthetic implant and the like). Soft tissue implants that include a combination of compounds as described herein may be capable of inhibiting or reducing the overgrowth of granulation tissue, which can lead to encapsulation of the device, and may improve the clinical efficacy of these devices.

There are numerous types of soft tissue implants where the occurrence of a fibrotic reaction will adversely affect the functioning or appearance of the implant or the tissue surrounding the implant. Typically, fibrotic encapsulation of the soft tissue implant (or the growth of fibrous tissue between the implant and the surrounding tissue) can result in fibrous contracture and other problems that can lead to suboptimal appearance and patient comfort. Accordingly, the present disclosure provides for soft tissue implants that include a combination of compounds that are capable of inhibiting the formation of scar tissue to minimize or prevent encapsulation (and associated fibrous contracture) of the soft tissue implant.

Soft tissue implants are used in a variety of cosmetic, plastic, and reconstructive surgical procedures and may be delivered to many different parts of the body, including, without limitation, the face, nose, jaw, breast, chin, buttocks, chest, lip, and cheek. Soft tissue implants are used for the reconstruction of surgically or traumatically created tissue voids, augmentation of tissues or organs, contouring of tissues, the restoration of bulk to aging tissues, and to correct soft tissue folds or wrinkles (rhytides). Soft tissue implants may be used for the augmentation of tissue for cosmetic (aesthetic) enhancement or in association with reconstructive surgery following disease or surgical resection. Representative examples of soft tissue implants that can be coated with, or otherwise constructed to contain and/or release a combination of compounds provided herein, include, e.g., saline breast implants, silicone breast implants, triglyceride-filled breast implants, chin and mandibular implants, nasal implants, cheek implants, lip implants, and other facial implants, pectoral and chest implants, malar and submalar implants, and buttocks implants.

Specific examples of soft tissue implants and treatments which may be combined with a combination of compounds are described in greater detail below.

Breast Implants

In one aspect, the soft tissue implant is a breast implant. The breast implant may be placed for augmentation or breast reconstruction after mastectomy. In general, breast augmentation or reconstructive surgery involves the placement of a commercially available breast implant, which consists of a capsule filled with either saline or silicone, into the tissues underneath the mammary gland. Four different incision sites have historically been used for breast implantation: axillary (armpit), periareolar (around the underside of the nipple), inframamary (at the base of the breast where it meets the chest wall) and transumbilical (around the belly button). The tissue is dissected away through the small incision, often with the aid of an endoscope (particularly for axillary and transumbilical procedures where tunneling from the incision site to the breast is required). A pocket for placement of the breast implant is created in either the subglandular or the subpectorial region. For subglandular implants, the tissue is dissected to create a space between the glandular tissue and the pectoralis major muscle that extends down to the inframammary crease. For subpectoral implants, the fibres of the pectoralis major muscle are carefully dissected to create a space beneath the pectoralis major muscle and superficial to the rib cage. Careful hemostasis is essential (since it can contribute to complications such as capsular contractures), so much so that minimally invasive procedures (axillary, transumbilical approaches) must be converted to more open procedures (such as periareolar) if bleeding control is inadequate. Depending upon the type of surgical approach selected, the breast implant is often deflated and rolled up for placement in the patient. After accurate positioning is achieved, the implant can then be filled or expanded to the desired size.

A combination of compounds or composition delivered locally from the breast implant, administered locally into the tissue surrounding the breast implant, or administered systemically to reach the breast tissue, can minimize fibrous tissue formation, encapsulation and capsular contracture.

Incorporation of a a combination of compounds onto a breast implant (e.g., as a coating applied to the outer surface of the implant and/or incorporated into, and released from, the outer polymeric membrane of the implant) or into a breast implant (e.g., the agent is incorporated into the saline, gel or silicone within the implant and passively diffuses across the capsule into the surrounding tissue) may minimize or prevent fibrous contracture in response to gel or saline-containing breast implants that are placed subpectorally or subglandularly. Infiltration of a a combination of compounds or composition into the tissue surrounding the breast implant, or into the surgical pocket where the implant will be placed, is another strategy for preventing the formation of scar and capsular contracture in breast augmentation and reconstructive surgery. Each of these approaches for reducing complications arising from capsular contraction in breast implants is described herein.

Numerous breast implants are suitable for use in the practice of this disclosure and can be used for cosmetic and reconstructive purposes. Breast implants may be composed of a flexible soft shell filled with a fluid, such as saline solution, polysiloxane, or silicone gel. For example, the breast implant may be composed of an outer polymeric shell having a cavity filled with a plurality of hollow bodies of elastically deformable material containing a liquid saline solution. See, e.g., U.S. Pat. No. 6,099,565. The breast implant may be composed of an envelope of vulcanized silicone rubber that forms a hollow sealed water impermeable shell containing an aqueous solution of polyethylene glycol. See, e.g., U.S. Pat. No. 6,312,466. The breast implant may be composed of an envelope made from a flexible non-absorbable material and a filler material that is a shortening composition (e.g., vegetable oil). See, e.g., U.S. Pat. No. 6,156,066. The breast implant may be composed of a soft, flexible outer membrane and a partially-deformable elastic filler material that is supported by a compartmental internal structure. See, e.g., U.S. Pat. No. 5,961,552. The breast implant may be composed of a non-biodegradable conical shell filled with layers of monofilament yarns formed into resiliently compressible fabric. See, e.g., U.S. Pat. No. 6,432,138. The breast implant may be composed of a shell containing sterile continuous filler material made of continuous yarn of polyolefin or polypropylene. See, e.g., U.S. Pat. No. 6,544,287. The breast implant may be composed of an envelope containing a keratin hydrogel. See, e.g., U.S. Pat. No. 6,371,984. The breast implant may be composed of a hollow, collapsible shell formed from a flexible, stretchable material having a base portion reinforced with a resilient, non-deformable member and a cohesive filler material contained within. See, e.g., U.S. Pat. No. 5,104,409. The breast implant may be composed of a smooth, non-porous, polymeric outer envelope with an affixed non-woven, porous outer layer made of extruded fibers of polycarbonate urethane polymer, which has a soft filler material contained within. See, e.g., U.S. Pat. No. 5,376,117. The breast implant may be configured to be surgically implanted under the pectoral muscle with a second prosthesis implanted between the pectoral muscle and the breast tissue. See, e.g., U.S. Pat. No. 6,464,726. The breast implant may be composed of a homogenous silicone elastomer flexible shell of unitary construction with an interior filling and a rough-textured external surface with randomly formed interconnected cells to promote tissue ingrowth to prevent capsular contracture. See, e.g., U.S. Pat. No. 5,674,285. The breast implant may be a plastic implant with a covering of heparin, which is bonded to the surface to prevent or treat capsule formation and/or shrinkage in a blood dry tissue cavity. See, e.g., U.S. Pat. No. 4,713,073. The breast implant may be a sealed, elastic polymer envelope having a microporous structure that is filled with a viscoelastic material (e.g., salt of chondroitin sulfate) to provide a predetermined shape. See, e.g., U.S. Pat. No. 5,344,451.

Commercially available breast implant implants include those from INAMED Corporation (Santa Barbara, Calif.) that sells both Saline-Filled and Silicone-Filled Breast Implants. INAMED's Saline-Filled Breast Implants include the Style 68 Saline Matrix and Style 363LF as well as others in a variety of models, contours, shapes and sizes. INAMED's Silicone-Filled Breast Implants include the Style 10, Style 20 and Style 40 as well as others in a variety of shapes, contours and sizes. NAMED also sells breast tissue expanders, such as the NAMED Style 133 V series tissue expanders, which are used to encourage rapid tissue adherence to maximize expander immobility. Mentor Corporation (Santa Barbara, Calif.) sells the saline-filled Contour Profile Style Breast Implant (available in a variety of models, shapes, contours and sizes) and the SPECTRUM Postoperatively Adjustable Breast Implant that allows adjustment of breast size by adding or removing saline with a simple office procedure for six months post-surgery. Mentor also produces the Contour Profile® Gel (silicone) breast implant in a variety of models, shapes, contours and sizes. Breast implants such as these may benefit from release of a combination of compounds able to reduce scarring at the implant-tissue interface to minimize the incidence of fibrous contracture. In one aspect, the breast implant is combined with a a combination of compounds or composition containing a a combination of compounds. Ways that this can be accomplished include, but are not restricted to, incorporating a a combination of compounds into the polymer that composes the shell of the implant (e.g., the polymer that composes the capsule of the breast implant is loaded with an agent that is gradually released from the surface), surface-coating the breast implant with an a combination of compounds or a composition that includes an a combination of compounds, and/or incorporating the a combination of compounds into the implant filling material (for example, saline, gel, silicone) such that it can diffuse across the capsule into the surrounding tissue.

Facial and Aesthetic Implants

In one aspect, the soft tissue implant is a facial implant, including implants for the malar-midface region or submalar region (e.g., cheek implant). Malar and submalar augmentation is often conducted when obvious changes have occurred associated with aging (e.g., hollowing of the cheeks and ptosis of the midfacial soft tissue), midface hypoplasia (a dish-face deformity), post-traumatic and post-tumor resection deformities, and mild hemifacial microsomia. Malar and submalar augmentation may also be conducted for cosmetic purposes to provide a dramatic high and sharp cheek contour. Placement of a malar-submalar implant often enhances the result of a rhytidectomy or rhinoplasty by further improving facial balance and harmony.

There are numerous facial implants that can be used for cosmetic and reconstructive purposes. For example, the facial implant may be a thin teardrop-shaped profile with a broad head and a tapered narrow tail for the mid-facial or submalar region of the face to restore and soften the fullness of the cheeks. See, e.g., U.S. Pat. No. 4,969,901. The facial implant may be composed of a flexible material having a generally concave-curved lower surface and a convex-curved upper surface, which is used to augment the submalar region. See, e.g., U.S. Pat. No. 5,421,831. The facial implant may be a modular prosthesis composed of a thin planar shell and shims that provide the desired contour to the overlying tissue. See, e.g., U.S. Pat. No. 5,514,179. The facial implant may be composed of moldable silicone having a grid of horizontal and vertical grooves on a concave bone-facing rear surface to facilitate tissue ingrowth. See, e.g., U.S. Pat. No. 5,876,447. The facial implant may be composed of a closed-cell, cross-linked, polyethylene foam that is formed into a shell and of a shape to closely conform to the face of a human. See, e.g., U.S. Pat. No. 4,920,580. The facial implant may be a means of harvesting a dermis plug from the skin of the donor after applying a laser beam for ablating the epidermal layer of the skin thereby exposing the dermis and then inserting this dermis plug at a site of facial skin depression. See, e.g., U.S. Pat. No. 5,817,090. The facial implant may be composed of silicone-elastomer with an open-cell structure whereby the silicone elastomer is applied to the surface as a solid before the layer is cured. See, e.g., U.S. Pat. No. 5,007,929. The facial implant may be a hollow perforate mandibular or maxillary dental implant composed of a trans osseous bolt receptor that is secured against the alveolar ridge by contiguous straps. See, e.g., U.S. Pat. No. 4,828,492.

Commercially available facial implants suitable for the practice of this disclosure include: Tissue Technologies, Inc. (San Francisco, Calif.) sells the ULTRASOFT-RC Facial Implant which is made of soft, pliable synthetic e-PTFE used for soft tissue augmentation of the face. Tissue Technologies, Inc. also sells the ULTRASOFT, which is made of tubular e-PTFE indicated for soft tissue augmentation of the facial area and is particularly well suited for use in the lip border and the nasolabial folds. A variety of facial implants are available from ImplanTech Associates including the BINDER SUBMALAR facial implant, the BINDER SUBMALAR II FACIAL IMPLANT, the TERINO MALAR SHELL, the COMBINED SUBMALAR SHELL, the FLOWERS TEAR TROUGH implant; solid silicone facial and malar implants from Allied Biomedical; the Subcutaneous Augmentation Material (S.A.M.), made from microporous ePTFE which supports rapid tissue incorporation and preformed TRIMENSIONAL 3-D Implants from W. L. Gore & Associates, Inc. Juva Medical (Foster City, Calif.) has developed the FULFIL device for filling facial folds and augmentation of facial soft tissue, which is currently under FDA review. FULFIL consists of two components, an inflatable implant and a fill tube. The implant consists of a thin, outer membrane made from ePTFE. The inner surface of the ePTFE membrane is lined with a silicone elastomer. An integrated self-sealing silicone valve allows the device to be inflated with, and to retain, saline solution. The implant is pre-loaded onto the removable fill tube, which include a proximal female luer. The implant is positioned within the target tissue bed using standard surgical techniques and saline is injected into the implant via the fill tube. Once the appropriate amount of saline solution has been delivered into the implant to achieve the desired effect, the fill tube is withdrawn from the implant and suture reinforcement can be applied.

Chin and Mandibular Implants

In another aspect, the soft tissue implant is a chin or mandibular implant. Incorporation of a a combination of compounds into or onto the chin or mandibular implant, or infiltration of the agent into the tissue around a chin or mandibular implant, may minimize or prevent fibrous contracture in response to implants placed for cosmetic or reconstructive purposes.

Numerous chin and mandibular implants can be used for cosmetic and reconstructive purposes. For example, the chin implant may be a solid, crescent-shaped implant tapering bilaterally to form respective tails and having a curved projection surface positioned on the outer mandible surface to create a natural chin profile and form a build-up of the jaw. See, e.g., U.S. Pat. No. 4,344,191. The chin implant may be a solid crescent with an axis of symmetry of forty-five degrees, which has a softer, lower durometer material at the point of the chin to simulate the fat pad. See, e.g., U.S. Pat. No. 5,195,951. The chin implant may have a concave posterior surface to cooperate with the irregular bony surface of the mandible and a convex anterior surface with a protuberance for augmenting and providing a natural chin contour. See, e.g., U.S. Pat. No. 4,990,160. The chin implant may have a porous convex surface made of polytetrafluoroethylene having void spaces of size adequate to allow soft tissue ingrowth, while the concave surface made of silicone is nonporous to substantially preventing growth of bony tissue. See, e.g., U.S. Pat. No. 6,277,150.

Examples of commercially available chin or mandibular implants include: the TERINO EXTENDED ANATOMICAL chin implant, the GLASGOLD WAFER, the FLOWERS MANDIBULAR GLOVE, MITTELMAN PRE JOWL-CHIN, GLASGOLD WAFER implants, as well as other models from ImplantTech Associates; and the solid silicone chin implants from Allied Biomedical.

Nasal Implants

In another aspect, the soft tissue implant for use in the practice of this disclosure is a nasal implant. Incorporation of a combination of compounds into or onto the nasal implant, or infiltration of the agent into the tissue around a nasal implant, may minimize or prevent fibrous contracture in response to implants placed for cosmetic or reconstructive purposes.

Numerous nasal implants are suitable for the practice of this disclosure that can be used for cosmetic and reconstructive purposes. For example, the nasal implant may be elongated and contoured with a concave surface on a selected side to define a dorsal support end that is adapted to be positioned over the nasal dorsum to augment the frontal and profile views of the nose. See, e.g., U.S. Pat. No. 5,112,353. The nasal implant may be composed of substantially hard-grade silicone configured in the form of an hourglass with soft silicone at the tip. See, e.g., U.S. Pat. No. 5,030,232. The nasal implant may be composed of essentially a principal component being an aryl acrylic hydrophobic monomer with the remainder of the material being a cross-linking monomer and optionally one or more additional components selected from the group consisting of UV-light absorbing compounds and blue-light absorbing compounds. See, e.g., U.S. Pat. No. 6,528,602. The nasal implant may be composed of a hydrophilic synthetic cartilaginous material with pores of controlled size randomly distributed throughout the body for replacement of fibrous tissue. See, e.g., U.S. Pat. No. 4,912,141.

Examples of commercially available nasal implants suitable for use in the practice of this disclosure include the FLOWERS DORSAL, RIZZO DORSAL, SHIRAKABE, and DORSAL COLUMELLA nasal implants from ImplantTech Associates and solid silicone nasal implants from Allied Biomedical.

Lip Implants

In one aspect, the soft tissue implant suitable for combining with the compounds described herein is a lip implant. Incorporation of a combination of compounds into or onto the lip implant, or infiltration of the agent into the tissue around a lip implant, may minimize or prevent fibrous contracture in response to implants placed for cosmetic or reconstructive purposes.

Numerous lip implants can be used for cosmetic and reconstructive purposes. For example, the lip implant may be composed of non-biodegradable expanded, fibrillated polytetrafluoroethylene having an interior cavity extending longitudinally whereby fibrous tissue ingrowth may occur to provide soft tissue augmentation. See, e.g., U.S. Pat. Nos. 5,941,910 and 5,607,477. The lip implant may comprise soft, malleable, elastic, non-resorbing prosthetic particles that have a rough, irregular surface texture, which are dispersed in a non-retentive compatible physiological vehicle. See, e.g., U.S. Pat. No. 5,571,182.

Commercially available lip implants suitable for use in the present disclosure include SOFTFORM from Tissue Technologies, Inc. (San Francisco, Calif.), which has a tube-shaped design made of synthetic ePTFE; ALLODERM sheets (Allograft Dermal Matrix Grafts), which are sold by LifeCell Corporation (Branchburg, N.J.) may also be used as an implant to augment the lip. ALLODERM sheets are very soft and easily augment the lip in a diffuse manner. W. L. Gore and Associates (Newark, Del.) sells solid implantable threads that may also be used for lip implants.

Lip implants such as these may benefit from release of a combination of compounds able to reduce scarring at the implant-tissue interface to minimize the occurrence of fibrous contracture. Incorporation of a a combination of compounds into or onto a lip implant (e.g., as a coating applied to the surface, incorporated into the pores of a porous implant, incorporated into the implant, incorporated into the polymers that compose the outer capsule of the implant, incorporated into the threads or sheets that make up the lip implant and/or incorporated into the polymers that compose the inner portions of the implant) may minimize or prevent fibrous contracture in response to implants that are placed in the lips for cosmetic or reconstructive purposes. The a combination of compounds can reduce the incidence of asymmetry, skin dimpling, hardness and repeat interventions and improve patient satisfaction with the procedure. As an alternative to this, or in addition to this, a composition that includes an a combination of compounds can be injected or infiltrated into the lips directly.

Tissue Fillers

In one aspect, a combination of compounds as described herein may be combined with a composition for augmenting tissue (e.g., tissue filler). Soft tissue augmentation with tissue fillers has become a popular means of addressing contour defects that result from aging, photodamage, trauma, scarification, or disease. Injection of fillers usually requires the use of either a topical numbing cream or a local injection of numbing medication. The dermal filler is injected into each wrinkle or scar that requires treatment using a small needle. Incorporation of a combination of compounds into the tissue fillers, or infiltration of the agent locally into the tissue around the fillers or systemically to reach the site of injection may minimize or prevent fibrous contracture in response to fillers injected for cosmetic or reconstructive purposes.

Numerous tissue fillers to be used for cosmetic and reconstructive purposes are suitable for the practice of this disclosure. The fillers may be composed of bovine collagen, which may further be cross-linked. See, e.g., U.S. Pat. Nos. 4,488,911 and 4,582,640. The filler may be composed of human collagen, isolated for example, from harvested autologous tissue or from donor tissue. See, e.g., U.S. Pat. Nos. 5,332,802 and 6,743,435. The fillers may be composed of hyaluronic acid and may be further cross-linked. Hyaluronic acid can be isolated, for example, from animal sources or through bacterial fermentation. See, e.g., U.S. Pat. Nos. 4,885,244, 4,803,075, and 5,827,937. The fillers may be composed of synthetic materials, which can be formed into any one of numerous physical shapes, such as microspheres. Synthetic fillers may be further combined with collagen or hyaluronic acid fillers. See, e.g., U.S. Pat. Nos. 5,344,452, 6,432,437, and 6,716,251.

Commercially available tissue fillers include those manufactured by INAMED Corporation (Santa Barbara, Calif.), such as the collagen based fillers ZYDERM, composed of purified fibriller collagen isolated from isolated herds of domestic cattle, ZYPLAST, composed of bovine dermal collagen cross-linked by glutaraldehyde, and COSMODERM and COSMOPLAST, composed of human collagen grown under controlled laboratory conditions that is not cross-linked or cross-linked with glutaraldehyde, respectively. Collagen Matrix Technologies and Angiotech Incorporated manufacture REFILLE, a filler based on collagen matrices derived from donated human dermis that also contains matrix proteins, such as elastin. Hyaluronic acid based fillers include HYLAFORM GEL, a form of cross-linked hyaluronic acid derived from rooster combs of domestic fowl (manufactured by INAMED), RESTYLANE, derived from streptococcal bacterial fermentation (manufactured by Medicis), and JUVADERM, also obtained from bacterial fermentation (manufactured by INAMED). Fillers incorporating synthetic materials include ARTEFILL, composed of polymethacrylate microspheres suspended in bovine collagen (manufactured by Artes Medical), RADIESSE, composed of calcium hydroxyapatite microspheres suspended in an aqueous gel carrier (manufactured by Bioform), and SCULPTURA, composed of poly-L-lactic acid microspheres (manufactured by Dermik Aesthetics).

As soft tissue implants are made in a variety of configurations sizes and include a variety of different materials, the exact dose of the administered compounds will vary with device size, composition, surface area and design. Regardless of the method of application of the compounds to the device, the total amount (dose) of each compound in or on the device may be in the range of about 0.01 μg-10 μg, or 10 μg-10 mg, or 10 mg-250 mg, or 250 mg-1000 mg, or 1000 mg-2500 mg. The dose (amount) of each compound per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

In certain aspects, soft tissue implants are provided that are associated with a combination of paclitaxel and dipyridamole, where the total amount of each compound on, in or near the device may be in an amount ranging from less than 0.01 μg to about 2500 μg per mm² of device surface area. Generally, the compound may be present in an amount ranging from less than 0.01 μg; or from 0.01 μg to about 1.0 μg; or from 0.01 μg to about 10 μg; or from about 0.5 μg to about 5 μg; or from about 0.05 μg to 50 μg; or from 10 μg to about 250 μg; or from 250 μg to about 2500 μg (per mm² of device surface area).

In certain embodiments, paclitaxel is present in an amount ranging from about 0.01 to about 1.0 μg/mm² and dipyridamole is present in an amount ranging from about 0.05 to about 50 μg/mm².

In other embodiments, paclitaxel is present in an amount ranging from about 0.1 to about 0.6 μg/mm² and dipyridamole is present in an amount ranging from about 0.5 to about 5 μg/mm².

The total amount of each compound made available on, in or near the device may be in an amount ranging from about 0.01 μg (micrograms) to about 2500 mg (milligrams).

Generally, the compounds may be in the amount ranging from 0.01 μg to about 10 μg; or from 10 μg to about 1 mg; or from 1 mg to about 10 mg; or from 10 mg to about 100 mg; or from 100 mg to about 500 mg; or from 500 mg to about 2500 mg.

In one embodiment, the weight ratio of dipyridamole to paclitaxel may exceed about 0.06 to about 1.0 to provide a superior biological effect. The weight ratio of dipyridamole to paclitaxel may be adjusted to exceed about 0.06; or about 0.08; or about 0.10; or about 0.20; or about 0.30 or about 0.40; or about 0.50; or about 0.60; or about 0.70; or about 0.80; or about 0.90; or about 1.0; or about 1.1; or about 1.2; or about 1.3; or about 1.4; or about 1.5; or about 1.6.

Intraocular Implants

In another aspect, the present disclosure provides for a combination of compounds and an intraocular implant.

In one embodiment, the intraocular implant is an intraocular lens device for the prevention of lens (e.g., anterior or posterior lens) opacification. Eyesight deficiencies that may be treated with intraocular lenses include, without limitation, cataracts, myopia, hyperopia, astigmatism and other eye diseases. Intraocular lenses are most commonly used to replace the natural crystalline lens which is removed during cataract surgery. A cataract results from a change in the transparency of the normal crystalline lens in the eye. When the lens becomes opaque from calcification (e.g., yellow and/or cloudy), the light cannot enter the eye properly and vision is impaired.

Implantation of intraocular lenses into the eye is a standard technique to restore useful vision in diseased or damaged eyes. The number of intraocular lenses implanted in the United States has grown exponentially over the last decade. Currently, over 1 million intraocular lenses are implanted annually, with the vast majority (90%) being placed in the posterior chamber of the eye. The intent of intraocular lenses is to replace the natural crystalline lens (i.e., aphakic eye) or to supplement and correct refractive errors (i.e., phakic eye, natural crystalline lens is not removed).

Implanted intraocular lenses may develop complications caused by mechanical trauma, inflammation, infection or optical problems. Mechanical and inflammatory injury may lead to reduced vision, chronic pain, secondary cataracts, corneal decompensation, cystoid macular edema, hyphema, uveitis or glaucoma. One common problem that occurs with cataract extraction is opacification which results from the tissue's reaction to the surgical procedure or to the artificial lens. Opacification leads to clouding of the intraocular lens, thus reducing the long-term benefits. Opacification typically results when proliferation and migration of epithelial cells occur along the posterior capsule behind the intraocular lens. Subsequent surgery may be required to correct this reaction; however, it involves a complex technical process and may lead to further serious, sight-threatening complications. Therefore, coating or incorporating the intraocular lens with a combination of compounds as described herein may reduce these complications.

Representative examples of intraocular lenses that can benefit from being coated with or having incorporated therein a a combination of compounds include, without limitation, polymethylmethacrylate (PMMA) intraocular lenses, silicone intraocular lenses, achromatic lenses, pseudophakos, phakic lenses, aphakic lenses, multi-focal intraocular lenses, hydrophilic and hydrophobic acrylic intraocular lenses, intraocular implants, optic lenses and rigid gas permeable (RGP) lenses.

In one aspect, the intraocular lens may be used as an implant for the treatment of cataracts, where the natural crystalline lens of the eye has been removed (i.e., aphakic lens).

In another aspect, the intraocular lens may be used as a corrective implant for vision impairment, where the natural crystalline lens of the eye has not been removed (i.e., phakic lens).

In another aspect, the intraocular lens may be a multi-focal lens capable of variable accommodation to enable the user to look through different portions of the lens to achieve different levels of focusing power.

Intraocular lenses, which may be combined with one or more agents according to the present disclosure, include commercially available products. For example, Alcon Laboratories, Inc. (Fort Worth, Tex.) sells the foldable ACRYSOF Intraocular Lens. Bausch & Lomb Surgical, Inc. (San Dimas, Calif.) sells the foldable SOFLEX SE Intraocular Lens. Advanced Medical Optics, Inc (Santa Ana, Calif.) sells the CLARIFLEX Foldable Intraocular Lens, SENSAR Acrylic Intraocular Lens, and PHACOFLEX II SI40NB and SI30NB.

In another aspect, the intraocular implant may be a spacer designed to be inserted into surgical incisions made in the sclera of an individual suffering from presbyopia. Presyopia is the eye's diminished power of accommodation that occurs with aging. Presbyopia is not a disease as such, but a condition that affects everyone at a certain age. The first symptoms are usually noticed between the ages of 40-50. Surgical correction of presbyopia involves making four small radial incisions in each quadrant of the sclera. In order to prevent contraction of the scleral incisions, tissue barriers, or spacers, made of an inert substance are inserted into the incisions and secured by suture. The NUFOCUS spacers developed by Hays and Thornton and being manufactured by Angiotech Inc. are formed from medical grade silicone have an elongate bar shape, measuring 2.5 mm in length and 0.6 mm in width and are secured with 10-0 blue polypropylene sutures.

The intraocular implant may comprise a combination of compounds or a composition that includes the compounds directly. Alternatively, or in addition, the compounds may be coated, absorbed into, or bound onto the lens or implant surface (e.g., to the haptics), or may be released from a hole (pore) or cavity outside the optical part of the lens or on the implant surface. Alternatively or in addition, the compounds may be coated, absorbed into, or bound onto the surface of a suture used to secure an implant during surgery.

The intraocular implants of this disclosure may be used in various surgical procedures. For example, the intraocular implant may be used in conjunction with a transplant for the cornea. Synthetic corneas can be used in patients loosing vision due to a degenerative cornea. Implanted synthetic corneas can restore patient vision, however, they often induce a fibrous foreign body response that limits their use. The intraocular implant of the present disclosure can prevent the foreign body response to the synthetic cornea and extend the cornea longevity. In another example, the synthetic cornea itself is coated with the agents of this disclosure, thus minimizing tissue reaction to corneal implantation.

In another aspect, the intraocular lens or implant may be used in conjunction with treatment of secondary cataract after extracapsular cataract extraction.

As intraocular implants are made in a variety of configurations sizes and include a variety of different materials, the exact dose of the administered compounds will vary with device size, composition, surface area and design. Regardless of the method of application of the compounds to the device, the total amount (dose) of each compound in or on the device may be in the range of about 0.01 μg-10 μg, or 10 μg-10 mg, or 10 mg-250 mg, or 250 mg-1000 mg, or 1000 mg-2500 mg. The dose (amount) of each compound per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

In certain aspects, intraocular implants are provided that are associated with a combination of paclitaxel and dipyridamole, where the total amount of each compound on, in or near the device may be in an amount ranging from less than 0.01 μg to about 2500 μg per mm² of device surface area. Generally, the compound(s) may be present in an amount ranging from less than 0.01 μg; or from 0.01 μg to about 1.0 μg; or from 0.01 μg to about 10 μg; or from about 0.5 μg to about 5 μg; or from about 0.05 μg to 50 μg; or from 10 μg to about 250 μg; or from 250 μg to about 2500 μg (per mm² of device surface area).

In certain embodiments, paclitaxel is present in an amount ranging from about 0.01 to about 1.0 μg/mm² and dipyridamole is present in an amount ranging from about 0.05 to about 50 μg/mm².

In other embodiments, paclitaxel is present in an amount ranging from about 0.1 to about 0.6 μg/mm² and dipyridamole is present in an amount ranging from about 0.5 to about 5 μg/mm².

The total amount of each compound made available on, in or near the device may be in an amount ranging from about 0.01 μg (micrograms) to about 2500 mg (milligrams). Generally, the compounds may be in the amount ranging from 0.01 mg to about 10 μg; or from 10 μg to about 1 mg; or from 1 mg to about 10 mg; or from 10 mg to about 100 mg; or from 100 mg to about 500 mg; or from 500 mg to about 2500 mg.

In one embodiment, the weight ratio of dipyridamole to paclitaxel may exceed about 0.06 to about 1.0 to provide a superior biological effect. The weight ratio of dipyridamole to paclitaxel may be adjusted to exceed about 0.06; or about 0.08; or about 0.10; or about 0.20; or about 0.30 or about 0.40; or about 0.50; or about 0.60; or about 0.70; or about 0.80; or about 0.90; or about 1.0; or about 1.1; or about 1.2; or about 1.3; or about 1.4; or about 1.5; or about 1.6.

Electrical Devices

In one aspect, the present disclosure provides for the combination of paclitaxel and dipyridamole (or analogues or derivatives thereof) and an electrical device

“Electrical device” refers to a medical device having electrical components that can be placed in contact with tissue in an animal host and can provide electrical excitation to nervous or muscular tissue. Electrical devices can generate electrical impulses and may be used to treat many bodily dysfunctions and disorders by blocking, masking, or stimulating electrical signals within the body. Electrical medical devices of particular utility in the present disclosure include, but are not restricted to, devices used in the treatment of cardiac rhythm abnormalities, pain relief, epilepsy, Parkinson's Disease, movement disorders, obesity, depression, anxiety and hearing loss. Examples of electrical devices include neurostimulators, cardiac stimulation devices, and electrical leads.

“Neurostimulator” or “Neurostimulation Device” refers to an electrical device for electrical excitation of the central, autonomic, or peripheral nervous system. The neurostimulator sends electrical impulses to an organ or tissue. The neurostimulator may include electrical leads as part of the electrical stimulation system. Neurostimulation may be used to block, mask, or stimulate electrical signals in the body to treat dysfunctions, including, without limitation, pain, seizures, anxiety disorders, depression, ulcers, deep vein thrombosis, muscular atrophy, obesity, joint stiffness, muscle spasms, osteoporosis, scoliosis, spinal disc degeneration, spinal cord injury, deafness, urinary dysfunction and gastroparesis. Neurostimulation may be delivered to many different parts of the nervous system, including, spinal cord, brain, vagus nerve, sacral nerve, gastric nerve, auditory nerves, as well as organs, bone, muscles and tissues. As such, neurostimulators are developed to conform to the different anatomical structures and nervous system characteristics.

“Cardiac Stimulation Device” or “Cardiac Rhythm Management Device” or “Cardiac Pacemaker” or “Implantable Cardiac Defibrillator (ICD)” all refer to an electrical device for electrical excitation of cardiac muscle tissue (including the specialized cardiac muscle cells that make up the conductive pathways of the heart). The cardiac pacemaker sends electrical impulses to the muscle (myocardium) or conduction tissue of the heart. The pacemaker may include electrical leads as part of the electrical stimulation system. Cardiac pacemakers may be used to block, mask, or stimulate electrical signals in the heart to treat dysfunctions, including, without limitation, atrial rhythm abnormalities, conduction abnormalities and ventricular rhythm abnormalities.

“Electrical lead” refers to an electrical device that is used as a conductor to carry electrical signals from the generator to the tissues. Typically, electrical leads are composed of a connector assembly, a lead body (i.e., conductor) and an electrode. The electrical lead may be a wire or other material that transmits electrical impulses from a generator (e.g., pacemaker, defibrillator, or other neurostimulator). Electrical leads may be unipolar, in which they are adapted to provide effective therapy with only one electrode. Multi-polar leads are also available, including bipolar, tripolar and quadripolar leads.

Medical devices having electrical components, such as electrical pacing or stimulating devices, can be implanted in the body to provide electrical conduction to the central and peripheral nervous system (including the autonomic system), cardiac muscle tissue (including myocardial conduction pathways), smooth muscle tissue and skeletal muscle tissue. These electrical impulses are used to treat many bodily dysfunctions and disorders by blocking, masking, stimulating, or replacing electrical signals within the body. Examples include pacemaker leads used to maintain the normal rhythmic beating of the heart; defibrillator leads used to “re-start” the heart when it stops beating; peripheral nerve stimulating devices to treat chronic pain; deep brain electrical stimulation to treat conditions such as tremor, Parkinson's disease, movement disorders, epilepsy, depression and psychiatric disorders; and vagal nerve stimulation to treat epilepsy, depression, anxiety, obesity, migraine and Alzheimer's Disease.

The clinical function of an electrical device such as a cardiac pacemaker lead, neurostimulation lead, or other electrical lead depends upon the device being able to effectively maintain intimate anatomical contact with the target tissue (typically electrically excitable cells such as muscle or nerve) such that electrical conduction from the device to the tissue can occur. Unfortunately, in many instances when these devices are implanted in the body, they are subject to a “foreign body” response from the surrounding host tissues. The body recognizes the implanted device as foreign, which triggers an inflammatory response followed by encapsulation of the implant with fibrous connective tissue (or glial tissue called “gliosis”—when it occurs within the central nervous system). Scarring (i.e., fibrosis or gliosis) can also result from trauma to the anatomical structures and tissue surrounding the implant during the implantation of the device. Lastly, fibrous encapsulation of the device can occur even after a successful implantation if the device is manipulated (some patients continuously “fiddle” with a subcutaneous implant) or irritated by the daily activities of the patient. When scarring occurs around the implanted device, the electrical characteristics of the electrode-tissue interface degrade, and the device may fail to function properly. For example, it may require additional electrical current from the lead to overcome the extra resistance imposed by the intervening scar (or glial) tissue. This can shorten the battery life of an implant (making more frequent removal and re-implantation necessary), prevent electrical conduction altogether (rendering the implant clinically ineffective) and/or cause damage to the target tissue. Additionally, the surrounding tissue may be inadvertently damaged from the inflammatory foreign body response, which can result in loss of function or tissue necrosis.

Neurostimulation Devices

In one aspect, the electrical device may be a neurostimulation device where a pulse generator delivers an electrical impulse to a nervous tissue (e.g., CNS, peripheral nerves, autonomic nerves) in order to regulate its activity. There are numerous neurostimulator devices where the occurrence of a fibrotic reaction may adversely affect the functioning of the device or the biological problem for which the device was implanted or used. Typically, fibrotic encapsulation of the electrical lead (or the growth of fibrous tissue between the lead and the target nerve tissue) slows, impairs, or interrupts electrical transmission of the impulse from the device to the tissue. This can cause the device to function suboptimally or not at all, or can cause excessive drain on battery life because increased energy is required to overcome the electrical resistance imposed by the intervening scar (or glial) tissue.

Neurostimulation devices are used as alternative or adjunctive therapy for chronic, neurodegenerative diseases, which are typically treated with drug therapy, invasive therapy, or behavioral/lifestyle changes. Neurostimulation may be used to block, mask, or stimulate electrical signals in the body to treat dysfunctions, including, without limitation, pain, seizures, anxiety disorders, depression, ulcers, deep vein thrombosis, muscular atrophy, obesity, joint stiffness, muscle spasms, osteoporosis, scoliosis, spinal disc degeneration, spinal cord injury, deafness, urinary dysfunction and gastroparesis. Neurostimulation may be delivered to many different parts of the nervous system, including, spinal cord, brain, vagus nerve, sacral nerve, gastric nerve, auditory nerves, as well as organs, bone, muscles and tissues. As such, neurostimulators are developed to conform to the different anatomical structures and nervous system characteristics. Representative examples of neurologic and neurosurgical implants and devices that can be coated with, or otherwise constructed to contain and/or release the compounds provided herein, include, e.g., nerve stimulator devices to provide pain relief, devices for continuous subarachnoid infusions, implantable electrodes, stimulation electrodes, implantable pulse generators, electrical leads, stimulation catheter leads, neurostimulation systems, electrical stimulators, cochlear implants, auditory stimulators and micro stimulators.

In separate aspects, the following exemplary neurostimulation devices that may be combined with paclitaxel and dipyridamole include neurostimulation devices for the treatment of chronic pain, the treatment of Parkinson's Disease; vagal nerve stimulation for the treatment of epilepsy and other disorders; sacral nerve stimulation for bladder control problems; gastric nerve stimulation for the treatment of GI disorders; cochlear implants for the treatment of deafness; and electrical stimulation to promote bone growth.

Examples of commercially available neurostimulation products that may be associated with a combination of compounds as described herein include the radio-frequency powered neurostimulator comprised of the 3272 MATTRIX Receiver, 3210 MATTRIX Transmitter and 3487A PISCES-QUAD Quadripolar Leads made by Medtronic, Inc. (Minneapolis, Minn.). Medtronic also sells a battery-powered ITREL 3 Neurostimulator and SYNERGY Neurostimulator, the INTERSIM Therapy for sacral nerve stimulation for urinary control, and leads such as the 3998 SPECIFY Lead and 3587A RESUME II Lead. Another example of a neurostimulation device is a gastric pacemaker, in which multiple electrodes are positioned along the GI tract to deliver a phased electrical stimulation to pace peristaltic movement of the material through the GI tract. See, e.g., U.S. Pat. No. 5,690,691. A representative example of a gastric stimulation device is the ENTERRA Gastric Electrical Stimulation (GES) from Medtronic, Inc. (Minneapolis, Minn.).

Cardiac Rhythm Management (CRM) Devices

In another aspect, the electrical device may be a cardiac pacemaker device where a pulse generator delivers an electrical impulse to myocardial tissue (often specialized conduction fibres) via an implanted lead in order to regulate cardiac rhythm. Typically, electrical leads are composed of a connector assembly, a lead body (i.e., conductor) and an electrode. Representative examples of electrical leads include, without limitation, medical leads, cardiac leads, pacer leads, pacing leads, pacemaker leads, endocardial leads, endocardial pacing leads, cardioversion/defibrillator leads, cardioversion leads, epicardial leads, epicardial defibrillator leads, patch defibrillators, patch leads, electrical patch, transvenous leads, active fixation leads, passive fixation leads and sensing leads. Representative examples of CRM devices that utilize electrical leads include: pacemakers, LVAD's, defibrillators, implantable sensors and other electrical cardiac stimulation devices.

There are numerous pacemaker devices where the occurrence of a fibrotic reaction will adversely affect the functioning of the device or cause damage to the myocardial tissue. Typically, fibrotic encapsulation of the pacemaker lead (or the growth of fibrous tissue between the lead and the target myocardial tissue) slows, impairs, or interrupts electrical transmission of the impulse from the device to the myocardium. For example, fibrosis is often found at the electrode-myocardial interfaces in the heart, which may be attributed to electrical injury from focal points on the electrical lead. The fibrotic injury may extend into the tricuspid valve, which may lead to perforation. Fibrosis may lead to thrombosis of the subclavian vein; a condition which may be life-threatening. Electrical leads that release compounds for reducing scarring at the electrode-tissue interface may help prolong the clinical performance of these devices. Not only can fibrosis cause the device to function suboptimally or not at all, it can cause excessive drain on battery life as increased energy is required to overcome the electrical resistance imposed by the intervening scar tissue. Similarly, fibrotic encapsulation of the sensing components of a rate-responsive pacemaker (described below) can impair the ability of the pacemaker to identify and correct rhythm abnormalities leading to inappropriate pacing of the heart or the failure to function correctly when required.

Several different electrical pacing devices are used in the treatment of various cardiac rhythm abnormalities including pacemakers, implantable cardioverter defibrillators (ICD), left ventricular assist devices (LVAD), and vagus nerve stimulators (stimulates the fibers of the vagus nerve which in turn innervate the heart). The pulse generating portion of device sends electrical impulses via implanted leads to the muscle (myocardium) or conduction tissue of the heart to affect cardiac rhythm or contraction. Pacing can be directed to one or more chambers of the heart. Cardiac pacemakers may be used to block, mask, or stimulate electrical signals in the heart to treat dysfunctions, including, without limitation, atrial rhythm abnormalities, conduction abnormalities and ventricular rhythm abnormalities. ICDs are used to depolarize the ventricals and re-establish rhythm if a ventricular arrhythmia occurs (such as asystole or ventricular tachycardia) and LVADs are used to assist ventricular contraction in a failing heart.

Cardiac rhythm devices, and in particular the lead(s) that deliver the electrical pulsation, must be positioned in a very precise manner to ensure that stimulation is delivered to the correct anatomical location in the heart. All, or parts, of a pacing device can migrate following surgery, or excessive scar tissue growth can occur around the lead, which can lead to a reduction in the performance of these devices (as described previously). Cardiac rhythm management devices that release a compounds for reducing scarring at the electrode-tissue interface can be used to increase the efficacy and/or the duration of activity (particularly for fully-implanted, battery-powered devices) of the implant. Accordingly, the present disclosure provides cardiac leads that are associated with a combination of compounds or a composition that includes a combination of compounds.

Commercially available pacemakers suitable for the practice of this disclosure include the KAPPA SR 400 Series single-chamber rate-responsive pacemaker system, the KAPPA DR 400 Series dual-chamber rate-responsive pacemaker system, the KAPPA 900 and 700 Series single-chamber rate-responsive pacemaker system, and the KAPPA 900 and 700 Series dual-chamber rate-responsive pacemaker system by Medtronic, Inc. Medtronic pacemaker systems utilize a variety leads including the CAPSURE Z Novus, CAPSUREFIX Novus, CAPSUREFIX, CAPSURE SP Novus, CAPSURE SP, CAPSURE EPI and the CAP SURE VDD which may be suitable for coating with a a combination of compounds. Pacemaker systems and associated leads that are made by Medtronic are described in, e.g., U.S. Pat. Nos. 6,741,893; 5,480,441; 5,411,545; 5,324,310; 5,265,602; 5,265,601; 5,241,957 and 5,222,506. Medtronic also makes a variety of steroid-eluting leads including those described in, e.g., U.S. Pat. Nos. 5,987,746; 6,363,287; 5,800,470; 5,489,294; 5,282,844 and 5,092,332. The INSIGNIA single-chamber and dual-chamber system, PULSAR MAX II DR dual-chamber adaptive-rate pacemaker, PULSAR MAX II SR single-chamber adaptive-rate pacemaker, DISCOVERY II DR dual-chamber adaptive-rate pacemaker, DISCOVERY II SR single-chamber adaptive-rate pacemaker, DISCOVERY II DDD dual-chamber pacemaker, and the DISCOVERY II SSI dingle-chamber pacemaker systems made by Guidant Corp. (Indianapolis, Ind.) are also suitable pacemaker systems for the practice of this disclosure. Once again, the leads from the Guidant pacemaker systems may be suitable for coating with a combination of compounds. Pacemaker systems and associated leads that are made by Guidant are described in, e.g., U.S. Pat. Nos. 6,473,648; 6,345,204; 6,321,122; 6,152,954; 5,769,881; 5,284,136; 5,086,773 and 5,036,849. The AFFINITY DR, AFFINITY VDR, AFFINITY SR, AFFINITY DC, ENTITY, IDENTITY, IDENTITY ADX, INTEGRITY, INTEGRITY DR, INTEGRITY ADx, MICRONY, REGENCY, TRILOGY, and VERITY ADx, pacemaker systems and leads from St. Jude Medical, Inc. (St. Paul, Minn.) may also be suitable for use with a fibrosis-inhibiting coating to improve electrical transmission and sensing by the pacemaker leads. Pacemaker systems and associated leads that are made by St. Jude Medical are described in, e.g., U.S. Pat. Nos. 6,763,266; 6,760,619; 6,535,762; 6,246,909; 6,198,973; 6,183,305; 5,800,468 and 5,716,390. Alternatively, the combination of compounds may be infiltrated into the region around the electrode-cardiac muscle interface under the present disclosure. It should be obvious to one of skill in the art that commercial pacemakers not specifically sited as well as next-generation and/or subsequently developed commercial pacemaker products are to be anticipated and are suitable for use under the present disclosure.

Other types of devices which may be associated with the combination of compounds described herein include implantable cardioverter defibrillator (ICD) systems, vagus nerve stimulation devices for the treatment of arrhythmia, and neurostimulation devices that may be used to stimulate the vagus nerve and affect the rhythm of the heart.

As electrical devices (e.g., neurostimulators, CRM devices, leads, electrodes, and the like) are made in a variety of configurations and sizes, the exact dose of the administered compounds will vary with device size, surface area and design. Regardless of the method of application of the compounds to the device, the total amount (dose) of each compound in or on the device may be in the range of about 0.01 μg-10 μg, or 10 μg-10 mg, or 10 mg-250 mg, or 250 mg-1000 mg, or 1000 mg-2500 mg. The dose (amount) of each compound per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

In certain aspects, electrical devices are provided that are associated with a combination of paclitaxel and dipyridamole, where the total amount of each compound on, in or near the device may be in an amount ranging from less than 0.01 μg to about 2500 μg per mm² of device surface area. Generally, the compound(s) may be present in an amount ranging from less than 0.01 μg; or from 0.01 μg to about 1.0 μg; or from 0.01 μg to about 10 μg; or from about 0.5 μg to about 5 μg; or from about 0.05 μg to 50 μg; or from 10 μg to about 250 μg; or from 250 μg to about 2500 μg (per mm² of device surface area).

In certain embodiments, paclitaxel is present in an amount ranging from about 0.01 to about 1.0 μg/mm² and dipyridamole is present in an amount ranging from about 0.05 to about 50 μg/mm².

In other embodiments, paclitaxel is present in an amount ranging from about 0.1 to about 0.6 μg/mm² and dipyridamole is present in an amount ranging from about 0.5 to about 5 μg/mm².

The total amount of each compound made available on, in or near the device may be in an amount ranging from about 0.01 μg (micrograms) to about 2500 mg (milligrams). Generally, the compounds may be in the amount ranging from 0.01 μg to about 10 μg; or from 10 μg to about 1 mg; or from 1 mg to about 10 mg; or from 10 mg to about 100 mg; or from 100 mg to about 500 mg; or from 500 mg to about 2500 mg.

In one embodiment, the weight ratio of dipyridamole to paclitaxel may exceed about 0.06 to about 1.0 to provide a superior biological effect. The weight ratio of dipyridamole to paclitaxel may be adjusted to exceed about 0.06; or about 0.08; or about 0.10; or about 0.20; or about 0.30 or about 0.40; or about 0.50; or about 0.60; or about 0.70; or about 0.80; or about 0.90; or about 1.0; or about 1.1; or about 1.2; or about 1.3; or about 1.4; or about 1.5; or about 1.6.

Adhesion Barriers

In another aspect, devices are provided for reducing or prevent the formation of adhesions that occur between tissues following surgery, injury or disease. In certain aspects, the devices may be in the form of films and meshes that include a combination of compounds (e.g., paclitaxel and dipyridamole) for use as surgical adhesion barriers. Adhesion formation, a complex process in which bodily tissues that are normally separate grow together, occurs most commonly as a result of surgical intervention and/or trauma. Generally, adhesion formation is an inflammatory reaction in which factors are released, increasing vascular permeability and resulting in fibrinogen influx and fibrin deposition. This deposition forms a matrix that bridges the abutting tissues. Fibroblasts accumulate, attach to the matrix, deposit collagen and induce angiogenesis. If this cascade of events can be prevented within 4 to 5 days following surgery, then adhesion formation can be inhibited. Adhesion formation or unwanted scar tissue accumulation and encapsulation complicates a variety of surgical procedures and virtually any open or endoscopic surgical procedure in the abdominal or pelvic cavity. Encapsulation of surgical implants also complicates breast reconstruction surgery, joint replacement surgery, hernia repair surgery, artificial vascular graft surgery, and neurosurgery. In each case, the implant becomes encapsulated by a fibrous connective tissue capsule which compromises or impairs the function of the surgical implant (e.g., breast implant, artificial joint, surgical mesh, vascular graft, dural patch). Chronic inflammation and scarring also occurs during surgery to correct chronic sinusitis or removal of other regions of chronic inflammation (e.g., foreign bodies, infections (fungal, mycobacterium). Surgical procedures that may lead to surgical adhesions may include cardiac, spinal, neurologic, pleural, thoracic and gynaecologic surgeries. However, adhesions may also develop as a result of other processes, including, but not limited to, non-surgical mechanical injury, ischemia, hemorrhage, radiation treatment, infection-related inflammation, pelvic inflammatory disease and/or foreign body reaction. This abnormal scarring interferes with normal physiological functioning and, in come cases, can force and/or interfere with follow-up, corrective or other surgical operations. For example, these post-operative surgical adhesions occur in 60 to 90% of patients undergoing major gynaecologic surgery and represent one of the most common causes of intestinal obstruction in the industrialized world. These adhesions are a major cause of failed surgical therapy and are the leading cause of bowel obstruction and infertility. Other adhesion-treated complications include chronic pelvic pain, urethral obstruction and voiding dysfunction.

In one aspect, films and meshes may be used to prevent surgical adhesions in the epidural and dural tissue which is a factor contributing to failed back surgeries and complications associated with spinal injuries (e.g., compression and crush injuries). Scar formation within dura and around nerve roots has been implicated in rendering subsequent spine operations technically more difficult. To gain access to the spinal foramen during back surgeries, vertebral bone tissue is often disrupted. Back surgeries, such as laminectomies and diskectomies, often leave the spinal dura exposed and unprotected. As a result, scar tissue frequently forms between the dura and the surrounding tissue. This scar is formed from the damaged erector spinae muscles that overlay the laminectomy site. This results in adhesion development between the muscle tissue and the fragile dura, thereby, reducing mobility of the spine and nerve roots which leads to pain and slow post-operative recovery. To circumvent adhesion development, a scar-reducing barrier may be inserted between the dural sleeve and the paravertebral musculature post-laminotomy. This reduces cellular and vascular invasion into the epidural space from the overlying muscle and exposed cancellous bone and thus, reduces the complications associated with the canal housing the spinal chord and/or nerve roots.

The combination of compounds can be associated with an adhesion barrier that is a biodegradable or dissolvable film or mesh which is applied to the treatment site prior or post implantation of the prosthesis/implant. Exemplary materials for the manufacture of adhesion barriers are hyaluronic acid (crosslinked or non-crosslinked), cellulose derivatives (e.g., hydroxypropyl cellulose), PLGA, collagen and crosslinked poly(ethylene glycol). Alternatively, the device may be in the form of a tissue graft, which may be an autograft, allograft, biograft, biogenic graft or xenograft.

Additional examples of materials for use as adhesion barriers are described in “Composite Drug Delivery System,” filed Sep. 15, 2006 (U.S. Ser. No. 60/844,814) and “Composite Drug Delivery System,” filed Nov. 22, 2006 (U.S. Ser. No. ______ not yet assigned).

Adhesion barriers, which may be combined with a combination of compounds according to the present disclosure, include commercially available products, such as INTERCEED (Johnson & Johnson, Inc.), PRECLUDE (W. L. Gore), and POLYACTIVE (poly(ether ester) multiblock copolymers (Osteotech, Inc., Shrewsbury, N.J.), based on poly(ethylene glycol) and poly(butylene terephthalate), and SURGICAL absorbable hemostat gauze-like sheet from Johnson & Johnson. Another material is a prosthetic polypropylene mesh with a bioresorbable coating called SEPRAMESH Biosurgical Composite (Genzyme Corporation, Cambridge, Mass.). One side of the mesh is coated with a bioresorbable layer of sodium hyaluronate and carboxymethylcellulose, providing a temporary physical barrier that separates the underlying tissue and organ surfaces from the mesh. The other side of the mesh is uncoated, allowing for complete tissue ingrowth similar to bare polypropylene mesh. In one embodiment, the compounds may be applied only to the uncoated side of SEPRAMESH and not to the sodium hyaluronate/carboxymethylcellulose coated side. Other materials which may be used include: (a) BARD MARLEX mesh (C.R. Bard, Inc.), which is a very dense knitted fabric structure with low porosity; (b) monofilament polypropylene mesh such as PROLENE available from Ethicon, Inc. Somerville, N.J. (see, e.g., U.S. Pat. Nos. 5,634,931 and 5,824,082)); (c) SURGISIS GOLD and SURGISIS IHM soft tissue graft (both from Cook Surgical, Inc.) which are devices specifically configured for use to reinforce soft tissue in repair of inguinal hernias in open and laparoscopic procedures; (d) thin walled polypropylene surgical meshes such as are available from Atrium Medical Corporation (Hudson, N.H.) under the trade names PROLITE, PROLITE ULTRA, and LITEMESH; (e) COMPOSIX hernia mesh (C.R. Bard, Murray Hill, N.J.), which incorporates a mesh patch (the patch includes two layers of an inert synthetic mesh, generally made of polypropylene, and is described in U.S. Pat. No. 6,280,453) that includes a filament to stiffen and maintain the device in a flat configuration; (f) VISILEX mesh (from C.R. Bard, Inc.), which is a polypropylene mesh that is constructed with monofilament polypropylene; (g) other meshes available from C.R. Bard, Inc. which include PERFIX Plug, KUGEL Hernia Patch, 3D MAX mesh, LHI mesh, DULEX mesh, and the VENTRALEX Hernia Patch; and (h) other types of polypropylene monofilament hernia mesh and plug products include HERTRA mesh 1, 2, and 2A, HERMESH 3, 4 & 5 and HERNIAMESH plugs T1, T2, and T3 from Herniamesh USA, Inc. (Great Neck, N.Y.).

Other examples of commercially available meshes which may be combined with combinations of compounds include the following: TRELEX NATURAL Mesh (Boston Scientific Corporation), which is composed of a unique knitted polypropylene material; absorbable VICRYL (polyglactin 910) meshes (knitted and woven) and MERSILENE Polyester Fiber Mesh (Ethicon, Inc.); mesh material formed from silicone elastomer known as SILASTIC Rx Medical Grade Sheeting (Platinum Cured) (Dow Corning Corporation (Midland, Mich.); mesh made from absorbable polyglycolic acid under the trade name DEXON Mesh Products (United States Surgical/Syneture (Norwalk, Conn.); CELGARD microporous polypropylene fiber and membrane (Membrana Accurel Systems (Germany); oxidized, regenerated cellulose known as INTERCEED TC7 (Gynecare Worldwide, a division of Ethicon, Inc.); DURAGEN PLUS Adhesion Barrier Matrix, which can be used as a barrier against adhesions following spinal and cranial surgery and for restoration of the dura mater (Integra LifeSciences Corporation (Plainsboro, N.J.); and film for temporary wound support to control the formation of adhesions in specific spinal applications such as HYDROSORB Shield from MacroPore Biosurgery, Inc. (San Diego, Calif.).

As adhesion barriers are made in a variety of configurations and sizes, the exact dose of the administered compounds will vary with device size, surface area and design. Regardless of the method of application of the compounds to the device, the total amount (dose) of each compound in or on the device may be in the range of about 0.01 μg-10 μg, or 10 μg-10 mg, or 10 mg-250 mg, or 250 mg-1000 mg, or 1000 mg-2500 mg. The dose (amount) of each compound per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

In certain aspects, adhesion barriers are provided that are associated with a combination of paclitaxel and dipyridamole, where the total amount of each compound on, in or near the device may be in an amount ranging from less than 0.01 μg to about 2500 μg per mm² of device surface area. Generally, the compound(s) may be in an amount ranging from less than 0.01 μg; or from 0.01 μg to about 1.0 μg; or from 0.01 μg to about 10 μg; or from about 0.5 μg to about 5 μg; or from about 0.05 μg to 50 μg; or from 10 μg to about 250 μg; or from 250 μg to about 2500 μg (per mm² of device surface area).

In certain embodiments, paclitaxel is present in an amount ranging from about 0.01 to about 1.0 μg/mm² and dipyridamole is present in an amount ranging from about 0.05 to about 50 μg/mm².

In other embodiments, paclitaxel is present in an amount ranging from about 0.1 to about 0.6 μg/mm² and dipyridamole is present in an amount ranging from about 0.5 to about 5 μg/mm².

The total amount of each compound made available on, in or near the device may be in an amount ranging from about 0.01 μg (micrograms) to about 2500 mg (milligrams). Generally, the compounds may be in the amount ranging from 0.01 μg to about 10 μg; or from 10 μg to about 1 mg; or from 1 mg to about 10 mg; or from 10 mg to about 100 mg; or from 100 mg to about 500 mg; or from 500 mg to about 2500 mg.

In one embodiment, the weight ratio of dipyridamole to paclitaxel may exceed about 0.06 to about 1.0 to provide a superior biological effect. The weight ratio of dipyridamole to paclitaxel may be adjusted to exceed about 0.06; or about 0.08; or about 0.10; or about 0.20; or about 0.30 or about 0.40; or about 0.50; or about 0.60; or about 0.70; or about 0.80; or about 0.90; or about 1.0; or about 1.1; or about 1.2; or about 1.3; or about 1.4; or about 1.5; or about 1.6.

In one aspect, implantable sensors and drug-delivery pumps are provided which are associated with a combination of paclitaxel and dipyridamole.

“Implantable sensor” refers to a medical device that is implanted in the body to detect blood or tissue levels of a particular chemical (e.g., glucose, electrolytes, drugs, hormones) and/or changes in body chemistry, metabolites, function, pressure, flow, physical structure, electrical activity or other variable parameter. Implantable sensors may have one or more electrodes that extend into the external environment to sense a variety of physical and/or physiological properties, including, but not limited to, optical, mechanical, baro, chemical and electrochemical properties. Sensors may be used to detect information, for example, about temperature, strain, pressure, magnetic, acceleration, ionizing radiation, acoustic wave or chemical changes (e.g., blood constituents, such as glucose). For example for the detection of glucose levels, the sensor may utilize an enzyme-based electrochemical sensor, a glucose-responsive hydrogel combined with a pressure sensor, microwires with electrodes, radiofrequency microelectronics and a glucose affinity polymer combined with physical and biochemical sensor technology, and near or mid infrared light emission combined with optical spectroscopy detectors to name a few. Representative examples of implantable sensors include, blood/tissue glucose monitors, electrolyte sensors, blood constituent sensors, temperature sensors, pH sensors, optical sensors, amperometric sensors, pressure sensors, biosensors, sensing transponders, strain sensors, activity sensors and magnetoresistive sensors.

“Drug-delivery pump” refers to a medical device that includes a pump which is configured to deliver a biologically active agent (e.g., a drug) at a regulated dose. These devices are implanted within the body and may include an external transmitter for programming the controlled release of drug, or alternatively, may include an implantable sensor that provides the trigger for the drug delivery pump to release drug as physiologically required. Drug-delivery pumps may be used to deliver virtually any agent, but specific examples include insulin for the treatment of diabetes, medication for the relief of pain, chemotherapy for the treatment of cancer, anti-spastic agents for the treatment of movement and muscular disorders, or antibiotics for the treatment of infections. Representative examples of drug delivery pumps for use in the practice of this disclosure include, without limitation, constant flow drug delivery pumps, programmable drug delivery pumps, intrathecal pumps, implantable insulin delivery pumps, implantable osmotic pumps, ocular drug delivery pumps and implants, metering systems, peristaltic (roller) pumps, electronically driven pumps, elastomeric pumps, spring-contraction pumps, gas-driven pumps (e.g., induced by electrolytic cell or chemical reaction), hydraulic pumps, piston-dependent pumps and non-piston-dependent pumps, dispensing chambers, infusion pumps, passive pumps, infusate pumps and osmotically-driven fluid dispensers.

As implantable sensors and drug-delivery pumps are made in a variety of configurations and sizes, the exact dose of the administered compounds will vary with device size, surface area and design. Regardless of the method of application of the compounds to the intravascular device, the total amount (dose) of each compound in or on the device may be in the range of about 0.01 μg-10 μg, or 10 μg-10 mg, or 10 mg-250 mg, or 250 mg-1000 mg, or 1000 mg-2500 mg. The dose (amount) of each compound per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

In certain aspects, implantable sensors and drug-delivery pumps are provided that are associated with a combination of paclitaxel and dipyridamole, where the total amount of each compound on, in or near the device may be in an amount ranging from less than 0.01 μg to about 2500 μg per mm² of device surface area. Generally, the compound may be in an amount ranging from less than 0.01 μg; or from 0.01 μg to about 1.0 μg; or from 0.01 μg to about 10 μg; or from about 0.5 μg to about 5 μg; or from about 0.05 μg to 50 μg; or from 10 μg to about 250 μg; or from 250 μg to about 2500 μg (per mm² of device surface area).

In certain aspects, the weight ratio of dipyridamole to paclitaxel may be adjusted to provide a superior biological effect (e.g., to minimize formation of neointimal hyperplasia). In one embodiment, the weight ratio of dipyridamole to paclitaxel may exceed about 0.06 to about 1.0 to provide a superior biological effect. In other embodiments, the weight ratio of dipyridamole to paclitaxel may be adjusted to exceed about 0.06; or about 0.08; or about 0.10; or about 0.20; or about 0.30 or about 0.40; or about 0.50; or about 0.60; or about 0.70; or about 0.80; or about 0.90; or about 1.0; or about 1.1; or about 1.2; or about 1.3; or about 1.4; or about 1.5; or about 1.6.

Infiltration of Compositions Around Medical Devices and Implants

In another aspect, compositions are provided that include a combination of paclitaxel and dipyridamole (or analogues or derivatives thereof) may be infiltrated around implanted medical devices. Compositions may be infiltrated around implanted medical devices by applying the composition directly and/or indirectly into and/or onto (a) tissue adjacent to the medical device; (b) the vicinity of the medical device-tissue interface; (c) the region around the medical device; and (d) tissue surrounding the medical device. Methods for infiltrating the subject polymer compositions into tissue adjacent to a medical device include delivering the polymer composition: (a) to the medical device surface (e.g., as an injectable, paste, gel or mesh) during the implantation procedure; (b) to the surface of the tissue (e.g., as an injectable, paste, gel, in situ forming gel or mesh) immediately prior to, or during, implantation of the medical device; (c) to the surface of the medical device and/or the tissue surrounding the implanted medical device (e.g., as an injectable, paste, gel, in situ forming gel or mesh) immediately after the implantation of the medical device; (d) by topical application of the composition into the anatomical space where the medical device may be placed (particularly useful for this embodiment is the use of polymeric carriers which release the compound over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the medical device as a solution as an infusate or as a sustained release preparation; (f) by any combination of the aforementioned methods. In all cases it is understood that the subject polymer compositions may be infiltrated into tissue adjacent to all or a portion of the device.

Representative examples of polymer compositions that may be combined with the described compounds and infiltrated into or onto tissue adjacent to or in the vicinity of devices described herein include: (a) sprayable collagen-containing formulations such as COSTASIS (Angiotech Pharmaceuticals, Inc., Canada) and crosslinked poly(ethylene glycol)-methylated collagen compositions (described, e.g., in U.S. Pat. Nos. 5,874,500 and 5,565,519); (b) sprayable PEG-containing formulations such as COSEAL (Angiotech Pharmaceuticals, Inc.), FOCALSEAL (Genzyme Corporation, Cambridge, Mass.), SPRAYGEL or DURASEAL (both from Confluent Surgical, Inc., Boston, Mass.); (c) fibrinogen-containing formulations such as FLOSEAL or TISSEAL (both from Baxter Healthcare Corporation, Fremont, Calif.); (d) hyaluronic acid-containing formulations such as RESTYLANE or PERLANE (both from Q-Med AB, Sweden), HYLAFORM (Inamed Corporation, Santa Barbara, Calif.), SYNVISC (Biomatrix, Inc., Ridgefield, N.J.), SEPRAFILM or SEPRACOAT (both from Genzyme Corporation); (e) polymeric gels for surgical implantation such as REPEL (Life Medical Sciences, Inc., Princeton, N.J.) or FLOWGEL (Baxter Healthcare Corporation); (f) surgical adhesives containing cyanoacrylates such as DERMABOND (Johnson & Johnson, Inc.), INDERMIL (U.S. Surgical Company, Norwalk, Conn.), GLUSTITCH (Blacklock Medical Products Inc., Canada), TISSUEMEND (Veterinary Products Laboratories, Phoenix, Ariz.), VETBOND (3M Company, St. Paul, Minn.), HISTOACRYL BLUE (Davis & Geck, St. Louis, Mo.) and ORABASE SOOTHE-N-SEAL LIQUID PROTECTANT (Colgate-Palmolive Company, New York, N.Y.); (h) other biocompatible tissue fillers, such as those made by BioCure, Inc. (Norcross, Ga.), 3M Company (St. Paul, Minn.) and Neomend, Inc. (Sunnyvale, Calif.); (i) polysaccharide gels such as the ADCON series of gels (available from Gliatech, Inc., Cleveland, Ohio); and/or (k) films, sponges or meshes such as INTERCEED (Gynecare Worldwide, a division of Ethicon, Inc., Somerville, N.J.), VICRYL mesh (Ethicon, Inc.), and GELFOAM (Pfizer, Inc., New York, N.Y.).

In one aspect, the subject polymer compositions may be infiltrated into or onto tissue adjacent to an intravascular device (e.g., cardiovascular stent, coronary stent, peripheral stent, intravascular balloon or catheter, guidewire, and the like).

In one aspect, the subject polymer compositions may be infiltrated into or onto tissue adjacent to a non-vascular stent (e.g., tracheal stent, bronchial stent, GI stent, and the like)

In one aspect, the subject polymer compositions may be infiltrated into or onto tissue adjacent to an anastomotic connector device.

In one aspect, the subject polymer compositions may be infiltrated into or onto tissue adjacent to vascular graft.

In one aspect, the subject polymer compositions may be infiltrated into or onto tissue adjacent to perivascular device.

In one aspect, the subject polymer compositions may be infiltrated into or onto tissue adjacent to a breast implant.

In one aspect, the subject polymer compositions may be infiltrated into or onto tissue adjacent to a facial or aesthetic implant.

In one aspect, the subject polymer compositions may be infiltrated into or onto tissue adjacent to tissue filler.

In one aspect, the subject polymer compositions may be infiltrated into or onto tissue adjacent to an electrical lead.

In one aspect, the subject polymer compositions may be infiltrated into or onto tissue adjacent to an implantable pump or sensor.

In one aspect, the subject polymer compositions may be infiltrated into or onto tissue adjacent to a venous filter device (such as a vena cava filter).

In certain aspects, compositions are provided that are associated with a combination of paclitaxel and dipyridamole, where the total amount of each compound on, in or near the device may be in an amount ranging from less than 0.01 μg to about 2500 μg per mm² of device surface area. Generally, the compound(s) may be present in an amount ranging from less than 0.01 μg; or from 0.01 μg to about 1.0 μg; or from 0.01 μg to about 10 μg; or from about 0.5 μg to about 5 μg; or from about 0.05 μg to 50 μg; or from 10 μg to about 250 μg; or from 250 μg to about 2500 μg (per mm² of device surface area).

In certain embodiments, paclitaxel is present in an amount ranging from about 0.01 to about 1.0 μg/mm² and dipyridamole is present in an amount ranging from about 0.05 to about 50 μg/mm².

In other embodiments, paclitaxel is present in an amount ranging from about 0.1 to about 0.6 μg/mm² and dipyridamole is present in an amount ranging from about 0.5 to about 5 μg/mm².

The total amount of each compound made available on, in or near the device may be in an amount ranging from about 0.01 μg (micrograms) to about 2500 mg (milligrams). Generally, the compounds may be in the amount ranging from 0.01 μg to about 10 μg; or from 10 μg to about 1 mg; or from 1 mg to about 10 mg; or from 10 mg to about 100 mg; or from 100 mg to about 500 mg; or from 500 mg to about 2500 mg.

In one embodiment, the weight ratio of dipyridamole to paclitaxel may exceed about 0.06 to about 1.0 to provide a superior biological effect. The weight ratio of dipyridamole to paclitaxel may be adjusted to exceed about 0.06; or about 0.08; or about 0.10; or about 0.20; or about 0.30 or about 0.40; or about 0.50; or about 0.60; or about 0.70; or about 0.80; or about 0.90; or about 1.0; or about 1.1; or about 1.2; or about 1.3; or about 1.4; or about 1.5; or about 1.6.

The following examples are offered by way of illustration, and not by way of limitation. The contents of all figures and all references, patents and published patent applications cited throughout this application are expressly incorporated herein by reference.

### EXAMPLES

### Example 1

### Coating Solutions

Stainless steel stents (Pulse Systems, Inc., Concord, Calif.) were plasma treated and then spray coated with the following primer solution and dried in an oven for 30 minutes at 125-130° C. The coating and drying procedure was repeated a second time.

Coating Solution A

| Component | Amount (grams) |
|---|---|
| Ethylene acrylic acid copolymer | 1.68 |
| Tetrahydrofuran (THF) | 15.54 |
| Dimethyl acetamide (DMAC) | 19.87 |
| Anisole | 21.27 |
| Xylenes | 41.34 |
| Epoxy resin | 0.33 |

The devices were then spray coated with the following solution and dried in an oven at 125-130° C. for 30 minutes. The coating and drying procedure was repeated a second time to form an intermediate (tie layer).

Coating Solution B

| Component | Amount (grams) |
|---|---|
| Aromatic polycarbonate-based polyurethane | 11.03 |
| solution (22-25% by weight in DMAC) | |
| Dimethyl acetamide (DMAC) | 0.27 |
| Anisole | 20.22 |
| Methyl isobutyl ketone (MIBK) | 68.48 |

Paclitaxel and dipyridamole were added to polymer stock solutions in various amounts to produce the following coating solutions.

Coating Solution C

| Component | Amount (grams) |
|---|---|
| Aromatic polycarbonate-based polyurethane | 9.01 |
| solution (22-25% by weight in DMAC) | |
| Nitrocellulose | 1.36 |
| Dipyridamole | 0.28 |
| Paclitaxel | 1.40 |
| Anisole | 27.19 |
| Methylethylketone (MEK) | 29.50 |
| DMAC | 11.61 |
| n-Butanol | 19.67 |

Coating Solution D

| Component | Amount (grams) |
|---|---|
| Aromatic polycarbonate-based polyurethane | 7.85 |
| solution (22-25% by weight in DMAC) | |
| Nitrocellulose | 1.63 |
| Dipyridamole | 1.00 |
| Paclitaxel | 0.20 |
| Anisole | 27.32 |
| Methylethylketone (MEK) | 29.64 |
| DMAC | 12.60 |
| n-Butanol | 19.76 |

Coating Solution E

| Component | Amount (grams) |
|---|---|
| Aromatic polycarbonate-based polyurethane | 7.89 |
| solution (22-25% by weight in DMAC) | |
| Nitrocellulose | 1.64 |
| Dipyridamole | 0.36 |
| Paclitaxel | 0.34 |
| Anisole | 27.46 |
| Methylethylketone (MEK) | 29.79 |
| DMAC | 12.66 |
| n-Butanol | 19.86 |

Devices coated with Coating Solution A and B were spray coated with Coating Solution C, D, or E and dried in an oven for 30 minutes at 75±5° C. The process was repeated to obtain the desired compound loading. After a sufficient number of layers had been applied, the devices were dried under vacuum for 1 hour at 75±10° C. The process generated thin, flexible coatings that adhered well to the stents under wet and dry conditions.

### Example 2

### More Coating Solutions

Stainless steel stents (Pulse Systems, Inc., Concord, Calif.) were plasma treated and then spray coated with the following primer solution and dried in an oven for 30 minutes at 125-130° C. The coating and drying procedure was repeated a second time.

Coating Solution F

| Component | Amount (grams) |
|---|---|
| Styrene-isobutylene-styrene copolymer | 1.00 |
| Ethylene acrylic acid copolymer | 1.66 |
| Tetrahydrofuran (THF) | 15.38 |
| Dimethyl acetamide (DMAC) | 19.67 |
| Anisole | 21.06 |
| Xylenes | 40.93 |
| Epoxy resin | 0.33 |

The devices were then spray coated with the following solution and dried in an oven at 125-130° C. for 30 minutes. The solution was re-applied and dried for 60 minutes to form an intermediate (tie layer).

Coating Solution G

| Component | Amount (grams) |
|---|---|
| Styrene-isobutylene-styrene copolymer | 3.50 |
| Toluene | 91.55 |
| THF | 4.95 |

The devices were then spray coated with the one of the following polymer solutions and dried in an oven for 30 minutes at 75±5° C. The process was repeated to obtain the desired compound loading. After a sufficient number of layers had been applied, the devices were dried under vacuum for 1 hour at 75±10° C.

Coating Solution H

| Component | Amount (grams) |
|---|---|
| Styrene-isobutylene-styrene copolymer | 3.50 |
| Paclitaxel | 0.34 |
| Toluene | 89.83 |
| DMAC | 6.33 |

Coating Solution I

| Component | Amount (grams) |
|---|---|
| Styrene-isobutylene-styrene copolymer | 3.44 |
| Dipyridamole | 1.39 |
| Paclitaxel | 0.28 |
| Toluene | 88.21 |
| DMAC | 6.69 |

Coating Solution J

| Component | Amount (grams) |
|---|---|
| Styrene-isobutylene-styrene copolymer | 3.50 |
| Dipyridamole | 0.18 |
| Paclitaxel | 0.16 |
| Toluene | 89.83 |
| DMAC | 6.33 |

Coating Solution K (Control)

| Component | Amount (grams) |
|---|---|
| Styrene-isobutylene-styrene copolymer | 3.50 |
| Toluene | 90.14 |
| DMAC | 6.36 |

### Example 3

### Procedure for Producing SIS Films

Paclitaxel, dipyridamole, or a combination of paclitaxel and dipyridamole were incorporated into styrene-isoprene-styrene (SIS) polymeric films. Two grams (2 g) of styrene-isoprene-styrene polymer (Mₙ=150K dalton/mole by GPC relatively to PS standard, Sigma-Aldrich) was dissolved in 10 mL tetrahydrofuran to achieve a 20% w/v solution and loaded with various amounts of paclitaxel and/or dipyridamole. The drug loaded solutions were cast into a film (50×130 mm²) and the film was dried under nitrogen for 1 hour at room temperature and then at 40° C. in a forced-air oven for 2 hours. The film was further vacuum-dried for 16 hours at room temperature. The final film was cut into 8 mm×8 mm using a die cutter. The films had a thickness of about 55-60 μm. Films having the following amounts of paclitaxel and dipyridamole were prepared: paclitaxel (3, 10, 30 μg); dipyridamole (50 μg); dipyridamole/paclitaxel (50/3 μg; 50/10 μg; 100/3 μg; 150/3 μg; and 150/10 μg).

### Example 4

### Inhibition of Angiogenesis by Paclitaxel and Dipyridamole

Paclitaxel, dipyridamole, and a combination of paclitaxel and dipyridimole were tested in a chick chorioallantoic membrane (CAM) assay (A. Cevik Tufan and N. Lalae Satirglu-Tufanm Current Cancer Drug Targets, 2005, 5: 249-266) to measure inhibition of angiogenesis by the compounds.

Fertilized, domestic chick embryos were incubated for 4 days prior to shell-less culturing. In this procedure, the egg contents were emptied by cracking the shell, and allowing the contents of the egg to gently slide out. The egg contents were emptied into sterilized petri dishes and then covered with petri dish covers. These were then placed into an incubator at 37 degrees and 90% relative humidity for 4 days.

Paclitaxel (Hauser Lot 1492-16199A) was fixed at concentrations of 0.3 μg and 1 μg per 10 ul aliquot of 0.5% aqueous methylcellulose (disc). Dipyridamole (Aldrich 285676, Lot 064K157) was added to each fixed dose of paclitaxel at specific molar ratios of 1:3, 1:10, 1:1, 3:1, 10:1. Neat solvent (DMSO), paclitaxel at 0.3 μg and 1 μg per disc and the dipyridamole at 5.92 μg per disc were used as the individual controls. Ten microliter aliquots of this solution were dried on parafilm for 3 hours forming disks 2 mm in diameter. The dried disks containing the combination ratios and controls were then carefully placed at the growing edge of each CAM at day 7 of incubation. After a 2 day exposure (day 8 of incubation) the vasculature was examined with the aid of a stereomicroscope. Liposyn II, a white opaque solution, was injected into the CAM to increase the visibility of the vascular details.

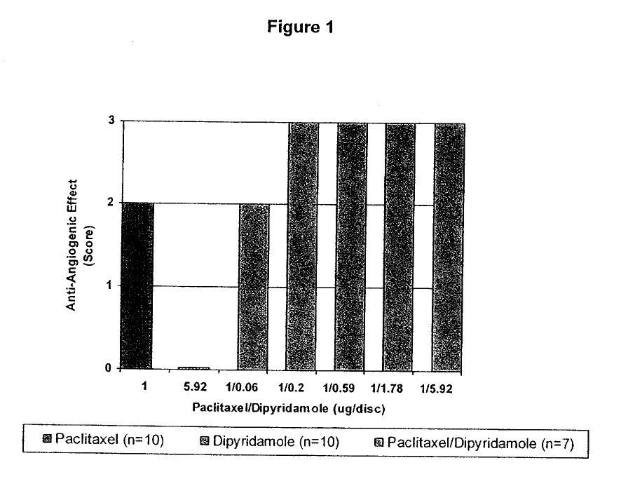

This imaging setup was used at a magnification of 160× which permitted the direct visualization of blood cells within the capillaries; thereby blood flow in areas of interest may be easily assessed and recorded. For this study, the inhibition of angiogenesis was defined as an area of the CAM (measuring 2-6 mm in diameter) lacking a capillary network and vascular blood flow. Throughout the experiments, avascular zones were assessed on a 4 point avascular gradient (Table 1). This scale represents the degree of overall inhibition with maximal inhibition represented as a 3 on the avascular gradient scale. The results of the study are shown in Table 2 and FIG. 1.

TABLE 1
Avascular Gradient Scale

0 -- normal vascularity
1 -- lacking some microvascular movement
2* -- small avascular zone approximately 2 mm in diameter
3* -- avascularity extending beyond the disk (6 mm in diameter)
*indicates a positive antiangiogenesis response

TABLE 2
Summary of CAM Assay Results

| | | Compound Ratios | | |
|---|---|---|---|---|
| | Number of | Paclitaxel | Dipyridamole | |
| Samples | Eggs/Group | (μg/10 μL) | (μg/10 μL) | Score |
| 10% DMSO (control) | 10 | 0 | 0 | 0 |
| Paclitaxel (control) | 10 | 1 | 0 | 2 |
| Dipyridamole (control) | 10 | 0 | 0.02 | 0 |
| Dipyridamole (control) | 10 | 0 | 0.06 | 0 |
| Dipyridamole (control) | 10 | 0 | 5.92 | 0 |
| Ratio 1 (10:1) | 7 | 1 | 0.06 | 2 |
| Ratio 2 (3:1) | 7 | 1 | 0.20 | 2 |
| Ratio 3 (1:1) | 7 | 1 | 0.59 | 3 |
| Ratio 4 (1:3) | 7 | 1 | 1.78 | 3 |
| Ratio 5 (1:10) | 7 | 1 | 5.92 | 3 |

The studies demonstrated that paclitaxel at a dose of 1 μg/10 μl disc reproducibly yielded a score of 2 on the CAM assay. Dipyridamole alone at doses of 0.02, 0.06, and 5.92 μg/10 μl disc produced scores of 0. A combination of paclitaxel and dipyridamole at ratios of 1:1, 1:3, and 1:10 (1 μg/10 μl disc paclitaxel and 0.59, 1.78, or 5.92 μg/10 μl disc dipyridamole) potentiated anti-angiogenesis with scores of 3.

### Example 5

### Evaluation of Paclitaxel and Dipyridamole on Intimal Hyperplasia Development in a Rat Balloon Injury Carotid Artery Model

A rat balloon injury carotid artery model was used to evaluate the efficacy following placement of styrene-isoprene-styrene (SIS) films loaded with paclitaxel, dipyridamole, and a combination of paclitaxel and dipyridamole (prepared as in Example 2).

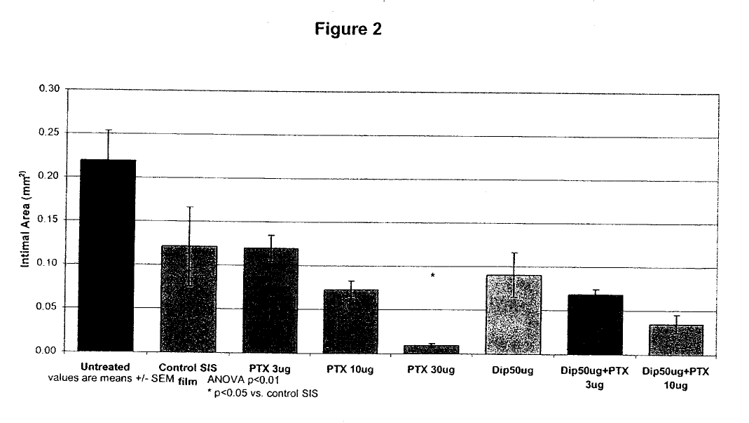

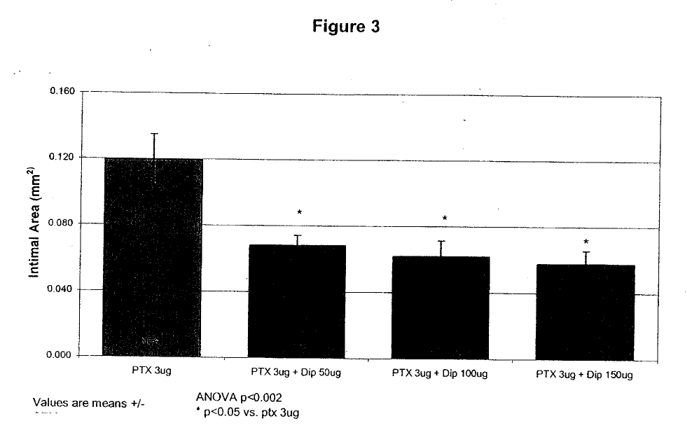

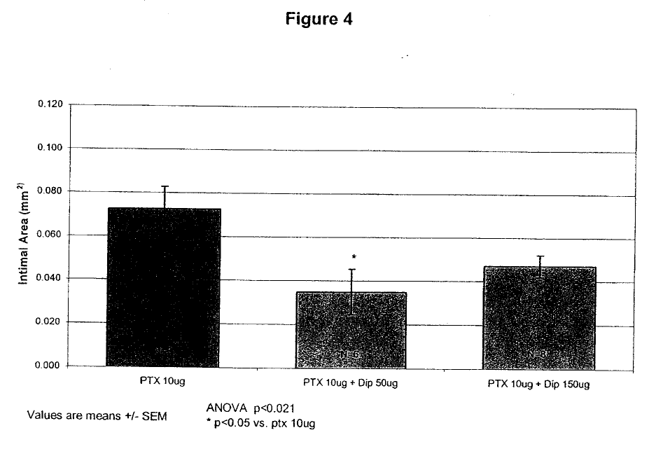

A 2-French Fogarty arterial embolectomy catheter was introduced through the incision in the left external carotid artery of rats and advanced proximally into the left common carotid artery. The balloon was inflated with 0.02 mL saline and was retracted distally along the entire length of the left common carotid artery. The balloon was deflated and the procedure repeated a total of 3 times. Afterward the catheter was removed and left external carotid artery was tied off. A drug-loaded SIS film or a control film was wrapped around the carotid artery of each balloon-injured animal and the animal was allowed to recover. At 14 days, animals were sacrificed and morphometric analysis was used to measure intimal hyperplasia. The results are summarized in FIGS. 2, 3 and 4.

### Example 6

### Evaluation of Stents in Porcine Coronary Artery Model

This protocol outlines the procedure for a 28 day study to assess the feasibility of implanting stents coated with styrene-isobutylene-styrene (SIBS) block copolymer loaded with a combination of paclitaxel and dipyridamole in porcine coronary arteries.

The drug eluting stents used in the study are generic electropolished stainless steel stents coated with paclitaxel and/or dipyridamole loaded in SIBS polymer. The stents are crimped on a rapid-exchange balloon catheters.

Four groups of stents are to be tested. A bare metal stent group (Group 1; n=3 stents) is used to assess the safety of the stent platform in this model. A polymer only group (Group 2; n=3 stents) is used to assess the safety of the SIBS polymer coating in this model. Group 3 stents (n=3) are loaded with paclitaxel (1 μg/mm²; 72 μg total dose) in SIBS polymer. Group 4 stents (n=3 stents) are loaded with a combination of paclitaxel (0.6 μg/mm²; 43 μg total dose) and dipyridamole (2.1 μg/mm²; 150 μg total dose) in SIBS polymer.

After induction of anesthesia, the left femoral artery of the subject animal is accessed with an incision made in the inguinal region. Under fluoroscopic guidance, a guide catheter is inserted through the femoral artery and advanced to the coronary arteries. Angiographic images of the coronaries are obtained to identify the proper location for the deployment site. A guidewire is inserted into the chosen artery. Quantitative Coronary Angiography (QCA) is performed at this time to document the reference diameter for stent placement.

A stent is introduced into the chosen artery by advancing the stented balloon catheter through the guide catheter and over the guidewire to the deployment site. The balloon is then inflated at a steady rate to deploy the stent. An angiogram of the balloon at full inflation is recorded. Vacuum is applied to the inflation device in order to deflate the balloon. The delivery system is slowly removed. A last angiogram is recorded to document device patency. Implantation is repeated in the other vessels but may vary depending on the vessel anatomy and suitability for stenting. Following successful deployment of the stents and completion of angiography, all catheters are removed from the animals and the femoral artery is ligated. The incision is closed in layers with appropriate suture materials and the animal is allowed to recover from anesthesia and is kept for 28 days.

Twenty eight (28) days after implantation, the animals are tranquilized, weighed and anesthetized. An angiogram of the stented vessels is performed. The animals are euthanized and their hearts are perfused with 10% buffered formalin and immersed in 10% buffered formalin until processed for histology.

The fluoroscopic images from stent implantation and explantation are recorded. QCA measurements are obtained using Medis QCA-CMS 6.0 system and stenosis within the stent is quantified.

Stented arteries are harvested and processed for histology. Stented arteries are embedded in methyl methacrylate and cut in three blocks covering the proximal, mid and distal segments. Thin sections from each artery block are stained with hematoxylin and eosin (H&E) and an elastin stain. Elastin stain sections of arteries are evaluated to determine histomorphometric parameters. H&E sections are assessed to determine other histopathological parameters.

Histomorphometry is performed by quantitative morphometric computer-assisted methods using an image analysis software. The histology sections are digitized, and the amount of intimal growth and luminal narrowing is quantified.

Semi-quantitative parameters such as vessel injury, inflammation, fibrin deposition, endothelial loss are employed to assess the biological response of vascular tissue to the stents by light microscopy examination of stained sections.

1. A device comprising a medical device, paclitaxel and dipyridamole, wherein paclitaxel is present in an amount ranging from about 0.01 to about 1.0 μg/mm² and dipyridamole is present in an amount ranging from about 0.05 to about 50 μg/mm² of medical device surface area.

2. The device of claim 1 wherein paclitaxel is present in an amount ranging from about 0.1 to about 0.6 μg/mm² and dipyridamole is present in an amount ranging from about 0.5 to about 5 μg/mm² of medical device surface area.

3. A device comprising a medical device, paclitaxel and dipyridamole, wherein paclitaxel is present in an amount ranging from about 0.01 to about 1.0 μg/mm² and dipyridamole is present in an amount ranging from about 0.01 to about 1.0 μg/mm² of medical device surface area.

4. The device of claim 1 further comprising a polymer.

5. The device of claim 4 wherein the polymer is a non-biodegradable polymer.

6. The device of claim 4 wherein the polymer is a biodegradable polymer.

7. The device of claim 1 wherein the medical device is an intravascular device selected from a catheter, a balloon, and a vena cava filter.

8. The device of claim 1 wherein the medical device is selected from drug delivery pumps, sensors, non-vascular stents, vascular grafts, perivascular devices, implants for hemodialysis access, implants for providing an anastomotic connection, electrical devices, intraocular implants, and soft tissue implants and tissue fillers.

9. The device of claim 1 wherein the medical device is a coronary stent or a peripheral vascular stent.

10. The device of claim 1 wherein the paclitaxel has a biological effect, and the effect is greater in the presence of dipyridamole than in the absence of dipyridamole, and the biological effect is to minimize formation of neointimal hyperplasia.

11. A composition comprising paclitaxel and dipyridamole, wherein the weight ratio of dipyridamole to paclitaxel exceeds 0.06 to 1.0.

12. The composition of claim 11 wherein the paclitaxel has a biological effect, and the biological effect is greater in the presence of dipyridamole than in the absence of dipyridamole.

13. The composition of claim 11 comprising a combination of paclitaxel and dipyridamole, wherein the biological effect of the combination is greater than the sum of the effects of dipyridamole or paclitaxel acting alone.

14. The composition of claim 11 wherein the composition further comprises a polymer.

15. The composition of claim 14 wherein the polymer is a non-biodegradable polymer.

16. The composition of claim 14 wherein the polymer is a biodegradable polymer.

17. The device of claim 3 further comprising a polymer.

18. The device of claim 3 wherein the medical device is an intravascular device selected from a catheter, a balloon, and a vena cava filter.

19. The device of claim 3 wherein the medical device is selected from drug delivery pumps, sensors, non-vascular stents, vascular grafts, perivascular devices, implants for hemodialysis access, implants for providing an anastomotic connection, electrical devices, intraocular implants, and soft tissue implants and tissue fillers.

20. The device of claim 3 wherein the medical device is a coronary stent or a peripheral vascular stent.

21. The device of claim 3 wherein the paclitaxel has a biological effect, and the effect is greater in the presence of dipyridamole than in the absence of dipyridamole, and the biological effect is to minimize formation of neointimal hyperplasia.
